SEQUENCE LISTING (94 sequences)

SEQ ID NO: 1 - DNA, 1779 bp, synthetic construct
  source 1..1779: mol_type = other DNA
atgtggaccctcgtttcttgggtcgccctgacagccgggctggttgccggcactcggtgccctgacggacagttttgccccgtggcttgttgtcttgatccagggggggccagttactcttgttgtaggccattgcttgacaaatggccaaccactctttccaggcacctgggaggaccttgtcaggtggacgctcactgcagcgctggccacagctgtattttcacagtgtctgggacatcatcctgttgtcctttccccgaagcggtggcttgtggcgatggccatcattgctgtcctcggggatttcattgtagcgcagacggacgcagctgctttcaacggtctggaaacaattccgtcggggccatacaatgcccagattctcagtttgaatgccccgatttctctacctgttgcgtgatggtggacggctcctggggctgctgtcctatgccccaggcctcctgctgcgaggacagggtgcattgttgtcctcatggggccttctgtgacctcgttcacacaaggtgtattacaccaacaggcactcaccctctggccaagaaacttcctgcccagagaaccaatagggcagttgctctgagctcttcagtgatgtgccctgatgcccgctctcgctgtcctgacggcagcacatgctgtgaactcccttcaggcaagtatggatgttgtcctatgcccaatgcaacttgctgttccgaccacctccattgctgtcctcaagatacagtctgtgacctcatacagtcaaagtgcctttccaaagaaaacgccacaaccgatcttttgacaaagcttcccgcccacacagttggcgatgtgaagtgtgatatggaggtgtcctgccctgatggatatacttgctgccgcctgcaaagtggagcatggggctgctgtccattcacccaagccgtgtgctgcgaggatcatatccactgttgccctgctgggtttacgtgcgatactcaaaaaggcacctgcgaacagggaccacaccaagttccctggatggagaaagctccagcacatctttctttgccggatccacaggccctcaagcgggatgtcccatgcgataatgtgtccagctgcccgagttctgacacctgctgtcagttgacttctggagagtggggatgttgcccaattccagaggctgtttgctgttcagatcatcaacactgttgcccacagggttatacctgtgtcgctgaaggccaatgtcaacggggctctgagatcgttgcgggcctcgagaagatgcccgcccggcgcgcctctttgagccacccaagagacatcggatgcgaccagcacacctcatgtcctgtcggacagacttgttgtccttccctgggtggatcttgggcgtgttgtcaactcccccatgcagtatgctgcgaagataggcaacattgctgcccagctgggtatacctgtaatgtcaaggctagatcatgcgaaaaagaggttgtttctgctcagccagccacatttctcgcacggagccctcatgtgggggttaaagacgtcgagtgtggcgaagggcatttctgtcacgacaatcaaacatgttgtcgggataaccggcagggatgggcatgttgtccctatcggcaaggcgtgtgctgtgccgacagacggcattgctgtccagctgggttcagatgtgcagcaagagggactaaatgtcttcggagggaggccccccggtgggatgcaccccttcgggaccccgccctgagacagctgctg

SEQ ID NO: 2 - DNA, 1779 bp, synthetic construct
  source 1..1779: mol_type = other DNA
atgtggacactcgttagttgggttgccttgaccgctggattggtcgccggaacacgctgtcctgatggacagttttgtccagtcgcatgctgtttggatcctgggggcgctagttacagctgctgcaggccactgctcgataaatggccgactacattgtcacgccatttgggagggccttgccaagtcgatgctcactgttccgccggtcattcttgcattttcacagtttccggcactagctcatgctgcccgtttccggaagccgttgcctgtggcgatgggcatcattgctgtcctcggggctttcactgcagcgccgacggaaggtcctgcttccagaggtcagggaacaatagcgttggagcgattcaatgccctgactctcaatttgagtgtccagattttagcacctgctgtgtcatggtcgatggatcttggggctgttgccccatgcctcaagcgtcatgttgcgaggatcgcgtccactgttgtccacacggagccttttgtgacctggtacatactcggtgcatcacaccaaccgggacgcaccctttggccaaaaagcttcccgctcagagaacaaatcgggccgtcgcactcagtagttccgtaatgtgtcccgacgctcgctctcggtgcccagacggctctacatgctgcgagctgccgtcagggaagtatggctgctgtcctatgcccaacgctacttgttgttcagaccacctccattgctgtcctcaggataccgtttgtgatctcattcaaagtaagtgtctgtccaaagaaaatgctactaccgacttgctgactaaacttccagctcatacagtgggggatgtgaaatgcgacatggaagtctcttgtccagacgggtatacatgctgtaggctgcagtcaggggcctggggatgctgtccttttactcaagctgtttgttgtgaagaccatatacactgctgccccgcaggctttacctgtgatacccagaaaggaacatgtgagcagggtccacatcaagtgccttggatggagaaagcccccgctcacctttctcttcctgacccgcaggcgttgaagagggacgtgccttgtgacaatgtttcatcatgtccaagctctgacacctgttgtcaactgacatctggcgagtggggatgttgcccaatcccagaagccgtctgttgttctgatcaccaacactgttgcccacagggctatacctgcgtggccgaaggtcaatgccaaagagggagcgagattgttgcgggactggaaaaaatgcccgctcggagggccagcctgtcccacccacgagacataggatgtgaccagcatacctcttgtccggttggtcagacgtgctgcccaagcttggggggatcttgggcatgttgtcaactgccgcacgcagtttgttgcgaagacagacaacactgctgtcccgcgggctacacttgtaacgttaaggcaagaagctgcgaaaaagaggtggtttctgcccagccagcaaccttcctcgctagatcccctcacgttggcgtgaaggacgtggagtgcggcgagggacatttctgtcacgataaccaaacctgctgtcgcgacaaccgccagggttgggcctgctgcccttatcgccagggagtgtgctgtgccgatcggcgccattgttgtcctgcagggtttagatgcgctgctaggggtactaaatgtctgagaagggaagctccgagatgggatgcccctttgagagatccagctctgagacaattgctc

SEQ ID NO: 3 - DNA, 1779 bp, synthetic construct
  source 1..1779: mol_type = other DNA
atgtggacacttgtttcatgggttgcactcaccgctggactggtcgctggaaccagatgtccagacggacagttttgcccagtggcatgctgtctcgatcctggcggagctagctactcttgctgcagaccactgttggataagtggccaacaacactgagcagacatctgggcggcccatgccaagtggatgctcattgtagcgccggccactcctgcattttcactgtgtccggtacctcctcatgctgcccattccctgaagccgtcgcatgcggagatgggcatcactgttgccccagaggttttcactgcagcgctgatgggcggtcttgcttccaaaggtccggaaacaatagcgttggggcgattcagtgccctgactcccaatttgaatgccctgacttttcaacctgttgtgtaatggtcgatggcagctggggctgctgccccatgccacaggcttcttgttgcgaagaccgcgtgcattgttgccctcatggagcgttttgtgacctcgtgcacacgaggtgcattactcccacaggcactcatccactggctaagaaattgcctgcccagagaacaaacagggcagtagccttgagctcttcagtaatgtgtccggacgccagatcaaggtgccccgatggatctacttgctgcgagctcccctccggcaagtacggatgttgtccaatgccaaatgctacttgctgtagtgaccacctgcactgttgtccacaggatacggtctgtgacttgatacagtctaagtgcctgtctaaggaaaatgcaacaaccgatttgcttacaaaactgccagcccataccgtaggagatgtcaagtgcgacatggaagttagttgtcccgacggctatacgtgttgtcgactgcaatccggagcctggggttgttgcccgtttactcaggctgtatgttgcgaggaccacatccactgttgtcccgcggggttcacctgtgacacccagaaaggtacctgcgagcaggggcctcatcaggtaccgtggatggaaaaagctcctgcacacttgtcactgccggatcctcaggcgctcaagcgggatgtcccttgcgacaatgtaagctcctgtccgtctagtgatacgtgttgccaacttacctccggggaatggggctgttgccctataccagaagctgtctgttgctctgatcatcaacattgctgcccccagggttacacatgtgtagcagagggtcagtgtcaaagagggtcagaaattgtagctggcttggaaaaaatgcccgcaaggcgcgcatccctgtctcaccctcgggatataggatgcgatcagcacacctcttgtccagtgggtcagacttgttgtcccagcttggggggcagctgggcttgttgtcaattgcctcacgcagtttgctgtgaagaccgccaacattgctgtcctgctggatacacatgcaatgtcaaggctaggagctgtgaaaaagaggtggtgtctgcacagcccgcaacctttcttgcacgctctcctcatgtcggagtgaaagatgttgaatgcggagaaggacacttttgccatgacaatcagacttgttgcagggacaacagacaaggatgggcttgttgcccttatcgccaaggagtatgctgtgcggatagaagacattgttgcccggccgggtttaggtgtgctgctagaggcaccaaatgtctgaggcgagaagcacctagatgggacgctccactgcgggaccctgccttgagacaactcctg

SEQ ID NO: 4 - DNA, 1779 bp, synthetic construct
  source 1..1779: mol_type = other DNA
atgtggaccctcgtgtcctgggtggcactgacagccgggttggtggctggaactagatgcccagacggacaattctgtcccgtggcttgttgcttggaccctggaggcgcatcctatagctgttgcaggcccttgctggacaaatggccaaccacactgtctcggcatttgggcggcccatgccaggtggatgctcattgcagcgccggccacagctgcatcttcaccgtctccggaacaagcagctgttgtccatttccagaagcggtcgcttgtggcgatggacatcactgttgtcctcgcggatttcactgctctgccgatggaaggtcctgctttcaacggagcggaaataattctgtcggggccattcaatgtcccgactcccagttcgagtgccctgattttagcacatgttgtgtcatggtcgatggcagctggggatgctgcccaatgccacaggcatcctgctgcgaagatagggtacattgttgccctcatggagccttttgtgatctcgttcatacaaggtgtattactccaactggcacacatcccctcgccaagaaattgcctgcacagagaaccaaccgggctgtggccctctcttcctctgttatgtgtccagacgcacggtctagatgtcccgatggatctacatgttgcgaactgcctagcggaaagtatgggtgctgtcccatgcctaacgctacgtgctgctctgatcatttgcactgctgtccacaggatacagtgtgtgatctgattcaaagcaaatgcctgtccaaagaaaatgcaacaaccgatctcttgactaaactccctgcccacaccgtgggcgacgtaaagtgtgacatggaggtctcttgcccagacggctatacatgctgtcggctgcagtctggagcctggggctgttgccccttcactcaggccgtctgttgcgaggatcatatccattgttgtccagcaggatttacttgcgacacccagaaaggcacctgcgagcaaggcccacaccaagtgccttggatggagaaggccccagctcacctcagcctgcctgaccctcaggcactcaagagggatgtgccatgtgacaatgtttcttcctgcccttccagcgatacatgttgtcaattgacatctggcgaatggggatgttgtcccatcccagaggctgtgtgctgctccgatcaccagcactgttgtccacaggggtatacatgtgttgctgaaggccaatgtcaacggggctccgaaatcgtggcaggactcgaaaaaatgcctgcaaggagagctagcctctctcatccacgcgatatcggatgcgaccaacatacatcctgcccagtcggccaaacttgctgtccctcacttggaggttcctgggcttgctgccagctgccccatgccgtgtgctgcgaagatagacagcactgttgccctgccgggtacacctgcaatgtcaaggccagatcatgcgaaaaagaggtggtcagcgcacagccagctacattcttggccagatccccacatgtgggcgtgaaagacgtcgaatgcggcgaaggccatttctgccacgacaaccaaacctgctgcagggacaacaggcagggatgggcttgctgtccatatagacaaggggtgtgctgcgcagacaggagacattgctgccctgctgggttccggtgcgctgcaaggggaactaagtgcctgagacgggaggcacctcggtgggatgccccactgagggaccccgctttgagacagctcctg

SEQ ID NO: 5 - DNA, 1782 bp, synthetic construct
  source 1..1782: mol_type = other DNA
atgtggaccctggtgtcttgggtggccctgaccgccggtctggtggccggaaccagatgccccgatggccagttctgccccgtggcttgctgtctggaccccggcggagcatcttatagctgctgcagacctctgctggataagtggcctacaactctgtcccggcacctcggcggcccttgccaagtggacgcccactgtagtgccggacactcttgtatcttcaccgtatctggaaccagcagctgttgccctttccccgaggccgtggcctgtggcgacggtcatcactgctgccctcggggcttccactgttctgctgatggcagaagctgcttccagagaagcggcaacaacagcgtcggagccattcagtgtccggacagccagtttgagtgccctgacttcagcacatgctgcgtgatggtcgacggctcctggggctgctgccctatgccccaggccagctgctgcgaggacagagtgcactgctgccctcacggcgctttttgtgatctggtccacacaagatgcatcacaccaaccggcacacaccctctggccaagaaactgcctgcccagcggaccaaccgggccgttgccctgagcagcagcgtgatgtgcccagatgccagatcccggtgccctgacggcagcacctgttgtgaactgccttctggcaaatacggctgctgtcccatgcccaatgctacgtgctgctccgaccacctgcactgctgccctcaggacacagtgtgcgacctgatccaaagcaagtgcctgagcaaagagaacgccacaacagacctgctgaccaagctgcctgctcataccgtgggcgatgtgaagtgcgatatggaagtgtcctgccccgacggctacacctgttgtagactgcagagcggcgcctggggctgctgtcctttcacccaggccgtctgctgtgaggatcacatccactgttgccctgccggcttcacctgcgacacccagaagggcacctgcgagcagggcccccaccaggtgccctggatggaaaaggccccagctcatctgagcctgcctgaccctcaagctctgaagcgggacgtgccatgtgataacgtgtccagctgtccttctagcgatacctgttgccaactgaccagcggcgagtggggatgctgtcctatccccgaggccgtgtgctgcagcgaccaccagcactgctgtccccagggctacacctgtgtggccgagggccagtgccagagaggaagcgagatcgtggctggcctggaaaagatgcctgcccgacgggccagcctgagccaccccagagacatcggctgtgaccagcacaccagctgcccagtgggacagacctgctgccctagcctgggcggctcttgggcctgctgccagctgccccacgccgtgtgctgcgaggacagacagcactgctgccctgccggatacacctgcaacgtgaaggcccggagctgcgagaaggaagtggtgagcgctcaacctgccaccttcctggccagatcccctcatgtgggcgttaaggacgtggaatgcggggaaggccacttctgccacgacaaccagacatgctgcagagataatagacagggatgggcctgctgtccctacagacagggcgtgtgttgcgccgaccggagacactgttgcccagccggctttagatgcgccgcacggggcacaaaatgcctgagaagggaagcccctagatgggacgcccctttgagagatcctgccctgcgccagctgctctga

SEQ ID NO: 6 - DNA, 1782 bp, synthetic construct
  source 1..1782: mol_type = other DNA
atgtggacactggtgagctgggtggcccttacagccggcctggtggccggcacaagatgccctgatggacaattttgcccagtggcctgctgcctggaccctggcggagccagctacagctgctgcagacccctcctggataagtggcccacaaccctgtctagacacctgggcggaccttgccaggtggacgcccactgctctgccggccacagctgcatcttcaccgtgtctggaacatctagctgttgtcccttccctgaggctgttgcctgtggcgatggccaccactgctgtcctagaggcttccattgctctgccgatggtagatcctgcttccagagatcaggcaacaacagcgtgggagccatccagtgccctgatagccagttcgagtgcccagacttcagcacttgctgtgtgatggtcgatggctcttggggctgttgtcccatgccccaggcttcttgctgcgaagatagagtgcactgctgcccccacggcgccttctgtgacctggtccacaccagatgcatcacccccaccggcacacaccccctggcaaagaagctgcctgctcagagaaccaatagagctgtggccctgagctctagcgtgatgtgccctgatgccagatccagatgtcctgatggcagcacctgctgcgagctgccatctggcaaatatggctgttgtccaatgcctaacgccacctgctgttctgaccacctgcactgctgccctcaagacaccgtgtgcgacctgatccagagcaaatgcctctccaaggaaaacgccacaaccgacctgctgaccaagctgcctgcccacacagtgggcgatgtgaagtgtgatatggaagtgagctgccctgatggctacacctgttgcagactgcagtctggcgcctggggctgctgtcctttcacccaggccgtgtgttgcgaggaccacatccactgctgccctgctggatttacctgtgacacacagaagggcacctgtgaacagggccctcaccaggtgccatggatggaaaaggcccctgctcacctgagcctgccagaccctcaggccctgaagagggatgtcccctgcgacaatgtgagctcctgccctagctctgatacatgctgccagctgaccagcggagagtggggatgctgccccatccctgaggccgtgtgctgctccgaccaccaacattgttgcccccagggctatacctgtgtggccgagggccaatgtcaaagaggtagcgaaattgtggccggcctggagaagatgcctgccagaagagccagcctgagccaccctagagacatcggctgtgaccagcacaccagctgtcctgtgggccagacatgctgccctagcctgggcggcagctgggcctgctgccagctgcctcacgccgtctgttgtgaggatagacagcactgctgtcctgctggctacacatgcaatgtgaaagccaggtcctgtgagaaagaggtggtgagcgcccagcctgctacctttctggccagaagccctcatgtgggcgtgaaggatgtggaatgtggcgagggccatttctgtcacgacaaccagacctgttgtagagacaacagacagggatgggcttgctgcccatacagacagggcgtttgctgtgccgacagaagacactgctgccctgccggattcagatgcgctgccagagggaccaagtgtctgagaagagaagcccctagatgggacgcccctctgagggaccctgctctcagacagctgctgtga

SEQ ID NO: 7 - DNA, 1782 bp, synthetic construct
  source 1..1782: mol_type = other DNA
atgtggaccctggtgtcctgggtcgccctgaccgctggcctcgtggccggcacaagatgccccgacgggcagttctgtcctgtggcttgctgcctggaccccggcggcgctagctacagctgctgcagacccctgctggacaaatggcctaccacactgtcccggcatctcggcggcccctgtcaagtcgacgcccactgcagcgccggccacagctgcatcttcaccgtctccggcacaagcagctgctgccccttccctgaggccgtggcctgtggcgatgggcaccactgctgccctagaggcttccactgtagcgccgatggcagaagctgctttcagagaagcggcaacaacagcgtgggcgccattcagtgccccgacagccaattcgagtgccccgacttcagcacctgctgcgtgatggtcgatggcagctgggggtgctgccccatgccccaagctagctgctgtgaagacagagtgcattgctgcccccacggcgccttctgcgacctggtgcacacaagatgcatcacccccaccggcacccaccccctggccaagaagctgcccgcccaaagaaccaacagagccgtggccctgagcagcagcgtgatgtgccccgacgctagaagcagatgtcccgacggcagcacatgctgcgagctgcctagcggcaagtacggctgctgccccatgcccaatgctacctgctgcagcgatcatctgcactgttgcccccaagacaccgtgtgcgacctgattcagagcaaatgcctgtccaaggagaacgccaccaccgacctgctgaccaagctgcctgcccataccgtgggcgacgtgaagtgcgacatggaggtgagctgccccgacggctacacatgctgcagactgcagagcggcgcctgggggtgctgccctttcacccaagccgtgtgctgcgaggaccacatccactgttgccctgctggcttcacctgtgacacacagaagggcacctgcgagcaaggccctcaccaagtgccttggatggagaaggcccccgcccacctcagcctgcccgacccccaagccctgaagagagacgtgccctgcgacaacgtgagcagctgccctagctccgacacctgctgtcagctcacaagcggcgagtggggctgctgtcccatccccgaggctgtgtgctgcagcgaccaccaacactgctgcccccaaggctacacatgtgtcgccgaggggcagtgtcagagaggcagcgagatcgtggccggcctggagaagatgcccgctagaagagctagcctgagccaccctagagacatcggctgcgatcagcatacaagctgccccgtggggcagacctgctgcccttccctgggcggcagctgggcttgttgccaactgcctcacgccgtgtgttgcgaagacagacagcactgctgccccgccggctacacatgtaatgtgaaggctagaagctgcgagaaagaggtggtgagcgctcagcccgccaccttcctggctagaagcccccacgtgggcgtgaaggacgtggagtgcggcgagggccacttctgccacgacaatcagacctgttgtcgggacaacagacaaggctgggcttgctgcccctaccggcaaggcgtgtgctgcgccgacagaagacactgctgtcctgccggcttcagatgcgccgctagaggcaccaagtgcctgcggagagaggcccctagatgggacgcccccctgagagaccccgccctgagacagctgctgtga

SEQ ID NO: 8 - DNA, 1782 bp, synthetic construct
  source 1..1782: mol_type = other DNA
atgtggaccctggtgagctgggtggctctgacagctggccttgtggctggaactagatgtcccgatggacaattctgccccgtggcctgctgtctggatcccggaggcgctagctatagctgttgtagacccctgctggataagtggcccaccaccctgagcagacacctgggcggaccttgtcaagtggacgctcattgcagcgctggacacagctgcattttcaccgtgagcggcaccagcagctgctgcccttttcctgaagctgtggcctgcggggacggccaccattgttgtcctagaggatttcactgcagcgccgacggcagaagctgcttccaaagaagcggcaacaacagcgtgggcgccatccagtgtcccgacagccaatttgagtgccccgacttcagcacctgctgcgtgatggtggacggcagctggggatgttgtcctatgcctcaagccagctgctgtgaggacagagtgcactgctgcccccacggcgctttttgcgatctggtgcacaccagatgcatcacccccaccggcacacatcccctggctaaaaagctgcctgcccaaagaacaaacagagccgtggccctgagcagcagcgtgatgtgtcctgatgccagaagcagatgccccgacggcagcacatgttgtgagctgcccagcggcaaatacggctgctgccctatgcccaacgccacctgttgtagcgaccatctgcactgctgtccccaggacacagtgtgcgacctgatccagagcaagtgcctgagcaaggagaacgccaccaccgacctgctgaccaagctgcccgctcataccgtgggcgatgtgaagtgcgatatggaggtgagctgccccgacggctatacctgttgcagactgcaaagcggcgcctggggctgttgtccctttacccaagctgtgtgttgcgaggaccacatccactgctgccccgctggatttacctgcgacacacagaaaggcacctgcgagcagggaccccatcaggtgccttggatggaaaaagcccccgctcacctgagcctgcccgatcctcaagctcttaaaagagatgtgccctgcgataacgtgagcagctgccccagcagcgacacctgttgtcaactgaccagcggcgaatggggctgctgccctattcctgaggccgtgtgctgtagcgaccaccagcattgttgcccccagggctatacatgcgtggccgagggacaatgccagagaggaagcgaaattgtggctggcctggaaaagatgcccgccagaagagccagcctgagccaccctagagatatcggctgtgaccagcacaccagctgccccgttggacaaacatgttgtcctagcctgggcggaagctgggcctgttgtcaactgcctcacgccgtgtgttgcgaagatagacagcactgctgccccgccggatacacctgcaatgtgaaggccagaagctgcgagaaggaggtggtgagcgcccagcctgctacatttctggccagaagcccccatgtgggcgtgaaggatgtggagtgtggcgagggacatttctgccatgataaccagacctgctgcagagacaacagacagggctgggcctgctgcccctatagacaaggagtgtgttgcgctgacagaagacactgctgtcccgccggctttagatgcgccgctagaggcacaaagtgcctgagaagagaggcccccagatgggacgcccctcttagagatcctgctctgagacaactgctgtga

SEQ ID NO: 9 - DNA, 1782 bp, synthetic construct
  source 1..1782: mol_type = other DNA
atgtggaccctggtgagctgggtggccttaacagcagggctggtggctggaacgcggtgcccagatggtcagttctgccctgtggcctgctgcctggaccccggaggagccagctacagctgctgccgtccccttctggacaaatggcccacaacactgagcaggcatctgggtggcccctgccaggttgatgcccactgctctgccggccactcctgcatctttaccgtctcagggacttccagttgctgccccttcccagaggccgtggcatgcggggatggccatcactgctgcccacggggcttccactgcagtgcagacgggcgatcctgcttccaaagatcaggtaacaactccgtgggtgccatccagtgccctgatagtcagttcgaatgcccggacttctccacgtgctgtgttatggtcgatggctcctgggggtgctgccccatgccccaggcttcctgctgtgaagacagggtgcactgctgtccgcacggtgccttctgcgacctggttcacacccgctgcatcacacccacgggcacccaccccctggcaaagaagctccctgcccagaggactaacagggcagtggccttgtccagctcggtcatgtgtccggacgcacggtcccggtgccctgatggttctacctgctgtgagctgcccagtgggaagtatggctgctgcccaatgcccaacgccacctgctgctccgatcacctgcactgctgcccccaagacactgtgtgtgacctgatccagagtaagtgcctctccaaggagaacgctaccacggacctcctcactaagctgcctgcgcacacagtgggggatgtgaaatgtgacatggaggtgagctgcccagatggctatacctgctgccgtctacagtcgggggcctggggctgctgcccttttacccaggctgtgtgctgtgaggaccacatacactgctgtcccgcggggtttacgtgtgacacgcagaagggtacctgtgaacaggggccccaccaggtgccctggatggagaaggccccagctcacctcagcctgccagacccacaagccttgaagagagatgtcccctgtgataatgtcagcagctgtccctcctccgatacctgctgccaactcacgtctggggagtggggctgctgtccaatcccagaggctgtctgctgctcggaccaccagcactgctgcccccagggctacacgtgtgtagctgaggggcagtgtcagcgaggaagcgagatcgtggctggactggagaagatgcctgcccgccgggcttccttatcccaccccagagacatcggctgtgaccagcacaccagctgcccggtggggcagacctgctgcccgagcctgggtgggagctgggcctgctgccagttgccccatgctgtgtgctgcgaggatcgccagcactgctgcccggctggctacacctgcaacgtgaaggctcgatcctgcgagaaggaagtggtctctgcccagcctgccaccttcctggcccgtagccctcacgtgggtgtgaaggacgtggagtgtggggaaggacacttctgccatgataaccagacctgctgccgagacaaccgacagggctgggcctgctgtccctaccgccagggcgtctgttgtgctgatcggcgccactgctgtcctgctggcttccgctgcgcagccaggggtaccaagtgtttgcgcagggaggccccgcgctgggacgcccctttgagggacccagccttgagacagctgctgtga

SEQ ID NO: 10 - protein, 593 aa, synthetic construct
MWTLVSWVALTAGLVAGTRCPDGQFCPVACCLDPGGASYSCCRPLLDKWPTTLSRHLGGPCQVDAHCSAGHSCIFTVSGTSSCCPFPEAVACGDGHHCCPRGFHCSADGRSCFQRSGNNSVGAIQCPDSQFECPDFSTCCVMVDGSWGCCPMPQASCCEDRVHCCPHGAFCDLVHTRCITPTGTHPLAKKLPAQRTNRAVALSSSVMCPDARSRCPDGSTCCELPSGKYGCCPMPNATCCSDHLHCCPQDTVCDLIQSKCLSKENATTDLLTKLPAHTVGDVKCDMEVSCPDGYTCCRLQSGAWGCCPFTQAVCCEDHIHCCPAGFTCDTQKGTCEQGPHQVPWMEKAPAHLSLPDPQALKRDVPCDNVSSCPSSDTCCQLTSGEWGCCPIPEAVCCSDHQHCCPQGYTCVAEGQCQRGSEIVAGLEKMPARRASLSHPRDIGCDQHTSCPVGQTCCPSLGGSWACCQLPHAVCCEDRQHCCPAGYTCNVKARSCEKEVVSAQPATFLARSPHVGVKDVECGEGHFCHDNQTCCRDNRQGWACCPYRQGVCCADRRHCCPAGFRCAARGTKCLRREAPRWDAPLRDPALRQLL

SEQ ID NO: 11 - DNA, 561 bp, synthetic construct
  source 1..561: mol_type = other DNA
atggcaaagactgctatggcttataaggagaaaatgaaggaactctctatgctctctctcatttgttcctgcttctatcctgagcctcggaacattaatatctacacttacgatgatatggaggtaaagcaaattaacaagcgagcgagtgggcaggccttcgaacttatattgaagccaccctcccctatatccgaagcgccgaggacattggccagcccaaaaaagaaagacctttccttggaggagattcaaaagaagttggaggctgcggaggaaaggagaaagtctcaagaggcccaagtcttgaagcagctcgccgaaaagagggagcacgaaagggaggttctccagaaagctctcgaggaaaacaataacttttcaaaaatggcggaggaaaaactcatccttaagatggagcaaattaaagaaaatcgggaggctaacctcgccgcaataatcgaaaggttgcaagagaagctcgtcaagtttatttcctcagagcttaaagaatcaatagagtcccagtttttggagctccaaagagaaggcgagaagcag

SEQ ID NO: 12 - DNA, 561 bp, synthetic construct
  source 1..561: mol_type = other DNA
atggcaaagacagctatggcatataaggagaaaatgaaagaactgagcatgctgtctctgatatgttcatgtttttatcctgagccacgcaatattaacatttatacatacgacgacatggaggtcaaacaaataaataaaagagcttctggacaagcattcgaactcatactcaagccaccctcacccatttctgaagcccctaggactttggcatcaccaaaaaagaaagacctcagccttgaagagatacagaagaaactggaagctgcagaagagaggcgcaaatcccaggaggcccaagttctgaaacagctcgccgagaaacgggaacatgaaagggaagttctccagaaggctctggaggagaacaacaatttctctaagatggctgaggagaagttgatcttgaagatggaacagataaaggagaatagagaggcgaacctggccgccatcatagaacggctgcaggaaaagctggtgaagtttattagctccgaactcaaagaaagcatagaatcccagtttctggaactgcagcgggaaggtgaaaaacag

SEQ ID NO: 13 - DNA, 561 bp, synthetic construct
  source 1..561: mol_type = other DNA
atggctaagacagcaatggcttataaagagaagatgaaagagctcagcatgttgtccttgatttgctcttgtttctacccagaaccacggaacataaacatatatacatatgatgatatggaagtgaagcaaatcaataaaagggcctctggtcaagcgtttgaactgatcttgaagcctccctccccaattagcgaggccccaaggaccctcgcttctcccaaaaaaaaggacctctctctggaggagatccaaaagaagttggaggcagccgaggaaaggcgcaaatcccaggaagcgcaagtcctgaagcagctcgccgaaaagagggagcacgagcgggaggtcctccaaaaagcactcgaagaaaataataacttttctaagatggctgaggaaaaattgatactcaagatggagcagattaaagaaaatagagaagctaacctggcagccattatagagaggctccaggagaagttggtgaagtttatcagctctgaactgaaagagtcaatcgaatctcaatttcttgagctccagagggaaggagaaaagcaa

SEQ ID NO: 14 - DNA, 561 bp, synthetic construct
  source 1..561: mol_type = other DNA
atggccaagaccgctatggcctataaggaaaagatgaaggagctctccatgctgagcctcatttgtagctgcttctaccctgagccccggaacattaacatctacacctatgacgacatggaagtcaaacagataaataagcgggcatccggccaggccttcgaactgatcctcaagccaccttctcctatttccgaagctcccaggaccttggcctcccccaagaaaaaggatctgagcctcgaagagattcagaagaagctggaagctgctgaggagcggaggaagagccaggaggcgcaggtgctgaagcagctcgccgagaagagggagcatgaacgggaggtcttgcagaaggccctcgaagagaacaataacttctccaagatggctgaggaaaagctcatcctcaagatggagcaaatcaaagagaacagagaggccaacctggctgcaatcatcgagaggctccaggaaaagctcgtgaaattcatttctagcgagctcaaagaatctatcgagtcccagtttttggaactccagagggaaggcgaaaaacaa

SEQ ID NO: 15 - DNA, 564 bp, synthetic construct
  source 1..564: mol_type = other DNA
atggccaagaccgccatggcctacaaggagaagatgaaagagctgagcatgctgtctctgatctgcagctgtttctaccccgagcctcggaacatcaacatctatacatacgacgatatggaagtgaaacagatcaacaagcgggccagcggccaggccttcgagctcatcctgaaacctcctagccccatctctgaggcccctagaaccctggcctctccaaagaagaaggacctgtccctggaagagatccagaagaagctggaggctgctgaggaacgcagaaagtcccaggaggcccaggtcctgaagcagctggcagaaaaaagagagcacgagagagaggtgctgcagaaagccctggaagagaacaacaatttcagcaagatggccgaggaaaagctgattctgaagatggaacagatcaaggaaaatagagaagccaacctggccgctatcatcgagcggctgcaggagaagctggtgaagttcatcagcagcgagctgaaggaaagcatcgagagccaatttctggaactgcaaagagaaggcgagaagcagtga

SEQ ID NO: 16 - DNA, 564 bp, synthetic construct
  source 1..564: mol_type = other DNA
atggccaagaccgccatggcctataaggaaaagatgaaggaactgagcatgctgagcctgatctgtagctgcttctaccctgagcccagaaacatcaacatctacacctacgatgacatggaagtgaaacagatcaacaagagagccagcggccaggccttcgagctgatcctgaaacctccatctcctatcagcgaggcccctagaacactggctagccctaagaaaaaggatctgtctctggaagaaatccagaaaaagctggaagcagctgaggaaagaagaaagtcccaggaggcccaggtcctgaagcagctggccgagaagagagagcacgagagagaggtgctgcagaaggccctggaagagaacaacaatttcagcaagatggctgaagagaaactgattctgaagatggaacagatcaaggaaaatagagaggccaacctggccgccatcatcgagaggctgcaggagaagctcgtgaagttcatctccagcgagctgaaggagagcatcgagtctcaatttctggaacttcaaagagagggcgagaagcagtga

SEQ ID NO: 17 - DNA, 564 bp, synthetic construct
  source 1..564: mol_type = other DNA
atggccaagaccgccatggcctacaaggagaagatgaaggagctgagcatgctgagcctgatctgcagctgcttctaccctgagcctagaaacatcaacatctacacctacgatgacatggaggtgaagcagatcaacaagagagctagcggccaagccttcgagctgatcctgaagccccctagccccatcagcgaggcccctagaaccctggctagccccaagaaaaaggacctgagcctggaggagattcagaagaagctggaggccgccgaggagagaagaaagagccaagaggcccaagtgctgaagcagctggccgagaagagagagcacgagagagaggtgctgcagaaggccctggaggagaacaataacttcagcaagatggccgaggagaagctcatcctgaagatggagcagatcaaggagaacagagaggccaacctggccgccatcatcgagagactgcaagagaagctggtgaagttcatcagcagcgagctgaaggagagcatcgagagccaattcctggagctgcagagagagggcgagaagcagtga

SEQ ID NO: 18 - DNA, 564 bp, synthetic construct
  source 1..564: mol_type = other DNA
atggccaagaccgccatggcctacaaggagaagatgaaggagctgagcatgctgagcctgatctgcagctgcttctaccccgagcccagaaacatcaacatctacacctacgatgacatggaggtgaagcagatcaacaagagagccagcggccaggccttcgagctgattctgaaacctcccagccccatcagcgaggcccctagaacactggctagccctaagaaaaaggacctgagcctggaggagatccagaagaagctggaggccgccgaggagagaagaaagagccaggaggcccaggtgctgaagcagctggctgagaaaagagagcacgagagagaggtgctgcagaaggccctggaggagaacaacaacttcagcaagatggccgaggagaagctgatcctgaagatggagcagatcaaggagaacagagaggccaacctggccgccatcatcgagagactgcaggagaagctggtgaagttcatcagcagcgagctgaaggagagcatcgagagccagttcctggagctgcagagagagggcgagaagcagtga

SEQ ID NO: 19 - DNA, 564 bp, synthetic construct
  source 1..564: mol_type = other DNA
atggctaaaacagcaatggcctacaaggaaaaaatgaaggagctgtccatgctgtcactgatctgctcttgcttttacccggaacctcgcaacatcaacatctatacttacgatgatatggaagtgaagcaaatcaacaaacgtgcctctggccaggcttttgagctgatcttgaagccaccatctcctatctcagaagccccacgaactttagcttctccaaagaagaaagacctgtccctggaggagatccagaagaaactggaggctgcagaggaaagaagaaagtctcaggaggcccaggtgctgaaacaattggcagagaagagggaacacgagcgagaagtccttcagaaggctttggaggagaacaacaacttcagcaagatggcggaggaaaagctgatcctgaaaatggaacaaattaaggaaaaccgtgaggctaatctagctgctattattgaacgtctgcaggaaaagctggtcaagtttatttcttctgaactaaaagaatctatagagtctcaatttctggagcttcagagggaaggagagaagcaatga

SEQ ID NO: 20 - protein, 187 aa, synthetic construct
MAKTAMAYKEKMKELSMLSLICSCFYPEPRNINIYTYDDMEVKQINKRASGQAFELILKPPSPISEAPRTLASPKKKDLSLEEIQKKLEAAEERRKSQEAQVLKQLAEKREHEREVLQKALEENNNFSKMAEEKLILKMEQIKENREANLAAIIERLQEKLVKFISSELKESIESQFLELQREGEKQ

SEQ ID NO: 21 - DNA, 2400 bp, synthetic construct
  source 1..2400: mol_type = other DNA
atgtggaccctcgtttcttgggtcgccctgacagccgggctggttgccggcactcggtgccctgacggacagttttgccccgtggcttgttgtcttgatccagggggggccagttactcttgttgtaggccattgcttgacaaatggccaaccactctttccaggcacctgggaggaccttgtcaggtggacgctcactgcagcgctggccacagctgtattttcacagtgtctgggacatcatcctgttgtcctttccccgaagcggtggcttgtggcgatggccatcattgctgtcctcggggatttcattgtagcgcagacggacgcagctgctttcaacggtctggaaacaattccgtcggggccatacaatgcccagattctcagtttgaatgccccgatttctctacctgttgcgtgatggtggacggctcctggggctgctgtcctatgccccaggcctcctgctgcgaggacagggtgcattgttgtcctcatggggccttctgtgacctcgttcacacaaggtgtattacaccaacaggcactcaccctctggccaagaaacttcctgcccagagaaccaatagggcagttgctctgagctcttcagtgatgtgccctgatgcccgctctcgctgtcctgacggcagcacatgctgtgaactcccttcaggcaagtatggatgttgtcctatgcccaatgcaacttgctgttccgaccacctccattgctgtcctcaagatacagtctgtgacctcatacagtcaaagtgcctttccaaagaaaacgccacaaccgatcttttgacaaagcttcccgcccacacagttggcgatgtgaagtgtgatatggaggtgtcctgccctgatggatatacttgctgccgcctgcaaagtggagcatggggctgctgtccattcacccaagccgtgtgctgcgaggatcatatccactgttgccctgctgggtttacgtgcgatactcaaaaaggcacctgcgaacagggaccacaccaagttccctggatggagaaagctccagcacatctttctttgccggatccacaggccctcaagcgggatgtcccatgcgataatgtgtccagctgcccgagttctgacacctgctgtcagttgacttctggagagtggggatgttgcccaattccagaggctgtttgctgttcagatcatcaacactgttgcccacagggttatacctgtgtcgctgaaggccaatgtcaacggggctctgagatcgttgcgggcctcgagaagatgcccgcccggcgcgcctctttgagccacccaagagacatcggatgcgaccagcacacctcatgtcctgtcggacagacttgttgtccttccctgggtggatcttgggcgtgttgtcaactcccccatgcagtatgctgcgaagataggcaacattgctgcccagctgggtatacctgtaatgtcaaggctagatcatgcgaaaaagaggttgtttctgctcagccagccacatttctcgcacggagccctcatgtgggggttaaagacgtcgagtgtggcgaagggcatttctgtcacgacaatcaaacatgttgtcgggataaccggcagggatgggcatgttgtccctatcggcaaggcgtgtgctgtgccgacagacggcattgctgtccagctgggttcagatgtgcagcaagagggactaaatgtcttcggagggaggccccccggtgggatgcaccccttcgggaccccgccctgagacagctgctggccactaacttcagccttttgaagcaggccggagatgtcgaagaaaaccctggacctatggcaaagactgctatggcttataaggagaaaatgaaggaactctctatgctctctctcatttgttcctgcttctatcctgagcctcggaacattaatatctacacttacgatgatatggaggtaaagcaaattaacaagcgagcgagtgggcaggccttcgaacttatattgaagccaccctcccctatatccgaagcgccgaggacattggccagcccaaaaaagaaagacctttccttggaggagattcaaaagaagttggaggctgcggaggaaaggagaaagtctcaagaggcccaagtcttgaagcagctcgccgaaaagagggagcacgaaagggaggttctccagaaagctctcgaggaaaacaataacttttcaaaaatggcggaggaaaaactcatccttaagatggagcaaattaaagaaaatcgggaggctaacctcgccgcaataatcgaaaggttgcaagagaagctcgtcaagtttatttcctcagagcttaaagaatcaatagagtcccagtttttggagctccaaagagaaggcgagaagcagtga

SEQ ID NO: 22 - DNA, 2400 bp, synthetic construct
  source 1..2400: mol_type = other DNA
atggcaaagacagctatggcatataaggagaaaatgaaagaactgagcatgctgtctctgatatgttcatgtttttatcctgagccacgcaatattaacatttatacatacgacgacatggaggtcaaacaaataaataaaagagcttctggacaagcattcgaactcatactcaagccaccctcacccatttctgaagcccctaggactttggcatcaccaaaaaagaaagacctcagccttgaagagatacagaagaaactggaagctgcagaagagaggcgcaaatcccaggaggcccaagttctgaaacagctcgccgagaaacgggaacatgaaagggaagttctccagaaggctctggaggagaacaacaatttctctaagatggctgaggagaagttgatcttgaagatggaacagataaaggagaatagagaggcgaacctggccgccatcatagaacggctgcaggaaaagctggtgaagtttattagctccgaactcaaagaaagcatagaatcccagtttctggaactgcagcgggaaggtgaaaaacaggctaccaacttttccttgctgaagcaggcaggagatgttgaagaaaatcctggacccatgtggacactcgttagttgggttgccttgaccgctggattggtcgccggaacacgctgtcctgatggacagttttgtccagtcgcatgctgtttggatcctgggggcgctagttacagctgctgcaggccactgctcgataaatggccgactacattgtcacgccatttgggagggccttgccaagtcgatgctcactgttccgccggtcattcttgcattttcacagtttccggcactagctcatgctgcccgtttccggaagccgttgcctgtggcgatgggcatcattgctgtcctcggggctttcactgcagcgccgacggaaggtcctgcttccagaggtcagggaacaatagcgttggagcgattcaatgccctgactctcaatttgagtgtccagattttagcacctgctgtgtcatggtcgatggatcttggggctgttgccccatgcctcaagcgtcatgttgcgaggatcgcgtccactgttgtccacacggagccttttgtgacctggtacatactcggtgcatcacaccaaccgggacgcaccctttggccaaaaagcttcccgctcagagaacaaatcgggccgtcgcactcagtagttccgtaatgtgtcccgacgctcgctctcggtgcccagacggctctacatgctgcgagctgccgtcagggaagtatggctgctgtcctatgcccaacgctacttgttgttcagaccacctccattgctgtcctcaggataccgtttgtgatctcattcaaagtaagtgtctgtccaaagaaaatgctactaccgacttgctgactaaacttccagctcatacagtgggggatgtgaaatgcgacatggaagtctcttgtccagacgggtatacatgctgtaggctgcagtcaggggcctggggatgctgtccttttactcaagctgtttgttgtgaagaccatatacactgctgccccgcaggctttacctgtgatacccagaaaggaacatgtgagcagggtccacatcaagtgccttggatggagaaagcccccgctcacctttctcttcctgacccgcaggcgttgaagagggacgtgccttgtgacaatgtttcatcatgtccaagctctgacacctgttgtcaactgacatctggcgagtggggatgttgcccaatcccagaagccgtctgttgttctgatcaccaacactgttgcccacagggctatacctgcgtggccgaaggtcaatgccaaagagggagcgagattgttgcgggactggaaaaaatgcccgctcggagggccagcctgtcccacccacgagacataggatgtgaccagcatacctcttgtccggttggtcagacgtgctgcccaagcttggggggatcttgggcatgttgtcaactgccgcacgcagtttgttgcgaagacagacaacactgctgtcccgcgggctacacttgtaacgttaaggcaagaagctgcgaaaaagaggtggtttctgcccagccagcaaccttcctcgctagatcccctcacgttggcgtgaaggacgtggagtgcggcgagggacatttctgtcacgataaccaaacctgctgtcgcgacaaccgccagggttgggcctgctgcccttatcgccagggagtgtgctgtgccgatcggcgccattgttgtcctgcagggtttagatgcgctgctaggggtactaaatgtctgagaagggaagctccgagatgggatgcccctttgagagatccagctctgagacaattgctctga

SEQ ID NO: 23 - DNA, 2400 bp, synthetic construct
  source 1..2400: mol_type = other DNA
atgtggacacttgtttcatgggttgcactcaccgctggactggtcgctggaaccagatgtccagacggacagttttgcccagtggcatgctgtctcgatcctggcggagctagctactcttgctgcagaccactgttggataagtggccaacaacactgagcagacatctgggcggcccatgccaagtggatgctcattgtagcgccggccactcctgcattttcactgtgtccggtacctcctcatgctgcccattccctgaagccgtcgcatgcggagatgggcatcactgttgccccagaggttttcactgcagcgctgatgggcggtcttgcttccaaaggtccggaaacaatagcgttggggcgattcagtgccctgactcccaatttgaatgccctgacttttcaacctgttgtgtaatggtcgatggcagctggggctgctgccccatgccacaggcttcttgttgcgaagaccgcgtgcattgttgccctcatggagcgttttgtgacctcgtgcacacgaggtgcattactcccacaggcactcatccactggctaagaaattgcctgcccagagaacaaacagggcagtagccttgagctcttcagtaatgtgtccggacgccagatcaaggtgccccgatggatctacttgctgcgagctcccctccggcaagtacggatgttgtccaatgccaaatgctacttgctgtagtgaccacctgcactgttgtccacaggatacggtctgtgacttgatacagtctaagtgcctgtctaaggaaaatgcaacaaccgatttgcttacaaaactgccagcccataccgtaggagatgtcaagtgcgacatggaagttagttgtcccgacggctatacgtgttgtcgactgcaatccggagcctggggttgttgcccgtttactcaggctgtatgttgcgaggaccacatccactgttgtcccgcggggttcacctgtgacacccagaaaggtacctgcgagcaggggcctcatcaggtaccgtggatggaaaaagctcctgcacacttgtcactgccggatcctcaggcgctcaagcgggatgtcccttgcgacaatgtaagctcctgtccgtctagtgatacgtgttgccaacttacctccggggaatggggctgttgccctataccagaagctgtctgttgctctgatcatcaacattgctgcccccagggttacacatgtgtagcagagggtcagtgtcaaagagggtcagaaattgtagctggcttggaaaaaatgcccgcaaggcgcgcatccctgtctcaccctcgggatataggatgcgatcagcacacctcttgtccagtgggtcagacttgttgtcccagcttggggggcagctgggcttgttgtcaattgcctcacgcagtttgctgtgaagaccgccaacattgctgtcctgctggatacacatgcaatgtcaaggctaggagctgtgaaaaagaggtggtgtctgcacagcccgcaacctttcttgcacgctctcctcatgtcggagtgaaagatgttgaatgcggagaaggacacttttgccatgacaatcagacttgttgcagggacaacagacaaggatgggcttgttgcccttatcgccaaggagtatgctgtgcggatagaagacattgttgcccggccgggtttaggtgtgctgctagaggcaccaaatgtctgaggcgagaagcacctagatgggacgctccactgcgggaccctgccttgagacaactcctggctactaacttcagcctgctgaagcaggctggagacgtggaggagaaccctggacctatggctaagacagcaatggcttataaagagaagatgaaagagctcagcatgttgtccttgatttgctcttgtttctacccagaaccacggaacataaacatatatacatatgatgatatggaagtgaagcaaatcaataaaagggcctctggtcaagcgtttgaactgatcttgaagcctccctccccaattagcgaggccccaaggaccctcgcttctcccaaaaaaaaggacctctctctggaggagatccaaaagaagttggaggcagccgaggaaaggcgcaaatcccaggaagcgcaagtcctgaagcagctcgccgaaaagagggagcacgagcgggaggtcctccaaaaagcactcgaagaaaataataacttttctaagatggctgaggaaaaattgatactcaagatggagcagattaaagaaaatagagaagctaacctggcagccattatagagaggctccaggagaagttggtgaagtttatcagctctgaactgaaagagtcaatcgaatctcaatttcttgagctccagagggaaggagaaaagcaatga

SEQ ID NO: 24 - DNA, 2400 bp, synthetic construct
  source 1..2400: mol_type = other DNA
atggctaagacagcaatggcttataaagagaagatgaaagagctcagcatgttgtccttgatttgctcttgtttctacccagaaccacggaacataaacatatatacatatgatgatatggaagtgaagcaaatcaataaaagggcctctggtcaagcgtttgaactgatcttgaagcctccctccccaattagcgaggccccaaggaccctcgcttctcccaaaaaaaaggacctctctctggaggagatccaaaagaagttggaggcagccgaggaaaggcgcaaatcccaggaagcgcaagtcctgaagcagctcgccgaaaagagggagcacgagcgggaggtcctccaaaaagcactcgaagaaaataataacttttctaagatggctgaggaaaaattgatactcaagatggagcagattaaagaaaatagagaagctaacctggcagccattatagagaggctccaggagaagttggtgaagtttatcagctctgaactgaaagagtcaatcgaatctcaatttcttgagctccagagggaaggagaaaagcaagctactaacttcagcctgctgaagcaggctggagacgtggaggagaaccctggacctatgtggacacttgtttcatgggttgcactcaccgctggactggtcgctggaaccagatgtccagacggacagttttgcccagtggcatgctgtctcgatcctggcggagctagctactcttgctgcagaccactgttggataagtggccaacaacactgagcagacatctgggcggcccatgccaagtggatgctcattgtagcgccggccactcctgcattttcactgtgtccggtacctcctcatgctgcccattccctgaagccgtcgcatgcggagatgggcatcactgttgccccagaggttttcactgcagcgctgatgggcggtcttgcttccaaaggtccggaaacaatagcgttggggcgattcagtgccctgactcccaatttgaatgccctgacttttcaacctgttgtgtaatggtcgatggcagctggggctgctgccccatgccacaggcttcttgttgcgaagaccgcgtgcattgttgccctcatggagcgttttgtgacctcgtgcacacgaggtgcattactcccacaggcactcatccactggctaagaaattgcctgcccagagaacaaacagggcagtagccttgagctcttcagtaatgtgtccggacgccagatcaaggtgccccgatggatctacttgctgcgagctcccctccggcaagtacggatgttgtccaatgccaaatgctacttgctgtagtgaccacctgcactgttgtccacaggatacggtctgtgacttgatacagtctaagtgcctgtctaaggaaaatgcaacaaccgatttgcttacaaaactgccagcccataccgtaggagatgtcaagtgcgacatggaagttagttgtcccgacggctatacgtgttgtcgactgcaatccggagcctggggttgttgcccgtttactcaggctgtatgttgcgaggaccacatccactgttgtcccgcggggttcacctgtgacacccagaaaggtacctgcgagcaggggcctcatcaggtaccgtggatggaaaaagctcctgcacacttgtcactgccggatcctcaggcgctcaagcgggatgtcccttgcgacaatgtaagctcctgtccgtctagtgatacgtgttgccaacttacctccggggaatggggctgttgccctataccagaagctgtctgttgctctgatcatcaacattgctgcccccagggttacacatgtgtagcagagggtcagtgtcaaagagggtcagaaattgtagctggcttggaaaaaatgcccgcaaggcgcgcatccctgtctcaccctcgggatataggatgcgatcagcacacctcttgtccagtgggtcagacttgttgtcccagcttggggggcagctgggcttgttgtcaattgcctcacgcagtttgctgtgaagaccgccaacattgctgtcctgctggatacacatgcaatgtcaaggctaggagctgtgaaaaagaggtggtgtctgcacagcccgcaacctttcttgcacgctctcctcatgtcggagtgaaagatgttgaatgcggagaaggacacttttgccatgacaatcagacttgttgcagggacaacagacaaggatgggcttgttgcccttatcgccaaggagtatgctgtgcggatagaagacattgttgcccggccgggtttaggtgtgctgctagaggcaccaaatgtctgaggcgagaagcacctagatgggacgctccactgcgggaccctgccttgagacaactcctgtga

SEQ ID NO: 25 - DNA, 2400 bp, synthetic construct
  source 1..2400: mol_type = other DNA
atgtggaccctcgtgtcctgggtggcactgacagccgggttggtggctggaactagatgcccagacggacaattctgtcccgtggcttgttgcttggaccctggaggcgcatcctatagctgttgcaggcccttgctggacaaatggccaaccacactgtctcggcatttgggcggcccatgccaggtggatgctcattgcagcgccggccacagctgcatcttcaccgtctccggaacaagcagctgttgtccatttccagaagcggtcgcttgtggcgatggacatcactgttgtcctcgcggatttcactgctctgccgatggaaggtcctgctttcaacggagcggaaataattctgtcggggccattcaatgtcccgactcccagttcgagtgccctgattttagcacatgttgtgtcatggtcgatggcagctggggatgctgcccaatgccacaggcatcctgctgcgaagatagggtacattgttgccctcatggagccttttgtgatctcgttcatacaaggtgtattactccaactggcacacatcccctcgccaagaaattgcctgcacagagaaccaaccgggctgtggccctctcttcctctgttatgtgtccagacgcacggtctagatgtcccgatggatctacatgttgcgaactgcctagcggaaagtatgggtgctgtcccatgcctaacgctacgtgctgctctgatcatttgcactgctgtccacaggatacagtgtgtgatctgattcaaagcaaatgcctgtccaaagaaaatgcaacaaccgatctcttgactaaactccctgcccacaccgtgggcgacgtaaagtgtgacatggaggtctcttgcccagacggctatacatgctgtcggctgcagtctggagcctggggctgttgccccttcactcaggccgtctgttgcgaggatcatatccattgttgtccagcaggatttacttgcgacacccagaaaggcacctgcgagcaaggcccacaccaagtgccttggatggagaaggccccagctcacctcagcctgcctgaccctcaggcactcaagagggatgtgccatgtgacaatgtttcttcctgcccttccagcgatacatgttgtcaattgacatctggcgaatggggatgttgtcccatcccagaggctgtgtgctgctccgatcaccagcactgttgtccacaggggtatacatgtgttgctgaaggccaatgtcaacggggctccgaaatcgtggcaggactcgaaaaaatgcctgcaaggagagctagcctctctcatccacgcgatatcggatgcgaccaacatacatcctgcccagtcggccaaacttgctgtccctcacttggaggttcctgggcttgctgccagctgccccatgccgtgtgctgcgaagatagacagcactgttgccctgccgggtacacctgcaatgtcaaggccagatcatgcgaaaaagaggtggtcagcgcacagccagctacattcttggccagatccccacatgtgggcgtgaaagacgtcgaatgcggcgaaggccatttctgccacgacaaccaaacctgctgcagggacaacaggcagggatgggcttgctgtccatatagacaaggggtgtgctgcgcagacaggagacattgctgccctgctgggttccggtgcgctgcaaggggaactaagtgcctgagacgggaggcacctcggtgggatgccccactgagggaccccgctttgagacagctcctggctactaacttcagcctgctgaagcaggctggagacgtggaggagaaccctggacctatggccaagaccgctatggcctataaggaaaagatgaaggagctctccatgctgagcctcatttgtagctgcttctaccctgagccccggaacattaacatctacacctatgacgacatggaagtcaaacagataaataagcgggcatccggccaggccttcgaactgatcctcaagccaccttctcctatttccgaagctcccaggaccttggcctcccccaagaaaaaggatctgagcctcgaagagattcagaagaagctggaagctgctgaggagcggaggaagagccaggaggcgcaggtgctgaagcagctcgccgagaagagggagcatgaacgggaggtcttgcagaaggccctcgaagagaacaataacttctccaagatggctgaggaaaagctcatcctcaagatggagcaaatcaaagagaacagagaggccaacctggctgcaatcatcgagaggctccaggaaaagctcgtgaaattcatttctagcgagctcaaagaatctatcgagtcccagtttttggaactccagagggaaggcgaaaaacaatga

SEQ ID NO: 26 - DNA, 2400 bp, synthetic construct
  source 1..2400: mol_type = other DNA
atggccaagaccgctatggcctataaggaaaagatgaaggagctctccatgctgagcctcatttgtagctgcttctaccctgagccccggaacattaacatctacacctatgacgacatggaagtcaaacagataaataagcgggcatccggccaggccttcgaactgatcctcaagccaccttctcctatttccgaagctcccaggaccttggcctcccccaagaaaaaggatctgagcctcgaagagattcagaagaagctggaagctgctgaggagcggaggaagagccaggaggcgcaggtgctgaagcagctcgccgagaagagggagcatgaacgggaggtcttgcagaaggccctcgaagagaacaataacttctccaagatggctgaggaaaagctcatcctcaagatggagcaaatcaaagagaacagagaggccaacctggctgcaatcatcgagaggctccaggaaaagctcgtgaaattcatttctagcgagctcaaagaatctatcgagtcccagtttttggaactccagagggaaggcgaaaaacaagctactaacttcagcctgctgaagcaggctggagacgtggaggagaaccctggacctatgtggaccctcgtgtcctgggtggcactgacagccgggttggtggctggaactagatgcccagacggacaattctgtcccgtggcttgttgcttggaccctggaggcgcatcctatagctgttgcaggcccttgctggacaaatggccaaccacactgtctcggcatttgggcggcccatgccaggtggatgctcattgcagcgccggccacagctgcatcttcaccgtctccggaacaagcagctgttgtccatttccagaagcggtcgcttgtggcgatggacatcactgttgtcctcgcggatttcactgctctgccgatggaaggtcctgctttcaacggagcggaaataattctgtcggggccattcaatgtcccgactcccagttcgagtgccctgattttagcacatgttgtgtcatggtcgatggcagctggggatgctgcccaatgccacaggcatcctgctgcgaagatagggtacattgttgccctcatggagccttttgtgatctcgttcatacaaggtgtattactccaactggcacacatcccctcgccaagaaattgcctgcacagagaaccaaccgggctgtggccctctcttcctctgttatgtgtccagacgcacggtctagatgtcccgatggatctacatgttgcgaactgcctagcggaaagtatgggtgctgtcccatgcctaacgctacgtgctgctctgatcatttgcactgctgtccacaggatacagtgtgtgatctgattcaaagcaaatgcctgtccaaagaaaatgcaacaaccgatctcttgactaaactccctgcccacaccgtgggcgacgtaaagtgtgacatggaggtctcttgcccagacggctatacatgctgtcggctgcagtctggagcctggggctgttgccccttcactcaggccgtctgttgcgaggatcatatccattgttgtccagcaggatttacttgcgacacccagaaaggcacctgcgagcaaggcccacaccaagtgccttggatggagaaggccccagctcacctcagcctgcctgaccctcaggcactcaagagggatgtgccatgtgacaatgtttcttcctgcccttccagcgatacatgttgtcaattgacatctggcgaatggggatgttgtcccatcccagaggctgtgtgctgctccgatcaccagcactgttgtccacaggggtatacatgtgttgctgaaggccaatgtcaacggggctccgaaatcgtggcaggactcgaaaaaatgcctgcaaggagagctagcctctctcatccacgcgatatcggatgcgaccaacatacatcctgcccagtcggccaaacttgctgtccctcacttggaggttcctgggcttgctgccagctgccccatgccgtgtgctgcgaagatagacagcactgttgccctgccgggtacacctgcaatgtcaaggccagatcatgcgaaaaagaggtggtcagcgcacagccagctacattcttggccagatccccacatgtgggcgtgaaagacgtcgaatgcggcgaaggccatttctgccacgacaaccaaacctgctgcagggacaacaggcagggatgggcttgctgtccatatagacaaggggtgtgctgcgcagacaggagacattgctgccctgctgggttccggtgcgctgcaaggggaactaagtgcctgagacgggaggcacctcggtgggatgccccactgagggaccccgctttgagacagctcctgtga

SEQ ID NO: 27 - DNA, 441 bp, synthetic construct
  source 1..441: mol_type = other DNA
gggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcag

SEQ ID NO: 28 - DNA, 122 bp, synthetic construct
  source 1..122: mol_type = other DNA
aacttgtttattgcagcttataatggttacaaataaagcaatagcatcacaaatttcacaaataaagcatttttttcactgcattctagttgtggtttgtccaaactcatcaatgtatctta

SEQ ID NO: 29 - DNA, 477 bp, synthetic construct
  source 1..477: mol_type = other DNA
gggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 30 - DNA, 589 bp, synthetic construct
  source 1..589: mol_type = other DNA
aatcaacctctggattacaaaatttgtgaaagattgactggtattcttaactatgttgctccttttaccctatgtggataggctgctttaatgcctttgtatcatgctattgcttccagtatggctttcattttctcctccttgtataaatcctggttgctgtctctttatgaggagttgtggccagttgtcaggcaacctggcctggtgtgcactgtgtttgctgaggcaacccccactggttggggcattgccaccacctgtcagctcctttcctggactttccctttccccctccctattgccagggcagaactcatcactgcctgccttgcctgctgctggacaggggctgggctgttgggcactgacaattccctggtgttgtcagggaaatcatggtcctttccttggctgctggcctgtgttgccacctggattctgggcaggaggtccttctgctaggtcccttgggccctcaatccagaggaccttccttccagcagcctgctgcaggctctgcagcctcttcagcttcttggccttggccctcagaggagtgggatctccctttgggcctcctccctgc

SEQ ID NO: 31 - DNA, 57 bp, synthetic construct
  source 1..57: mol_type = other DNA
gccactaacttcagccttttgaagcaggccggagatgtcgaagaaaaccctggacct

SEQ ID NO: 32 - DNA, 57 bp, synthetic construct
  source 1..57: mol_type = other DNA
gctaccaacttttccttgctgaagcaggcaggagatgttgaagaaaatcctggaccc

SEQ ID NO: 33 - DNA, 57 bp, synthetic construct
  source 1..57: mol_type = other DNA
gctactaacttcagcctgctgaagcaggctggagacgtggaggagaaccctggacct

SEQ ID NO: 34 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 35 - protein, 7 aa, synthetic construct
GVFVLPN

SEQ ID NO: 36 - protein, 7 aa, synthetic construct
GNYRGNP

SEQ ID NO: 37 - protein, 7 aa, synthetic construct
CSSRRSK

SEQ ID NO: 38 - protein, 7 aa, synthetic construct
FRHGPPS

SEQ ID NO: 39 - protein, 7 aa, synthetic construct
RFKTGYP

SEQ ID NO: 40 - protein, 7 aa, synthetic construct
GKHPAKL

SEQ ID NO: 41 - protein, 7 aa, synthetic construct
NRGRSGE

SEQ ID NO: 42 - protein, 7 aa, synthetic construct
REPRVGP

SEQ ID NO: 43 - protein, 7 aa, synthetic construct
VTFSHAQ

SEQ ID NO: 44 - protein, 7 aa, synthetic construct
DSRLTGR

SEQ ID NO: 45 - protein, 7 aa, synthetic construct
EPIARPL

SEQ ID NO: 46 - protein, 7 aa, synthetic construct
SSSWRPK

SEQ ID NO: 47 - protein, 7 aa, synthetic construct
DLRDVLG

SEQ ID NO: 48 - protein, 7 aa, synthetic construct
PYRSALW

SEQ ID NO: 49 - protein, 7 aa, synthetic construct
LKPYHLE

SEQ ID NO: 50 - protein, 7 aa, synthetic construct
GNKPNVD

SEQ ID NO: 51 - protein, 7 aa, synthetic construct
WTAVLVQ

SEQ ID NO: 52 - protein, 7 aa, synthetic construct
HTESTYG

SEQ ID NO: 53 - protein, 7 aa, synthetic construct
PDEKMTK

SEQ ID NO: 54 - protein, 7 aa, synthetic construct
QPWQQWQ

SEQ ID NO: 55 - protein, 7 aa, synthetic construct
LMLRPYM

SEQ ID NO: 56 - protein, 7 aa, synthetic construct
WRNQQVG

SEQ ID NO: 57 - protein, 7 aa, synthetic construct
VGRLARA

SEQ ID NO: 58 - protein, 7 aa, synthetic construct
LRRSRMS

SEQ ID NO: 59 - protein, 7 aa, synthetic construct
ILHAARA

SEQ ID NO: 60 - protein, 7 aa, synthetic construct
AGHTTKV

SEQ ID NO: 61 - protein, 7 aa, synthetic construct
AGDEWRP

SEQ ID NO: 62 - protein, 7 aa, synthetic construct
VTKKQET

SEQ ID NO: 63 - protein, 7 aa, synthetic construct
RPMSTCS

SEQ ID NO: 64 - protein, 7 aa, synthetic construct
RQRRANK

SEQ ID NO: 65 - protein, 7 aa, synthetic construct
HLETARQ

SEQ ID NO: 66 - protein, 7 aa, synthetic construct
LSDAKMG

SEQ ID NO: 67 - protein, 7 aa, synthetic construct
TRITSVY

SEQ ID NO: 68 - protein, 7 aa, synthetic construct
LWIESRP

SEQ ID NO: 69 - protein, 7 aa, synthetic construct
AKGDMCN

SEQ ID NO: 70 - protein, 7 aa, synthetic construct
VPDLKDC

SEQ ID NO: 71 - protein, 7 aa, synthetic construct
VQSESHG

SEQ ID NO: 72 - protein, 7 aa, synthetic construct
FFLDRPR

SEQ ID NO: 73 - protein, 7 aa, synthetic construct
VLTATID

SEQ ID NO: 74 - protein, 7 aa, synthetic construct
KGGAMCC

SEQ ID NO: 75 - protein, 7 aa, synthetic construct
IWHVRYE

SEQ ID NO: 76 - protein, 7 aa, synthetic construct
TGEHATT

SEQ ID NO: 77 - protein, 7 aa, synthetic construct
DKSTQPC

SEQ ID NO: 78 - protein, 7 aa, synthetic construct
PAVVIAN

SEQ ID NO: 79 - protein, 7 aa, synthetic construct
VTGDYGM

SEQ ID NO: 80 - protein, 7 aa, synthetic construct
EWSSKKT

SEQ ID NO: 81 - protein, 7 aa, synthetic construct
NRGVSIE

SEQ ID NO: 82 - protein, 7 aa, synthetic construct
PTDRQWP

SEQ ID NO: 83 - protein, 7 aa, synthetic construct
ISASYAR

SEQ ID NO: 84 - protein, 7 aa, synthetic construct
GPCSLPG

SEQ ID NO: 85 - protein, 7 aa, synthetic construct
DYAAPCQ

SEQ ID NO: 86 - protein, 7 aa, synthetic construct
GTREVEW

SEQ ID NO: 87 - protein, 7 aa, synthetic construct
RNVSPAR

SEQ ID NO: 88 - protein, 7 aa, synthetic construct
TGLGWTG

SEQ ID NO: 89 - protein, 7 aa, synthetic construct
SFERTDK

SEQ ID NO: 90 - protein, 7 aa, synthetic construct
QLDGDRS

SEQ ID NO: 91 - protein, 7 aa, synthetic construct
WWEPTTT

SEQ ID NO: 92 - protein, 7 aa, synthetic construct
DILTNYR

SEQ ID NO: 93 - protein, 7 aa, synthetic construct
HSYDRTS

SEQ ID NO: 94 - protein, 7 aa, synthetic construct
LPSAQLM

(19) United States
(12) Patent Application Publication
(10) Pub. No.: US 2026/0091134 A1
(43) Pub. Date: Apr. 2, 2026

(54) RECOMBINANT AAV VECTORS FOR TREATING PROTEINOPATHIES IN CENTRAL NERVOUS SYSTEM

(71) Applicant: SHANGHAI VITALGEN BIOPHARMA CO., LTD., Shanghai (CN)

(72) Inventors: Yezheng TAO, Shanghai (CN); Shin-Shay TIAN, Shanghai (CN); Xiaoping ZHAO, Shanghai (CN)

(73) Assignee: SHANGHAI VITALGEN BIOPHARMA CO., LTD., Shanghai (CN)

(21) Appl. No.: 19/401,460

(22) Filed: Nov. 26, 2025

Related U.S. Application Data
(63) Continuation of application No. PCT/CN2024/095067, filed on May 24, 2024.

(30) Foreign Application Priority Data
May 26, 2023 (WO) PCT/CN2023/096582

(51) Int. Cl. A61K 48/00; A61K 38/00; A61P 25/28; C07K 14/005; C07K 14/475; C12N 15/86

(52) U.S. Cl. CPC A61K 48/005; A61P 25/28; C07K 14/005; C07K 14/475; C12N 15/86; A61K 38/00; C07K 2319/00; C12N 2750/14122; C12N 2750/14143; C12N 2750/14145; C12N 2800/22

(57) ABSTRACT

The present disclosure generally relates to a recombinant adeno-associated viral (rAAV) vector comprising one or two of (a) a nucleotide sequence encoding Progranulin (PGRN), and (b) a nucleotide sequence encoding Stathmin-2 (STMN2), for treating proteinopathies in the central nervous system. Also disclosed are codon-optimized coding sequences of PGRN and/or STMN2, and expression cassettes, vectors, and viral particles comprising the same.

### CROSS-REFERENCE

This application is a continuation of Patent Cooperation Treaty application PCT/CN2024/095067, filed May 24, 2024 which claims the benefit of Patent Cooperation Treaty application PCT/CN2023/096582, filed May 26, 2023. Priority is claimed to these applications and the disclosures of these prior applications are considered part of the disclosure of this application and to the extent allowed the entire contents of the aforementioned applications are incorporated herein.

### REFERENCE TO SEQUENCE LISTING

In accordance with 37 CFR § 1.52(e)(5) and with 37 CFR § 1.831, the specification makes reference to a Sequence Listing submitted electronically as a .xml file named 196840138FPWOsequencelisting.xml. said .xml copy, created and filed in herewith is 119,000 bytes in size. The entire contents of the Sequence Listing are hereby incorporated by reference.

### FIELD OF THE INVENTION

The present disclosure relates to the technical field of gene therapy. Specifically, the present disclosure provides a recombinant adeno-associated viral (rAAV) vector comprising one or two of (a) a nucleotide sequence encoding Progranulin (PGRN), and (b) a nucleotide sequence encoding Stathmin-2 (STMN2), for treating neurodegenerative disorders, particularly amyotrophic lateral sclerosis (ALS), Frontotemporal Degeneration (FTD), Parkinson's disease (PD), multiple system atrophy (MSA), Alzheimer disease (AD), and other proteinopathies. Also provided herein are viral particles comprising the rAAV vector, a pharmaceutical composition comprising the viral particles, and uses thereof.

### BACKGROUND

Neurodegenerative disorders (NDs) affect millions of people which encompass a variety of conditions caused by selective dysfunction and progressive loss of cells in the brain or peripheral nervous system. Many NDs are also categorized as proteinopathies, since the aggregations formed by structurally abnormal protein are often observed in the dying nerve cells or their niche surrounding. These aggregations disrupt the normal function of neuronal cells resulting from either loss-of-function (LOF) or gain-of-function (GOF). The proteinopathies include amyotrophic lateral sclerosis (ALS), Frontotemporal Degeneration (FTD), Huntington's disease (HD), Parkinson's disease (PD), multiple system atrophy (MSA), Alzheimer disease (AD), and so on.

Amyotrophic lateral sclerosis (ALS) is a fatal proteinopathy. The motor neurons in ALS patient are progressively degenerated, which causes muscle weakness, loss of ambulation, and chronic disability in speaking and breathing leading to early death, 2-5 years after disease onset in most cases. In Europe, an estimated incidence of ALS is 1.75-3 per 100,000 persons per year and the prevalence is about 10-12/100,000. In mainland China, the prevalence of ALS was about 1.24/100,000 according to a recent report (Zhang J, Liu X, Liang H, Xu S, Wang X, Xu R. Amyotrophic lateral sclerosis in seven provinces of Chinese mainland: A cross-sectional survey from 2015 to 2016. Front Aging Neurosci. 2022 Sep. 15; 14:946353).

Currently, there is no really effective treatment for ALS, with the available treatments mainly focusing on symptom relieving and complication prevention. The Food and Drug Administration (FDA) has approved three small-molecule medicines for ALS, including Riluzole (Sanofi-Aventis), a free radical scavenger Edaravone (Radicava®; Mitsubishi Tanabe), and Relyvrio™ (Sodium phenylbutyrate and taurursodiol; Amylyx Pharmaceuticals). Riluzole could block glutamatergic neurotransmission and prolong the mean survival by 3-6 months. Both Edaravone and Relyvrio could slightly reduce the decline of daily functioning in ALS patients. Supporting therapies like physical therapy or breathing care are often used to alleviate the symptoms and assist patients' daily life. None the above treatments could reverse the disease progression.

The exact etiology of ALS is largely unknown. This disease is thought to be the consequence of complex interactions between genetic and environmental factors. About 10% of ALS are inherited in an autosomal dominant manner and defined as familial ALS (fALS), while the remaining ALS cases are sporadic (sALS) without any clear pattern such as family history. The genetic components in ALS are varied. In the fALS, genetic association studies have revealed that pathogenic variants frequently happened in several identified disease causative genes, like SOD1, C9ORF72, FUS, and TARDBP (Transactive response DNA-binding protein 43 kDa, TDP-43).

The neuropathological hallmark of ALS is the progressive death of both the upper motor neurons (UMNs) and lower motor neurons (LMNs), with ubiquitin-positive inclusions being detected in the dying cells. It was reported that TDP-43 is the main component of these inclusions. TDP-43 positive protein aggregations have been widely observed in ALS patients (>95%), indicating that the accumulation of TDP-43 aggregates could be the unifying component of ALS pathogenesis.

It is widely known that the TDP-43 pathology may contribute to neural degeneration in either a loss-of-function or gain-of-function manner. TDP-43 is a 43 kDa DNA-/RNA-binding protein encoded by the TARDBP gene. Physiologically, it plays important roles in gene transcription and translation, mRNA transport and stabilization, and so on. TDP-43 is a nuclear protein and the mutated TDP-43 will aggregate and lose its physiological location and thus its nuclear function. Through interactions with RNA transcripts of over 6,000 gene with critical function in axon formation, synaptic activity, and neuronal structure, TDP-43 plays an important role in regulating neuronal function. For example, in normal condition, TDP-43 binds and represses the inclusion of the cryptic exon (which harbors a stop codon) of Stathmin-2 (STMN2) gene, preventing it from aberrant splicing. In the absence of functional TDP-43, the protein levels of STMN2 are reduced, disrupting the microtubule stability in the axon growth cones.

The gain of toxic function (GOF) hypothesis of TDP-43 is supported by the observation that overexpressing either the wild-type or mutated TDP-43 consistently induced ALS-like neurodegenerative phenotypes in animal models as reported in many studies. Based on these findings, TDP-43 is a promising target for treating ALS. However, directly altering its protein or mRNA levels as a therapy might not be advantageous since the physiological level of TDP-43 is finely tuned.

Current investigational treatments for ALS targeting TDP-43 can be categorized into three types, clearance of TDP-43 aggregation by an intrinsic mechanism (e.g., PGRN pathway), targeting modifiers of TDP-43 mediated toxicity (e.g., Ataxin-2), or manipulation of the downstream genes of TDP-43 (e.g., STMN2).

Progranulin (PGRN) is a 593 amino acid secreted protein encoded by the GRN gene. The secreted PGRN appears to act extracellularly, through the tyrosine kinase ephrin type-A receptor 2 (EphA2) and the Notch signaling pathway, and plays a key role in neuronal survival and axonal outgrowth. PGRN also functions as a chaperone in the lysosomes to mediate the degradation of misfolded proteins to maintain the lysosomal homeostasis. PGRN is processed into multiple 6 kDa granulin (GRN) peptides in the lysosome.

The deficiency of PGRN has causal link with neuronal ceroid lipofuscinosis type 11 (CLN11), frontotemporal lobar degeneration with TDP-43 aggregation, and GRN type frontotemporal dementia (FTD-GRN), and is also linked to the progression of other neurodegenerative disorders, like ALS, PD, AD, and Autism.

There is a correlation between PGRN haploinsufficiency and TDP-43 aggregation probably through microglia mediated neuroinflammation. It was reported that overexpression of PGRN could reduce insoluble TDP-43 and rescue the ALS phenotype in the TDP-43 A315T transgenic mice (Beel, S., Herdewyn, S., Fazal, R. et al. Progranulin reduces insoluble TDP-43 levels, slows down axonal degeneration, and prolongs survival in mutant TDP-43 mice. Mol Neurodegeneration 13, 55 (2018)).

Prevail Therapeutics' PR006 is a single-dose gene therapy for treating FTD-GRN. PR006, a Phase 1/2 clinical trial drug is designed to deliver healthy PGRN protein into the central nervous system (CNS) of FTD patients by a rAAV vector.

WO2021058830A1 (filed by King's College London) discloses rAAV vectors comprising the codon optimized full-length human progranulin coding sequence. The rAAVs were evaluated for PGRN protein expression in mouse. Although WO2021058830A1 contemplated the use of the expression cassettes and rAAVs in treating frontotemporal dementia (FTD), neuronal ceroid lipofuscinosis (CLN11), amyotrophic lateral sclerosis (ALS), no disease model data was provided in the application.

As aforementioned, the mRNA splicing of STMN2 is directly regulated by the functional TDP-43. Antisense oligonucleotides (ASOs) which targets a cryptic exon site of STMN2 could mimic the function of TDP-43 by suppressing the cryptic splicing to restore the expression of the full-length STMN2, thus facilitating the axonal regeneration of human motor neurons (Michael W. Baughn et al., Mechanism of STMN2 cryptic splice-polyadenylation and its correction for TDP-43 proteinopathies. Science 379, 1140-1149 (2023)).

WO2021156832A1 discloses a miRNA inhibiting miR-485 for treating amyotrophic lateral sclerosis (ALS). It was generally recited in the application that the said miRNA could increase the expression of a series of genes whose down-regulation are thought to be associated with ALS, with one of the genes being STMN2. However, there's no data provided in the publication to support the effect of the said miRNA in regulating protein expression of STMN2 or its resulting effects on ALS disease progression.

WO2023018858A1 discloses a system for genetically editing Stathmin-2 (STMN2) based on CRISPR-Cas nuclease editing system, which can be used to treat ALS. The said system comprises a Cas12i polypeptide and a guide RNA mediating cleavage within the STMN2 gene. However, the said patent publication did not provide any experiments or data to support the therapeutic effect of the said system.

Frontotemporal dementia (FTD) is a clinically heterogeneous neurodegenerative disorder and probably the most common form of dementia in the younger population (45 to 65 age range). The mutations in three genes including C9ORF72, microtubule associated protein tau (MAPT) or GRN have causal links with the onset of FTD. According to the previous pathological studies, intracellular deposition containing abnormally aggregated proteins is often observed in the FTD brain tissues. The major components in the deposition include TDP-43, microtubule associated protein tau, and tumor-associated protein fused in sarcoma (FUS). Hence a TDP-43 targeting therapy could also be effective in treating the FTD patients.

Neuropathies often share similar pathological features. For instance, TDP-43 positive inclusions have been found not only in ALS and FTD, but also in other neuropathies, like primary lateral sclerosis, muscular atrophy, Guam Parkinson dementia complex, PD, a subpopulation of AD cases, inclusion body myopathy, and some traumatic brain injuries.

Current investigational therapies for proteinopathies usually target one aspect with a single mechanism of action (MOA), which may not be effective for treating these diseases with complicated etiology. Therefore, it remains an unmet medical need to develop more effective therapies for treating the multi-faceted proteinopathies.

### SUMMARY OF THE INVENTION

To develop more effective therapies for proteinopathies, the present inventors innovatively created a combination treatment targeting both LOF and GOF mechanisms of TDP-43 which could be a promising strategy and game changer to treat ALS, FTD, and other proteinopathies. In addition, the inventors have modified the nucleotide sequences encoding for PGRN and STMN2 to optimize their expression when delivered into the human brain via rAAV vectors. On such basis, rAAV vectors expressing the said two genes of interest (GOIs) in tandem are provided for use in therapies targeting multiple ALS pathologies synergistically so as to achieve greater therapeutic effects.

Therefore, in a first aspect, the present application provides an isolated nucleic acid molecule comprising a first polynucleotide sequence encoding a first polypeptide and a second polynucleotide sequence encoding a second polypeptide, wherein the first polypeptide is progranulin (PGRN) and the second polypeptide is stathmin-2 (STMN2); or the first polypeptide is stathmin-2 (STMN2) and the second polypeptide is progranulin (PGRN).

In one embodiment of the first aspect, the first polynucleotide sequence is located at 5′ upstream of the second nucleotide sequence.

In one embodiment of the first aspect, the progranulin (PGRN) comprises or is consisted of a polypeptide sequence of SEQ ID NO: 10, or a variant, homolog or orthohomolog thereof.

In one embodiment of the first aspect, the polynucleotide sequence encoding PGRN is a wild-type coding sequence, or a variant, homolog or orthohomolog thereof. In a preferred embodiment, the polynucleotide sequence encoding PGRN is a codon-optimized coding sequence. For example, the polynucleotide sequence encoding PGRN is codon-optimized for expression in human, for reducing CpG sites and/or for reducing CG contents. In specific embodiments, the polynucleotide sequence encoding for PGRN is a polynucleotide sequence selected from a group consisting of a polynucleotide sequence as shown in any one of SEQ ID NOs: 1-8.

In one embodiment of the first aspect, stathmin-2 (STMN2) comprises or is consisted of a polypeptide sequence of SEQ ID NO: 20, or a variant, homolog or orthohomolog thereof.

In one embodiment of the first aspect, the polynucleotide sequence encoding STMN2 is a wild-type coding sequence, or a variant, homolog or orthohomolog thereof. In a preferred embodiment, the polynucleotide sequence encoding STMN2 is a codon-optimized coding sequence. For example, the polynucleotide sequence encoding STMN2 is codon-optimized for expression in human, for reducing CpG sites and/or for reducing CG contents. In specific embodiments, the polynucleotide sequence encoding for STMN2 is a polynucleotide sequence selected from a group consisting of a polynucleotide sequence as shown in any one of SEQ ID NOs: 11-18.

In one embodiment of the first aspect, the first polynucleotide sequence and the second polynucleotide sequences are linked in frame and are operatively linked to a single promoter located at the 5′ upstream of both the first and the second nucleotide sequences, to form a combination construct. For example, the promoter is a constitutive promoter, e.g., an EF1α promoter or an EF1α-derived promoter. The EF1α-derived promoter can be a truncated version of EF1α promoter, such as an EFS promoter (a short version of the EF1α promoter). The EF1α-derived promoter can be a hybrid promoter consisting of the EF1α promoter or EFS promoter, and an additional nucleotide sequence, such as an intron sequence. In specific embodiments, the promoter can be selected from the hybrid promoters as disclosed in WO2023/061499, including EFShI1, EFShI2, EFShI3, EFSI4, EFSdI1 and EFSdI2, or variants thereof. In one specific embodiment, the promoter can be EFShI1 (SEQ ID NO: 27).

In one embodiment of the first aspect, the isolated nucleic acid molecule further comprises a linker sequence between the first and the second polynucleotide sequences. Preferably, the linker sequence is a coding sequence of a self-cleaving peptide or an internal ribosome entry site. Preferably, the self-cleaving peptide is a 2A peptide. For example, the 2A peptide is selected from a group consisting of E2A, F2A, T2A, and P2A, preferably P2A.

In one embodiment of the first aspect, the first polynucleotide sequence, the linker sequence and the second polynucleotide sequence are codon-optimized as a whole coding region. For example, the whole coding region is codon-optimized for expression in human. In specific embodiments, the isolated nucleic acid molecule comprises a codon-optimized coding region of both PGRN and STMN2 comprising or consisting of a polynucleotide sequence selected from a group consisting of SEQ ID NOs: 21-26. In specific embodiments, the isolated nucleic acid molecule comprises a codon-optimized coding region of both PGRN and STMN2 comprising or consisting of a polynucleotide sequence of SEQ ID NO: 22 or SEQ ID NO: 25, preferably SEQ ID NO: 25.

In one embodiment of the first aspect, the isolated nucleic acid molecule further comprises a polyadenylation signal. In specific embodiments, the polyadenylation signal is the SV40 polyA, or the human growth hormone (hGH) polyA.

In one embodiment of the first aspect, the isolated nucleic acid molecule further comprises a post-transcriptional regulatory element (WPRE) sequence.

In a second aspect, the present application provides a codon-optimized coding sequence of PGRN. Specifically, the coding sequence of PGRN is codon-optimized for expression in human, with reduced CpG sites and/or CG contents. In specific embodiments, the codon-optimized coding sequence of PGRN comprises or consists of a polynucleotide sequence as shown in any one of SEQ ID NOs: 1-8.

In a third aspect, the present application provides a codon-optimized coding sequence of STMN2. Specifically, the coding sequence of STMN2 is codon-optimized for expression in human, with reduced CpG sites and/or CG contents. In specific embodiments, the codon-optimized coding sequence of STMN2 comprises or consists of a polynucleotide sequence as shown in any one of SEQ ID NOs: 11-18.

In a fourth aspect, the present application provides an expression cassette comprising the isolated nucleic acid molecule of the first aspect, the codon-optimized coding sequence of PGRN of the second aspect, or the codon-optimized coding sequence of STMN2 of the third aspect. Preferably, the expression cassette is suitable for use in a recombinant adeno-associated viral (rAAV) vector.

In a fifth aspect, the present application provides a rAAV vector comprising the isolated nucleic acid molecule of the first aspect, the codon-optimized coding sequence of PGRN of the second aspect, the codon-optimized coding sequence of STMN2 of the third aspect, or the expression cassette of the fourth aspect.

In one embodiment of the fifth aspect, the AAV vector is an AAV vector of AAV9 serotype or proprietary ViVec serotypes.

In one embodiment of the fifth aspect, the rAAV vector further comprises two inverted terminal repeats (ITRs). In a preferred embodiment, the ITRs are AAV2 ITRs.

In a six aspect, the present application provides a viral particle comprising the rAAV vector of the fifth aspect.

In a seventh aspect, the present application provides a composition, e.g., a pharmaceutical composition, comprising the rAAV vector of the fifth aspect and a pharmaceutically acceptable excipient.

In an eighth aspect, the present application provides a method of treating or preventing neurodegenerative disorders (NDs) in a subject in need thereof, comprising administering the rAAV vector of the fifth aspect, the viral particle of the sixth aspect or the pharmaceutical composition of the seventh aspect to the subject. For example, the neurodegenerative disorders can be amyotrophic lateral sclerosis (ALS), Frontotemporal Degeneration (FTD), Huntington's disease (HD), Parkinson's disease (PD), multiple system atrophy (MSA), or Alzheimer disease (AD). In some embodiments, the neurodegenerative disorders can be neuropathies or proteinopathies, especially those associated with TDP-43 aggregation. For example, the neurodegenerative disorder can be TDP-43-associated ALS.

The bicistronic construct and rAAVs of the present application can simultaneously deliver and enable the expression of two therapeutic polypeptides, namely PGRN and STMN2. The inventive combined use of GRN and STMN2 as genes of interest in a gene therapy provides a novel strategy to develop a potentially more effective therapy in treating neurodegenerative disease via two synergistic mechanisms. By codon optimization of the coding sequences, both therapeutic polypeptides would be expressed at desirable levels, allowing for the intended use in treating specific neurodegenerative diseases. Moreover, the inventors surprisingly discovered that the rAAVs of the present application could achieve high levels of expression of both genes by the identified bicistroinic constructs of the present application, which has been found challenging in the art.

### BRIEF DESCRIPTION OF THE DRAWINGS

FIG. 1A and FIG. 1B show the schematics of the exemplary combination constructs containing both coding sequences of PGRN and STMN2 in different orders. FIG. 1A shows the schematic of PGRN-P2A-STMN2 combination construct and FIG. 1B shows the schematic of STMN2-P2A-PGRN combination construct.

FIG. 2 shows representative images of the Western blot showing the expression of PGRN protein in both cell lysate and supernatant samples and the expression of STMN2 in cell lysate samples. All samples were collected from N2A cells which were transfected with different combination constructs harboring codon optimized coding sequences of STMN2 and PGRN.

FIG. 3A shows the schematic of the exemplary construct containing a codon optimized coding sequence of PGRN. FIG. 3B shows the schematic of the exemplary constructs containing a codon optimized coding sequence of STMN2.

FIG. 4 shows representative images of the Western blot results of GRN protein expression in both cell lysate and supernatant samples collected from SH-SY5Y cells which were transfected with different constructs comprising codon optimized coding sequence of GRN.

FIG. 5 shows the representative images of the Western blot results of STMN2 protein expression in the cell lysate samples collected from SH-SY5Y cells which were transfected with different constructs comprising codon optimized coding sequence of STMN2.

FIG. 6 presents Western blot results of GRN and STMN2 protein expression in N2A-AAVR cells transduced with one of the combination constructs AAV9-MC2 and AAV9-MC5. GRN protein levels in both the cytosolic (cell lysate) and extracellular (supernatant) fractions were detected, while only the cytosolic STMN2 protein levels were detected as the protein is localized exclusively in the cytosol.

FIG. 7 depicts the survival curve of the male ALS mice following intrathecal administration of AAV9-MC5.

FIG. 8 shows the latency (seconds; sec) in the Rotarod test for male ALS mice at 8 weeks following the intrathecal administration of AAV9-MC5.

FIG. 9 presents the statistical analysis of the Western blot results for insoluble TDP-43 levels in the cortex and spinal cord tissue samples collected from ALS mice at 15 weeks post-intrathecal administration of AAV9-MC5. L: Low dose; H: High dose.

FIG. 10 presents a statistical analysis of immunostaining results for TDP-43 levels in fixed spinal cord tissue samples from ALS mice at 15 weeks post-intrathecal administration of AAV9-MC5. TDP-43 levels are quantified by both the number of TDP-43 positive cells and the total area occupied by TDP-43 positive staining. L: Low dose; H: High dose.

FIG. 11 shows representative images and a statistical analysis of the number of TDP-43 positive aggregates in the ALS cell model following transduction with AAV9-MC5 (MOI=1E+6). White arrows indicate the TDP-43 positive aggregates.

### DETAILED DESCRIPTION OF THE INVENTION

Unless specifically defined elsewhere in this document, all of the technical and scientific terms used herein have the meaning commonly understood by one of ordinary skill in the art to which this invention belongs.

As used herein, including the appended claims, the singular forms of words such as “a”, “an”, and “the”, include their corresponding plural references unless the context clearly dictates otherwise.

In the context of the present disclosure, unless being otherwise indicated, the wording “comprise”, and variations thereof such as “comprises” and “comprising” will be understood to imply the inclusion of a stated element, e.g. an amino acid sequence, a nucleotide sequence, a property, a step or a group thereof, but not the exclusion of any other elements, e.g. amino acid sequences, nucleotide sequences, properties and steps. When used herein the term “comprise” or any variation thereof can be substituted with the term “contain”, “include” or sometimes “have” or equivalent variation thereof. In certain embodiments, the wording “comprise” also include the scenario of “consisting of”.

The term “gene” as used herein refers to a nucleic acid (such as DNA, e.g., genomic DNA or cDNA) and its corresponding nucleotide sequence encoding an RNA transcript. As used herein, terms with reference to genomic DNA can include intervening non-coding regions as well as regulatory regions, and may include both 5′ and 3′ terminus. In some instances, the term includes transcribed sequences, including 5′ and 3′ untranslated regions (5′-UTR and 3′-UTR), exons and introns. In some genes, the transcribed regions will contain an “open reading frame” encoding the polypeptide. In some instances, a “gene” comprises only the coding sequence (e.g., an “open reading frame” or “coding region”) necessary to encode a polypeptide. In some instances, the term “gene” includes not only transcribed sequences, but also non-transcribed regions, including upstream and downstream regulatory regions, enhancers, and promoters. A gene may refer to an “endogenous gene” or a native gene. A gene may refer to a “foreign gene” or a non-native gene. A non-native gene can refer to a gene not normally found in the host organism but introduced into the host organism by gene transfer. A non-native gene can also refer to a gene that is not in its natural location in the genome of an organism. A non-native gene can also refer to a naturally occurring nucleic acid that contains mutations, insertions and/or deletions (e.g., non-native sequences), e.g., a codon-optimized nucleotide sequence. In the context of the present application, by “GOI” it specifically refers to CDS region, namely the sequences coding for amino acids in a protein, unless being otherwise indicated.

The terms “polynucleotide”, “oligonucleotide” and “nucleic acid” are used interchangeably herein and refer to a polymeric form of nucleotides of any length. A polynucleotide can be exogenous or endogenous to a cell. A polynucleotide can exist in a cell-free environment. A polynucleotide can be a gene or a fragment thereof. A polynucleotide can be DNA. A polynucleotide can be RNA. A polynucleotide can have any three-dimensional structure and can perform any function, known or unknown. A polynucleotide may contain one or more analogs (e.g., altered backbones, sugars, or nucleobases).

The term “isolated nucleic acid” means a DNA or RNA which is removed from all or a portion of a polynucleotide in which the isolated polynucleotide is found in nature, or is linked to a polynucleotide to which it is not linked in nature. An isolated nucleic acid molecule “comprising” a specific nucleotide sequence may include, in addition to the specified sequence, operably linked regulatory sequences that control expression of the coding region of the recited nucleic acid sequences. Due to the codon degeneracy, one skilled in the art understands that any specific amino acid sequence can be coded by several different nucleotide sequences.

The term “expression cassette” herein refers to a DNA component included in a vector (e.g., rAAV vector) and consisted of one or more, for example one or two GOIs selected from GRN and STMN2 genes under the control of regulatory sequences to be expressed in a host cell transduced by the rAAV vector.

The term “combination construct” in the context of the present application refers to a construct comprising two GOIs, specifically GRN and STMN2. In preferred embodiments, the combination construct is a bicistronic construct, in which the two genes of interest can be transcribed in a single mRNA. For example, the two coding sequences of PGRN and STMN2 are constructed in frame under the control of the same promoter located at 5′ upstream of both coding sequences.

“Operatively linked” as described herein is used to describe that two or more components, particularly nucleotide sequences, are connected in a way that each of the components can perform their designated functions.

“Codon-optimized coding sequence” herein refers to a nucleotide sequence coding for a protein, such as PGRN or STMN2, modified from their wild-type coding sequence accommodating codon bias.

“AAV” refers to adeno-associated virus. “rAAV” refers to recombinant adeno-associated virus.

“PGRN” refers to the protein progranulin, which is encoded by the gene GRN in human. “GRN” refers to granulin, which is a group of secreted peptides derived from progranulin by cleavage in the lysosome.

“STMN2” refers to Stathmin-2. STMN2 can be interchangeably referred to as “SCG10”.

“CpG island” refers to a region in the genome rich in CpG sites. “CpG site” refers to two consecutive nucleotides consisting of a cytosine (C) and a guanine (G) in a 5′ to 3′ direction.

“2A peptide” refers to a group of short (18-22 amino acids) self-cleaving peptides derived from viruses. 2A peptides create ribosome skipping during translation, leading to separation between the end of the 2A sequence and the downstream protein.

“Proteinopathy” refers to a neurodegenerative disorder with accumulation of structurally abnormal protein, for example, TDP-43, leading to the formation of aggregates or inclusions in axons of neurons or oligodendrocytes.

In the context of the present application, “subject” refers to an animal, preferably a mammal, such as a rodent, e.g., a mouse or rat, or a primate, e.g., a cynomolgus monkey, preferably a higher primate, such as a human. Unless otherwise stated, the term “subject” is interchangeable with the term “patient” or “individual” in the context of this application.

### Expression Cassette

In one embodiment, the expression cassette of the present application is characterized by expression of one GOI, e.g., a coding sequence of either PGRN or STMN2, preferably a codon-optimized coding sequence of either PGRN or STMN2, in particular those as recited in the present disclosure. For example, the codon-optimized coding sequence of PGRN can be selected from a polynucleotide sequence of any one of SEQ ID NO: 1-8, or a polynucleotide having at least 85%, at least 90%, at least 95%, at least 97%, at least 98%, at least 99% sequence identity of any one of SEQ ID NO: 1-8. For example, the codon-optimized coding sequence of STMN2 can be selected from a polynucleotide sequence of any one of SEQ ID NOs: 11-18, or a polynucleotide having at least 85%, at least 90%, at least 95%, at least 97%, at least 98%, at least 99% sequence identity of any one of SEQ ID NOs: 11-18.

Codon optimization may be achieved by reducing sequence complexity, adjusting GC content, adjusting codon usage and/or avoiding rare codons. The coding sequence which has been codon optimized usually shows an increased translational efficiency of the gene of interest (GOI), leading to a higher protein expression. Tools (e.g., JCat) with embedded algorithm to design codon optimized coding sequence are readily accessible to those skilled in the art.

In a preferred embodiment, the codon of the PGRN and/or STMN2 coding sequence of the present application has a Codon Adaptation Index (CAI) of at least 0.75, preferably of at least 0.8, more preferably of at least 0.85. CAI is a measure of codon bias. One skilled in the art would understand that the actual efficiency of any sequence generated by running an algorithm still needs to be verified by experiments.

In a preferred embodiment, the PGRN and/or STMN2 coding sequence of the present application has a reduced number of CpG sites or no CpG site as compared to corresponding wild type coding sequence. In a preferred embodiment, the PGRN and/or STMN2 coding sequence of the present application has a reduced level of CG content, e.g., a CG content of no more than 60%.

By codon optimization, the expression cassette of the present disclosure after being inserted into an AAV vector can achieve higher and more consistent protein expression or co-expression in neuronal cells in vitro or in vivo. For example, the expression cassette of the present disclosure shows better performance in expression of GOI(s) in human cell lines with neuronal identity, such as SH-SY5Y cells. Since progranulin is a secretory protein, its expression can be evaluated by measuring the levels of protein in the supernatant of the cell culture, and the total protein amount in both supernatant and lysate of the cell culture.

In preferred embodiments, the expression cassette of the present application is a bicistronic expression cassette characterized by co-expression of PGRN- and STMN2-coding sequences spaced by a linker sequence. Either of the coding sequences can be wild type coding sequence or a codon-optimized coding sequence. For example, the coding sequence of PGRN in bicistronic expression cassette can be selected from a polynucleotide sequence of any one of SEQ ID NOs: 1-9, or a polynucleotide having at least 85%, at least 90%, at least 95%, at least 97%, at least 98%, at least 99% sequence identity of any one of SEQ ID NOs: 1-9. For example, the coding sequence of STMN2 in bicistronic expression cassette can be selected from a polynucleotide sequence of any one of SEQ ID NOs: 11-19, or a polynucleotide having at least 85%, at least 90%, at least 95%, at least 97%, at least 98%, at least 99% sequence identity of any one of SEQ ID NOs: 11-19.

In one specific embodiment, the expression cassette of the present disclosure comprises a polynucleotide sequence encoding PGRN and a polynucleotide sequence encoding STMN2, wherein the polynucleotide sequence encoding PGRN is SEQ ID NO: 1 or SEQ ID NO: 4. In another specific embodiment, the expression cassette of the present disclosure comprises a polynucleotide sequence encoding PGRN and a polynucleotide sequence encoding STMN2, wherein the polynucleotide sequence encoding STMN2 is SEQ ID NO: 13 or SEQ ID NO: 14.

The coding sequence of PGRN and STMN2 can be arranged in either order in the combination construct of the present application, since the present inventors discovered that changing the order had limited influence on the relative expression and/or transduction levels of the two GOIs.

The linker sequence of the present application can produce high efficiency and fidelity when the linker sequence is used to connect two coding sequences to be co-expressed in a single rAAV vector. As an example of the linker sequence, a sequence coding for 2A peptide (such as P2A, F2A, or E2A) can be used to connect the two coding sequences of the present application. The position of GOI(s) relative to the linker sequence can be adjusted to achieve desirable protein expression and function. In preferred embodiments, a P2A linker sequence is used in the rAAV between the two GOIs. In specific embodiments, the linker sequence of the present application comprises or consists of a nucleotide sequence as shown in any one of SEQ ID NO: 31, SEQ ID NO: 32 and SEQ ID NO: 33.

The codon optimization can also be conducted on the whole coding region comprising both genes of interest as well as the linker sequence. For example, the coding region comprises a codon-optimized polynucleotide sequence selected from any one of SEQ ID NOs: 21-26, or a polynucleotide having at least 85%, at least 90%, at least 95%, at least 97%, at least 98%, at least 99% sequence identity of any one of SEQ ID NOs: 21-26. In preferred embodiments, the coding region comprises a codon-optimized polynucleotide sequence selected from any one of SEQ ID NO: 22 (MC2) or SEQ ID NO: 25 (MC5). In more preferred embodiments, the coding region comprises a codon-optimized polynucleotide sequence selected from SEQ ID NO: 25 (MC5).

Aside from the coding sequences or coding regions, the expression cassette can further comprise one or more regulatory sequences. “Regulatory sequence” in the context of the present application refers to a nucleotide element that has influence on the expression of the genes of interest. Regulatory sequence can be selected from one or more of promoter, enhancer, polyadenylation sequence, and translation termination signal. A certain combination of regulatory sequences of the present disclosure can achieve unexpected effect in improving the expression efficiency of the coding sequence.

“Promoter” refers to a DNA sequence enables initiation of transcription of a downstream gene under the control of the said promoter. Promoters include but not limited to constitutive promoters, cell type-specific promoters, tissue-specific promoters, and development stage-specific promoters. The tissue-specific promoter can be a brain-specific promoter. Promoter can be a naturally occurring promoter of a gene, a modified version of a naturally occurring promoter or a synthetic promoter.

In the preferred embodiments, the promoter of the present disclosure can be a constitutive promoter. In the preferred embodiments, the promoter can be a EF1α-derived promoter (e.g., EFS-derived promoter, such as an EFShI1 promoter), a CBh promoter, an EF1α promoter, a CAG promoter, an MBP promoter (myelin basic protein promoter) or a promoter derived therefrom. In specific embodiments, the promoter is an EFShI1 promoter having a nucleotide sequence of SEQ ID NO: 27.

“Enhancer” is a regulatory DNA sequence which can enhance the transcription of the GOI in rAAV together with the promoter. In a preferred embodiment, the expression cassette of the present application comprises of an enhancer. More preferably, the enhancer can be a CMV enhancer, e.g., in a CBh promoter.

In some embodiments, intron sequences functioning as enhancers can be included. For example, an intron sequence originated from the intron or untranslated region (UTR) of a respective GOI can be included in the expression cassette.

In some cases, a promoter together with an enhancer and/or an intron sequence are collectively referred to as “promoter” or “promoter element”. In a preferred embodiment, the promoter is the CBh promoter. In another preferred embodiment, the promoter is consisted of the EFS promoter and an intron sequence.

Preferably, the intron sequence has a total length of about or less than 200 bp, about or less than 250 bp, about or less than 300 bp, about or less than 350 bp, about or less than 400 bp.

For example, the intron sequence of the present disclosure is derived from the gene of interest. For example, the intron sequence is consisted of one or more fragments derived from one or more intronic regions of the gene of interest.

In a preferred embodiment, the promoter or promoter/intron element has a length of no more than 1000 bp, no more than 900 bp, no more than 850 bp, no more than 800 bp, no more than 700 bp, no more than 600 bp, no more than 500 bp, or no more than 400 bp, due to the limited packaging capacity of AAV.

In some cases, when the intron sequence is derived from an intronic region of the gene of interest, it can be inserted into the coding sequence (e.g., codon-optimized coding sequence) at a position corresponding to the position where it locates in the gene in nature, e.g., between two exons, instead of locating at a position 5′-upstream of the coding sequence and constituting a promoter/intron element.

The Kozak consensus sequence (Kozak sequence), named after the scientist who discovered it, is a nucleic acid sequence motif present in most eukaryotic mRNA transcripts that functions as the protein translation initiation site. Kozak sequence ensures the protein is accurately and efficiently translated.

The expression cassette of the present application can contain a polyadenylation signal (Poly A). For example, the Poly A sequence can be used in the present application includes SV40 polyA, human growth hormone (hGH) polyA, or bovine growth hormone (bGH) polyA.

In one embodiment, the expression cassette of the present disclosure comprises a SV40 polyA having polynucleotide sequence of SEQ ID NO: 28. In one embodiment, the expression cassette of the present disclosure comprises a hGH poly A having polynucleotide sequence of SEQ ID NO: 29.

In some embodiments, the expression cassette of the present disclosure comprises a woodchuck hepatitis virus post-transcriptional regulatory element (WPRE). WPRE sequence can be placed downstream of the GOI and proximal to the polyadenylation signal.

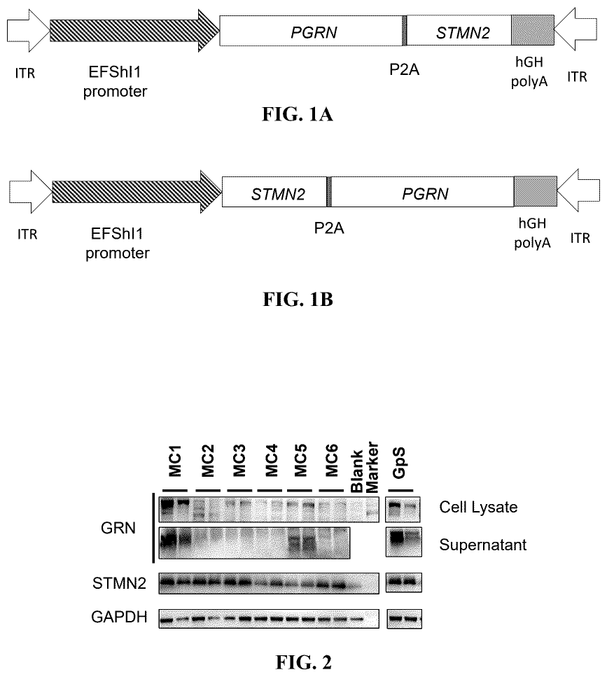

In a specific embodiment, the expression cassette of the present disclosure comprises an EFShI1 promoter, a coding sequence of PGRN, a linker sequence encoding P2A peptide, a coding sequence of STMN2, and a hGH polyA sequence; or an EFShI1 promoter, a coding sequence of STMN2, a linker sequence encoding P2A peptide, a coding sequence of PGRN, and a hGH polyA sequence, as shown in FIG. 1A and FIG. 1B.

### AAV Vectors

The expression cassette of the present application is suitable for use in rAAV vectors. Accordingly, the present application provides rAAV vectors comprising the coding sequences, or the expression cassette of the present application. Unless being otherwise indicated, the term “rAAV vector” in the context of the present application refers to the rAAV vector plasmid.

The rAAV vector of the present application comprises a heterologous polynucleotide, e.g., the expression cassette of the present application, flanked by two ITRs. The ITRs can be of any suitable serotype. Preferably, both ITRs are AAV2 ITRs or variants thereof. The nucleotide sequence of the AAV2 ITR is known in the art.

The rAAV vector can be a single stranded AAV (ssAAV) or a self-complementary AAV (scAAV).

Furthermore, the rAAV viral particle of the present application comprises AAV capsid protein and the rAAV vector encapsulated in said capsid protein. In some embodiments, the rAAV of the present application comprises a modified or engineered AAV capsid as compared to the wild-type AAV capsid.

The present application contemplates the use of AAV vector of any serotype. However, the rAAV vector of a serotype showing tissue tropism of CNS is preferred. For example, the rAAV vector of the present application is preferably AAV9 vector or a ViVec AAV vector.

Vivec AAV vectors are a series of vectors comprising an engineered adeno-associated viral (AAV) capsid polypeptide and showing an improved CNS tropism as compared to the wild-type capsid polypeptide of AAV9. The capsid polypeptide of a ViVec AAV vector comprises an insertion of 7 amino acids at a position between the amino acid position Q588 and position A589 of the wild-type AAV9 VP1 capsid polypeptide having an amino acid sequence as shown SEQ ID NO: 34. In specific embodiments, the 7-amino acid insertion sequence of a ViVec AAV vector is selected from a group of amino acid sequences as shown in SEQ ID NOs: 35-94 which give rise to AAV vectors designated as ViVec-N001 to ViVec-N060, respectively. In preferred embodiments, the 7-amino acid insertion sequence of a ViVec AAV vector is selected from a group of amino acid sequences as shown in SEQ ID NO: 36, SEQ ID NO: 38, SEQ ID NO: 39, SEQ ID NO: 40, SEQ ID NO: 41, SEQ ID NO: 42, SEQ ID NO: 55, SEQ ID NO: 56, SEQ ID NO: 57, SEQ ID NO: 58, SEQ ID NO: 60, SEQ ID NO: 63, SEQ ID NO: 64, SEQ ID NO: 67, SEQ ID NO: 74 and SEQ ID NO: 77, which correspond to ViVec-N002, ViVec-N004, ViVec-N005, ViVec-N006, ViVec-N007, ViVec-N008, ViVec-N021, ViVec-N022, ViVec-N023, ViVec-N024, ViVec-N026, ViVec-N029, ViVec-N030, ViVec-N033, ViVec-N040, and ViVec-N043, respectively.

### Pharmaceutical Composition

The term “pharmaceutical composition” refers to a composition suitable for delivering to a subject. The pharmaceutical composition of the present disclosure comprises the isolated nucleic acid, the rAAV vector or the viral particle of the present disclosure and a pharmaceutically acceptable excipient. Conventional pharmaceutically acceptable excipients are known in the art and can be solid or liquid excipients. In one embodiment, the pharmaceutical composition can be a liquid for injection.

### Delivery Methods

The terms “administration”, “administering”, “treating” and “treatment” as used herein, when applied to a subject, e.g., an animal, including human, or to cell, tissue, organ, or biological fluid, means contact of an exogenous pharmaceutical, therapeutic, diagnostic agent, or composition with the subject, cell, tissue, organ, or biological fluid. Treatment of a cell encompasses contact of a reagent with the cell, as well as contact of a reagent with a fluid, where the fluid is in contact with the cell. The term “administration” and “treatment” also include in vitro and ex vivo treatments, e.g., of a cell, by a reagent, diagnostic, binding compound, or by another cell.

In preferred embodiments, the rAAV vector of the present application can be delivered via intravenous, intra cerebroventricular, intrathecal or intra-striatum administration. In a specific embodiment, the rAAV vector is delivered via intrathecal route.

The rAAV vector can be administered via a single dose or multiple doses. In a specific embodiment, the rAAV vector is administered via a single injection.

The dosage of the rAAV vector injection can be varied based on the administration route. The dosage can also be varied based on the body weight of the subject. Therefore, the dose range can be within a broad scope which covers 1.5×10⁹-1.5×10¹⁴vg/kg.

### Therapeutic Uses

The term “treat”, “treating” or “treatment” includes to cure or at least to alleviate the symptoms of a neurodegenerative disorder, such as amyotrophic lateral sclerosis (ALS), Frontotemporal Degeneration (FTD), Huntington's disease (HD), Parkinson's disease (PD), multiple system atrophy (MSA), or Alzheimer disease (AD) or other proteinopathies, especially those associated with TDP-43 abnormal aggregation. By “associated with”, it means that the neurodegenerative disorder is accompanied by the TDP-43 abnormal aggregation, while it does not necessarily mean that the neurodegenerative disorder is caused by TDP-43 abnormal aggregation.

A subject having any of these neurodegenerative disorders can be diagnosed by a well-trained neurologist based on the genetic background, medical history, symptoms and signs, as well as the results of neurological and physical examinations, according to the Clinical Diagnostic Criteria.

In specific embodiments, the subject can be a clinically diagnosed ALS patient. ALS to be treated by the rAAV vector of the present application may suffer from familial ALS (fALS) or sporadic (sALS).

Alleviation of the neurodegenerative disorder, e.g., ALS, by administering the rAAV vector of the present application can be shown as reduced progression of motor neuron death, reduced number of ubiquitin-positive inclusions, and/or reduced accumulation of TDP-43 aggregates.

In the treatment of aforesaid NDs, the rAAV vectors of the present application comprising a combination construct, e.g., the bicistronic rAAV vectors can be used. Alternatively, two rAAV vectors, each harboring one of the PGRN coding sequence and STMN2 coding sequence of the present application, can be used. The bicistronic rAAV vectors are preferred.

### EXAMPLES

To facilitate the understanding and utilization of the present invention, the merits of the present invention will be described in more details with reference to examples and appended drawings. However, it should be understood that the following examples only intend to exemplify the present invention without any intention in limiting the scope of the present invention. The scope of the present invention should be defined by the claims.

### Example 1. Codon-Optimized Single GOI Constructs and Combination Constructs Generated by Conducting Codon Optimization on Combination Constructs

To generate combination constructs to express both PGRN and STMN2 protein by rAAV, a codon optimization procedure was conducted to improve protein expression. The wild type coding sequences of GRN (G0; SEQ ID NO: 9) and STMN2 (S0; SEQ ID NO: 19) were linked by P2A sequence (P2A-3; SEQ ID NO: 33) to generate G0S0 (GpS) and S0G0 (SpG). In G0S0, the progranulin coding sequence is located upstream of the Stathmin-2 coding sequence, while in S0G0, the order of the two GOIs was reversed. G0S0 and S0G0 were used as reference sequences.

Then, based on the reference G0S0 and S0G0 sequence, six manually optimized combination sequence MC1-MC6 (SEQ ID NOs: 21-26) were generated by subjecting the entire coding region including the coding sequences of both progranulin and Stathmin-2 linked by an intervening P2A sequence to codon optimization. Higher frequency codons (human specific) were used to replace the wild-type codons in the construct, while leaving the polypeptide sequence unchanged. The sequence forming hairpin and repeated sequences were avoided during optimization. These optimized sequences have Codon Adaptation Index (CAI) greater than 0.75 as calculated by an online tool (https://www.genscript.com/tools/rare-codon-analysis), while harbor reduced CG contents.

MC1, MC2, MC3, and MC6 are four different versions of the codon optimized constructs. In MC1 and MC3, the progranulin coding sequence was placed before the Stathmin-2 coding sequence, while in MC2 and MC6, the order of the two GOIs was reversed. MC4 and MC5 were generated by reversing the order of GOIs while keeping their sequences in M3 and M6, respectively.

The eight different combination sequences (MC1-MC6, GpS, SpG) were then inserted into a AAV vector backbone plasmid, consisted of the following elements in sequence: EFShI1 promoter (SEQ ID NO: 27), the first coding sequence, P2A sequence (SEQ ID NO: 31, SEQ ID NO: 32, or SEQ ID NO: 33), the second coding sequence, hGH polyA (SEQ ID NO: 29) as shown in FIG. 1A and FIG. 1B, as well as two AAV2 ITRs. The detailed information of MC1-MC6 is shown in Table 1.

TABLE 1
The combination construct information of MC1-MC6

| Construct | 1^{st} coding sequence | P2A | 2^{nd} coding sequence |
|---|---|---|---|
| MC1 | G1 | P2A-1 | S1 |
| MC2 | S2 | P2A-2 | G2 |
| MC3 | G3 | P2A-3 | S3 |
| MC4 | S3 | P2A-3 | G3 |
| MC5 | G4 | P2A-3 | S4 |
| MC6 | S4 | P2A-3 | G4 |

These six MCs and GpS were transfected into N2A cells (a mouse neuroblastoma cell line, Procell #CL-0168) with jetOPTIMUS® (Polyplus-Transfection®, #101000025) following the instruction manual provided by the manufacturer. 72 hours after the transfection, supernatant in the culture well was collected and concentrated to 200 μL (1:10 ratio) by 30 kDa centrifugal devices (Amicon® Ultra-4 Centrifugal Filter Unit, Millipore, UFC8030) to measure the amounts of the secreted progranulin protein.

Cell lysates were also collected to measure the protein levels of progranulin and Stathmin-2. The cells in the culture well were washed once with 1×PBS and harvested in 150 μL RIPA buffer (Beyotime, P0013B or Thermo Scientific™, #89900) containing protease inhibitor cocktail (Roche, 11697498001) to generate cell lysates. The cell lysate samples were then collected after sonication and centrifugation.

### BCA Assay

The protein concentration of progranulin in both the supernatant and cell lysate samples were determined by BCA assay (Beyotime, P0010S). All of the samples were boiled at 95° C. for 10 min after adding the reducing loading buffer.

### Western Blot

The protein levels of progranulin in both the cell lysate and supernatant were also determined by western blot (WB). All samples (30 μg loading amount) were separated on the SDS polyacrylamide gels (GenScript, M00657) along with the pre-stained protein ladder (Thermo Scientific, 26616) and transferred onto a Nitrocellulose membrane (Pall, 55253088) for 2 hours at 80V. The membrane was washed in TBST (Solarbio, T1081) and then blocked by 5% BSA blocking buffer (Solarbio, SW3015) at 4° C. overnight. The membrane was incubated with an anti-PGRN antibody (R&D, #AF2420 or Abclonal. #A5124 or Sino biological, #10826-R007), or an antibody against the housekeeping gene β-actin (Sigma, #A1978) overnight at 4° C., followed by 2 hour incubation with a Dye-conjugated secondary antibody at room temperature overnight. The signals were then detected by ODYSSEY Clx infrared imaging system (LI-COR Biosciences).

The protein levels of Stathmin-2 in cell lysate samples were determined by WB using a primary antibody against Stathmin-2 (Invitrogen, #PA5-21768, Proteintech, #10586-1-AP or Abcam, #ab185956).

### Sandwich ELISA

The protein levels of progranulin in both cell lysate and supernatant were also determined by a sandwich ELISA (Sino Biological, KIT10826) according to the manual provided by the manufacturer.

### Results

The progranulin levels in both supernatant and cell lysate and the Stathmin-2 levels in cell lysate were determined by WB. As shown in FIG. 2, MC1 and MC5 expressed more progranulin in both the supernatant and cell lysate among the six candidates and MC1 outperformed GpS.

MC1-MC3 expressed similarly high levels of Stathmin-2. Surprisingly, the expression level of STMN2 in MC3 was higher than that in MC4, indicating no drop-off (expression reduction) for STMN2 in the downstream position, as normally observed for combination constructs in which the two GOIs are prepared into a single expression cassette driven by a single promoter.

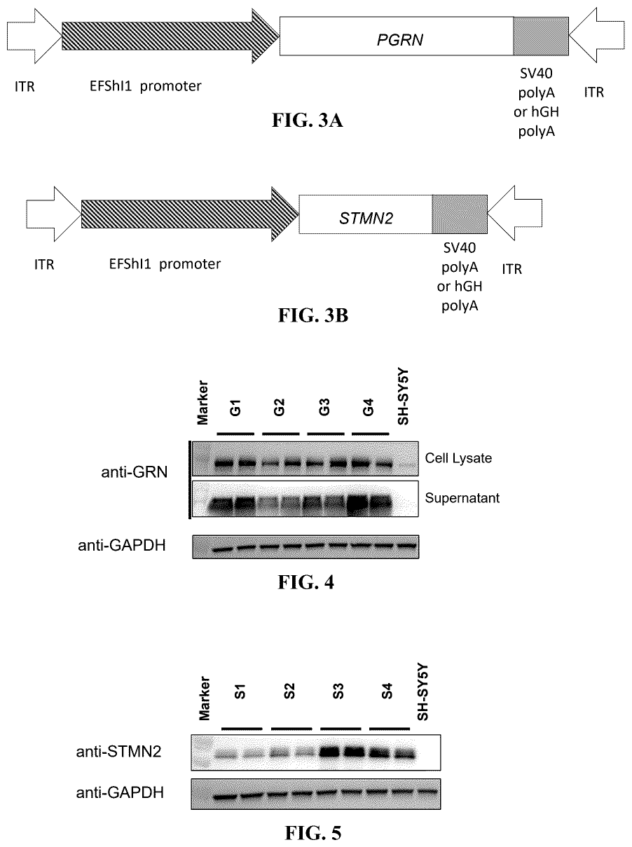

Constructs harboring only GRN or STMN2 were also generated to use as controls to evaluate the combination effects in in vitro functional assays and in vivo studies. The codon optimized sequences of GRN (G1-G4) or STMN2 (S1-S4) were extracted from MC1-MC6 and inserted into the AAV vector backbone plasmid, consisted of the following elements in sequence: an EFShI1 promoter, G or S coding sequence, and a hGH polyA sequence, flanked by two AAV2 ITR (FIG. 3A and FIG. 3B).

These “one-GOI” constructs derived from MCs were also transfected into SH-SY5Y cells (a human neuroblastoma cell line, purchased from Procell, #CL-0208) using Lipofectamine 3000 transfection reagent (Invitrogen, #L3000150) following the manufacturer's instructions.

The cell lysate and supernatant samples were collected as mentioned above to measure the protein levels of progranulin or Stathmin-2 by WB. As shown in FIGS. 4 and 5, among the tested candidates, G1 and G4 showed higher protein expression level of PGRN (FIG. 4), while S3 and S4 showed higher protein expression level of STMN2 (FIG. 5). Interestingly, G1 derived from MC1 and G4 derived from MC5 maintained their expression performances as observed in the combination constructs, while for STMN2 expression, only S3 from MC3 and S4 from MC6 expressed high levels of STMN2, but not S1 and S2 derived from MC1 and MC2.

### Example 2. Codon-Optimized Single GOI Constructs and Combination Constructs Generated by Conducting Independent Codon Optimization on Each GOI

Codon optimization of the individual GOI was also conducted. Four additional coding sequences containing higher frequency codons were generated for GRN (named G5-G8, SEQ ID NOs: 5-8) and STMN2 (named S5-S8, SEQ ID NOs: 15-18). All of these sequences have Codon Adaptation Index (CAI) greater than 0.85 as calculated by an online tool (https://www.genscript.com/tools/rare-codon-analysis), while harbor no CpG island and with reduced CG contents. The wild-type coding sequences of GRN (G0, SEQ ID NO: 9) and STMN2 S0, SEQ ID NO: 19) were also synthesized as references.

Ten candidate constructs with configurations as shown in FIG. 3A and FIG. 3B were generated with aforesaid eight codon-optimized sequences and the two wild-type reference sequences. Specifically, each of the synthesized PGRN or STMN2 coding sequence was cloned into a vector backbone which harbors the EFShI1 promoter (SEQ ID NO: 27) and a SV40 polyA tail (SEQ ID NO: 28) or a hGH polyA tail hGH polyA (SEQ ID NO: 29), as well as two AAV2 ITRs.

Besides MC1-MC6, a second batch of combination constructs containing all thirty-two possible combinations of the individually codon optimized sequences of the two GOIs in an order of either G+S or S+G (G5S5, G5S6, G5S7, G5S8, G6S5, G6S6, G6S7, G658, G755, G7S6, G7S7, G758, G855, G8S6, G8S7, G8S8, G1S3, G1S4, and G4S3; and the aforesaid constructs in reverse order) were generated and the protein expression of both GOIs were evaluated as described for the first batch of the combination constructs in Example 1.

### Example 3. Evaluation of Protein Expression of the rAAV9 Vectors Comprising the Combination GOI Constructs and the Single GOI Constructs

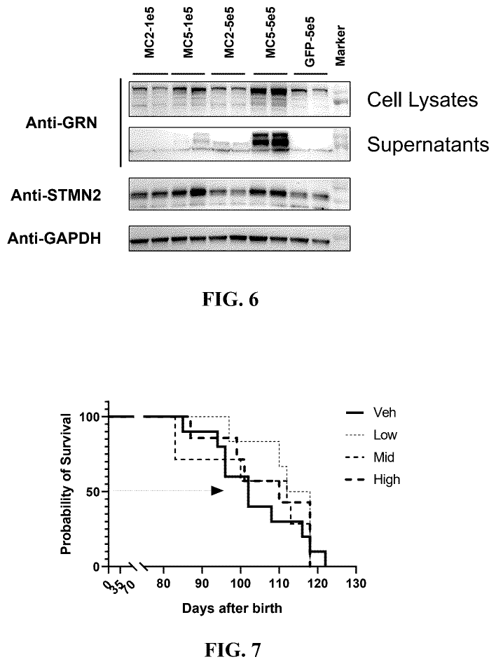

Combination GOI construct plasmids as described in Example 1 were prepared and packaged into AAV9. The rAAVs were diluted in the culture medium and added to N2A-AAVR cells (an in-house developed N2A cell line overexpressing the AAV receptor) at two different MOIs (1E+5 and 5E+5) to transduce the cells. The cells were harvested 72 hours post transfection as described earlier to evaluate protein expression by WB. As shown in FIG. 6, AAV9-MC5 (G4S4) expressed higher levels of the GRN and STMN2 proteins than AAV9-MC2 (S2G2).

### Example 4. In Vivo Therapeutic Efficacy of the rAAV Candidates Expressing the Optimized GRN and STMN2 in a hTDP-43 A315T Transgene ALS Mouse Model

The therapeutic effects of the rAAVs comprising the selected combination GOI construct MC5 were evaluated in an ALS mouse model (hTDP-43 A315T ALS mouse, a mouse model overexpressing the human TDP-43 A315T transgene to recapitulate the mutant TDP-43 induced ALS phenotype).

AAV9-MC5 was injected into the hTDP-43 A315T ALS mice via intrathecal injection at three different doses (Low: 3.00E+8 vg, Mid: 1.00E+9 vg, High: 3.00E+9 vg). The wild-type mice and hTDP-43 A315T ALS mice injected only with the vehicle (Veh) were included as controls. The median survival time was used to assess the treatment efficacy. The results showed a mild improvement in the median survival for the AAV9-MC5 groups as compared to the vehicle control group (FIG. 7).

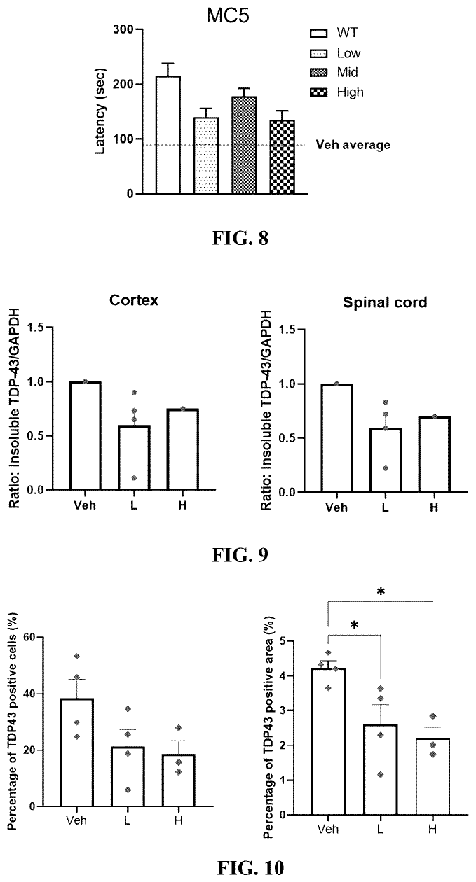

Motor function was evaluated at eight weeks post-injection using the Rotarod test. This test measured the average latency to fall from a rotating rod, enabling a comparison of motor coordination performance between the treated subjects. The results of the Rotarod test are shown as in FIG. 8. The dotted line in FIG. 8 represents the average latency of falling from the rod of the Veh group, which was reduced significantly as compared to the wild-type healthy mice. Compared to the Veh group, the AAV9-MC5 treated mice exhibited a trend of improved motor coordination, as evidenced by the increased latency to fall on the rotarod test (FIG. 8).

At 15 weeks post-AAV administration, the remaining mice in the MC5 treated groups were euthanized, and brain tissue samples were collected from the cortex and spinal cord. Part of the samples was used for the WB analysis, while the remaining samples were fixed in 4% paraformaldehyde (PFA) for the subsequent immunostaining experiments. The WB analysis revealed a modest decrease in the insoluble TDP-43 levels in both the cortex and spinal cord following AAV9-MC5 treatment (FIG. 9). Moreover, the immunostaining showed a significant reduction in the TDP-43 positive area, specifically within the spinal cord after MC5 administration (FIG. 10).

### Example 5. In Vitro Therapeutic Efficacy of the rAAV Candidates Expressing the Optimized GRN and STMN2 in an ALS Cellular Model

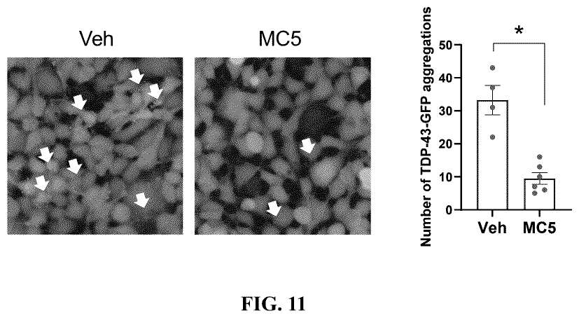

A cell-based system was used to evaluate the effectiveness of the developed rAAV therapies on reducing TDP-43 aggregation. This cellular system employed a cell line engineered with the PiggyBac system to stably express a mutant form of TDP-43 fused to GFP for visualization. A Tet-on promoter controlled the expression of TDP-43, allowing for induction of the TDP-43 aggregates in the cells upon doxycycline treatment. As shown in FIG. 11, treatment with AAV9-MC5 (MOI=1E+6) significantly reduced the levels of the abnormal TDP-43 aggregates.

Sequence information
G1
SEQ ID NO: 1
ATGTGGACCCTCGTTTCTTGGGTCGCCCTGACAGCCGGGCTGGTTGCCGGCACTCGGTGCCCTGACGGACAGTT
TTGCCCCGTGGCTTGTTGTCTTGATCCAGGGGGGGCCAGTTACTCTTGTTGTAGGCCATTGCTTGACAAATGGC
CAACCACTCTTTCCAGGCACCTGGGAGGACCTTGTCAGGTGGACGCTCACTGCAGCGCTGGCCACAGCTGTATT
TTCACAGTGTCTGGGACATCATCCTGTTGTCCTTTCCCCGAAGCGGTGGCTTGTGGCGATGGCCATCATTGCTG
TCCTCGGGGATTTCATTGTAGCGCAGACGGACGCAGCTGCTTTCAACGGTCTGGAAACAATTCCGTCGGGGCCA
TACAATGCCCAGATTCTCAGTTTGAATGCCCCGATTTCTCTACCTGTTGCGTGATGGTGGACGGCTCCTGGGGC
TGCTGTCCTATGCCCCAGGCCTCCTGCTGCGAGGACAGGGTGCATTGTTGTCCTCATGGGGCCTTCTGTGACCT
CGTTCACACAAGGTGTATTACACCAACAGGCACTCACCCTCTGGCCAAGAAACTTCCTGCCCAGAGAACCAATA
GGGCAGTTGCTCTGAGCTCTTCAGTGATGTGCCCTGATGCCCGCTCTCGCTGTCCTGACGGCAGCACATGCTGT
GAACTCCCTTCAGGCAAGTATGGATGTTGTCCTATGCCCAATGCAACTTGCTGTTCCGACCACCTCCATTGCTG
TCCTCAAGATACAGTCTGTGACCTCATACAGTCAAAGTGCCTTTCCAAAGAAAACGCCACAACCGATCTTTTGA
CAAAGCTTCCCGCCCACACAGTTGGCGATGTGAAGTGTGATATGGAGGTGTCCTGCCCTGATGGATATACTTGC
TGCCGCCTGCAAAGTGGAGCATGGGGCTGCTGTCCATTCACCCAAGCCGTGTGCTGCGAGGATCATATCCACTG
TTGCCCTGCTGGGTTTACGTGCGATACTCAAAAAGGCACCTGCGAACAGGGACCACACCAAGTTCCCTGGATGG
AGAAAGCTCCAGCACATCTTTCTTTGCCGGATCCACAGGCCCTCAAGCGGGATGTCCCATGCGATAATGTGTCC
AGCTGCCCGAGTTCTGACACCTGCTGTCAGTTGACTTCTGGAGAGTGGGGATGTTGCCCAATTCCAGAGGCTGT
TTGCTGTTCAGATCATCAACACTGTTGCCCACAGGGTTATACCTGTGTCGCTGAAGGCCAATGTCAACGGGGCT
CTGAGATCGTTGCGGGCCTCGAGAAGATGCCCGCCCGGCGCGCCTCTTTGAGCCACCCAAGAGACATCGGATGC
GACCAGCACACCTCATGTCCTGTCGGACAGACTTGTTGTCCTTCCCTGGGTGGATCTTGGGCGTGTTGTCAACT
CCCCCATGCAGTATGCTGCGAAGATAGGCAACATTGCTGCCCAGCTGGGTATACCTGTAATGTCAAGGCTAGAT
CATGCGAAAAAGAGGTTGTTTCTGCTCAGCCAGCCACATTTCTCGCACGGAGCCCTCATGTGGGGGTTAAAGAC
GTCGAGTGTGGCGAAGGGCATTTCTGTCACGACAATCAAACATGTTGTCGGGATAACCGGCAGGGATGGGCATG
TTGTCCCTATCGGCAAGGCGTGTGCTGTGCCGACAGACGGCATTGCTGTCCAGCTGGGTTCAGATGTGCAGCAA
GAGGGACTAAATGTCTTCGGAGGGAGGCCCCCCGGTGGGATGCACCCCTTCGGGACCCCGCCCTGAGACAGCTG
CTG
G2
SEQ ID NO: 2
ATGTGGACACTCGTTAGTTGGGTTGCCTTGACCGCTGGATTGGTCGCCGGAACACGCTGTCCTGATGGACAGTT
TTGTCCAGTCGCATGCTGTTTGGATCCTGGGGGCGCTAGTTACAGCTGCTGCAGGCCACTGCTCGATAAATGGC
CGACTACATTGTCACGCCATTTGGGAGGGCCTTGCCAAGTCGATGCTCACTGTTCCGCCGGTCATTCTTGCATT
TTCACAGTTTCCGGCACTAGCTCATGCTGCCCGTTTCCGGAAGCCGTTGCCTGTGGCGATGGGCATCATTGCTG
TCCTCGGGGCTTTCACTGCAGCGCCGACGGAAGGTCCTGCTTCCAGAGGTCAGGGAACAATAGCGTTGGAGCGA
TTCAATGCCCTGACTCTCAATTTGAGTGTCCAGATTTTAGCACCTGCTGTGTCATGGTCGATGGATCTTGGGGC
TGTTGCCCCATGCCTCAAGCGTCATGTTGCGAGGATCGCGTCCACTGTTGTCCACACGGAGCCTTTTGTGACCT
GGTACATACTCGGTGCATCACACCAACCGGGACGCACCCTTTGGCCAAAAAGCTTCCCGCTCAGAGAACAAATC
GGGCCGTCGCACTCAGTAGTTCCGTAATGTGTCCCGACGCTCGCTCTCGGTGCCCAGACGGCTCTACATGCTGC
GAGCTGCCGTCAGGGAAGTATGGCTGCTGTCCTATGCCCAACGCTACTTGTTGTTCAGACCACCTCCATTGCTG
TCCTCAGGATACCGTTTGTGATCTCATTCAAAGTAAGTGTCTGTCCAAAGAAAATGCTACTACCGACTTGCTGA
CTAAACTTCCAGCTCATACAGTGGGGGATGTGAAATGCGACATGGAAGTCTCTTGTCCAGACGGGTATACATGC
TGTAGGCTGCAGTCAGGGGCCTGGGGATGCTGTCCTTTTACTCAAGCTGTTTGTTGTGAAGACCATATACACTG
CTGCCCCGCAGGCTTTACCTGTGATACCCAGAAAGGAACATGTGAGCAGGGTCCACATCAAGTGCCTTGGATGG
AGAAAGCCCCCGCTCACCTTTCTCTTCCTGACCCGCAGGCGTTGAAGAGGGACGTGCCTTGTGACAATGTTTCA
TCATGTCCAAGCTCTGACACCTGTTGTCAACTGACATCTGGCGAGTGGGGATGTTGCCCAATCCCAGAAGCCGT
CTGTTGTTCTGATCACCAACACTGTTGCCCACAGGGCTATACCTGCGTGGCCGAAGGTCAATGCCAAAGAGGGA
GCGAGATTGTTGCGGGACTGGAAAAAATGCCCGCTCGGAGGGCCAGCCTGTCCCACCCACGAGACATAGGATGT
GACCAGCATACCTCTTGTCCGGTTGGTCAGACGTGCTGCCCAAGCTTGGGGGGATCTTGGGCATGTTGTCAACT
GCCGCACGCAGTTTGTTGCGAAGACAGACAACACTGCTGTCCCGCGGGCTACACTTGTAACGTTAAGGCAAGAA
GCTGCGAAAAAGAGGTGGTTTCTGCCCAGCCAGCAACCTTCCTCGCTAGATCCCCTCACGTTGGCGTGAAGGAC
GTGGAGTGCGGCGAGGGACATTTCTGTCACGATAACCAAACCTGCTGTCGCGACAACCGCCAGGGTTGGGCCTG
CTGCCCTTATCGCCAGGGAGTGTGCTGTGCCGATCGGCGCCATTGTTGTCCTGCAGGGTTTAGATGCGCTGCTA
GGGGTACTAAATGTCTGAGAAGGGAAGCTCCGAGATGGGATGCCCCTTTGAGAGATCCAGCTCTGAGACAATTG
CTC
G3
SEQ ID NO: 3
ATGTGGACACTTGTTTCATGGGTTGCACTCACCGCTGGACTGGTCGCTGGAACCAGATGTCCAGACGGACAGTT
TTGCCCAGTGGCATGCTGTCTCGATCCTGGCGGAGCTAGCTACTCTTGCTGCAGACCACTGTTGGATAAGTGGC
CAACAACACTGAGCAGACATCTGGGCGGCCCATGCCAAGTGGATGCTCATTGTAGCGCCGGCCACTCCTGCATT
TTCACTGTGTCCGGTACCTCCTCATGCTGCCCATTCCCTGAAGCCGTCGCATGCGGAGATGGGCATCACTGTTG
CCCCAGAGGTTTTCACTGCAGCGCTGATGGGCGGTCTTGCTTCCAAAGGTCCGGAAACAATAGCGTTGGGGCGA
TTCAGTGCCCTGACTCCCAATTTGAATGCCCTGACTTTTCAACCTGTTGTGTAATGGTCGATGGCAGCTGGGGC
TGCTGCCCCATGCCACAGGCTTCTTGTTGCGAAGACCGCGTGCATTGTTGCCCTCATGGAGCGTTTTGTGACCT
CGTGCACACGAGGTGCATTACTCCCACAGGCACTCATCCACTGGCTAAGAAATTGCCTGCCCAGAGAACAAACA
GGGCAGTAGCCTTGAGCTCTTCAGTAATGTGTCCGGACGCCAGATCAAGGTGCCCCGATGGATCTACTTGCTGC
GAGCTCCCCTCCGGCAAGTACGGATGTTGTCCAATGCCAAATGCTACTTGCTGTAGTGACCACCTGCACTGTTG
TCCACAGGATACGGTCTGTGACTTGATACAGTCTAAGTGCCTGTCTAAGGAAAATGCAACAACCGATTTGCTTA
CAAAACTGCCAGCCCATACCGTAGGAGATGTCAAGTGCGACATGGAAGTTAGTTGTCCCGACGGCTATACGTGT
TGTCGACTGCAATCCGGAGCCTGGGGTTGTTGCCCGTTTACTCAGGCTGTATGTTGCGAGGACCACATCCACTG
TTGTCCCGCGGGGTTCACCTGTGACACCCAGAAAGGTACCTGCGAGCAGGGGCCTCATCAGGTACCGTGGATGG
AAAAAGCTCCTGCACACTTGTCACTGCCGGATCCTCAGGCGCTCAAGCGGGATGTCCCTTGCGACAATGTAAGC
TCCTGTCCGTCTAGTGATACGTGTTGCCAACTTACCTCCGGGGAATGGGGCTGTTGCCCTATACCAGAAGCTGT
CTGTTGCTCTGATCATCAACATTGCTGCCCCCAGGGTTACACATGTGTAGCAGAGGGTCAGTGTCAAAGAGGGT
CAGAAATTGTAGCTGGCTTGGAAAAAATGCCCGCAAGGCGCGCATCCCTGTCTCACCCTCGGGATATAGGATGC
GATCAGCACACCTCTTGTCCAGTGGGTCAGACTTGTTGTCCCAGCTTGGGGGGCAGCTGGGCTTGTTGTCAATT
GCCTCACGCAGTTTGCTGTGAAGACCGCCAACATTGCTGTCCTGCTGGATACACATGCAATGTCAAGGCTAGGA
GCTGTGAAAAAGAGGTGGTGTCTGCACAGCCCGCAACCTTTCTTGCACGCTCTCCTCATGTCGGAGTGAAAGAT
GTTGAATGCGGAGAAGGACACTTTTGCCATGACAATCAGACTTGTTGCAGGGACAACAGACAAGGATGGGCTTG
TTGCCCTTATCGCCAAGGAGTATGCTGTGCGGATAGAAGACATTGTTGCCCGGCCGGGTTTAGGTGTGCTGCTA
GAGGCACCAAATGTCTGAGGCGAGAAGCACCTAGATGGGACGCTCCACTGCGGGACCCTGCCTTGAGACAACTC
CTG
G4
SEQ ID NO: 4
ATGTGGACCCTCGTGTCCTGGGTGGCACTGACAGCCGGGTTGGTGGCTGGAACTAGATGCCCAGACGGACAATT
CTGTCCCGTGGCTTGTTGCTTGGACCCTGGAGGCGCATCCTATAGCTGTTGCAGGCCCTTGCTGGACAAATGGC
CAACCACACTGTCTCGGCATTTGGGCGGCCCATGCCAGGTGGATGCTCATTGCAGCGCCGGCCACAGCTGCATC
TTCACCGTCTCCGGAACAAGCAGCTGTTGTCCATTTCCAGAAGCGGTCGCTTGTGGCGATGGACATCACTGTTG
TCCTCGCGGATTTCACTGCTCTGCCGATGGAAGGTCCTGCTTTCAACGGAGCGGAAATAATTCTGTCGGGGCCA
TTCAATGTCCCGACTCCCAGTTCGAGTGCCCTGATTTTAGCACATGTTGTGTCATGGTCGATGGCAGCTGGGGA
TGCTGCCCAATGCCACAGGCATCCTGCTGCGAAGATAGGGTACATTGTTGCCCTCATGGAGCCTTTTGTGATCT
CGTTCATACAAGGTGTATTACTCCAACTGGCACACATCCCCTCGCCAAGAAATTGCCTGCACAGAGAACCAACC
GGGCTGTGGCCCTCTCTTCCTCTGTTATGTGTCCAGACGCACGGTCTAGATGTCCCGATGGATCTACATGTTGC
GAACTGCCTAGCGGAAAGTATGGGTGCTGTCCCATGCCTAACGCTACGTGCTGCTCTGATCATTTGCACTGCTG
TCCACAGGATACAGTGTGTGATCTGATTCAAAGCAAATGCCTGTCCAAAGAAAATGCAACAACCGATCTCTTGA
CTAAACTCCCTGCCCACACCGTGGGCGACGTAAAGTGTGACATGGAGGTCTCTTGCCCAGACGGCTATACATGC
TGTCGGCTGCAGTCTGGAGCCTGGGGCTGTTGCCCCTTCACTCAGGCCGTCTGTTGCGAGGATCATATCCATTG
TTGTCCAGCAGGATTTACTTGCGACACCCAGAAAGGCACCTGCGAGCAAGGCCCACACCAAGTGCCTTGGATGG
AGAAGGCCCCAGCTCACCTCAGCCTGCCTGACCCTCAGGCACTCAAGAGGGATGTGCCATGTGACAATGTTTCT
TCCTGCCCTTCCAGCGATACATGTTGTCAATTGACATCTGGCGAATGGGGATGTTGTCCCATCCCAGAGGCTGT
GTGCTGCTCCGATCACCAGCACTGTTGTCCACAGGGGTATACATGTGTTGCTGAAGGCCAATGTCAACGGGGCT
CCGAAATCGTGGCAGGACTCGAAAAAATGCCTGCAAGGAGAGCTAGCCTCTCTCATCCACGCGATATCGGATGC
GACCAACATACATCCTGCCCAGTCGGCCAAACTTGCTGTCCCTCACTTGGAGGTTCCTGGGCTTGCTGCCAGCT
GCCCCATGCCGTGTGCTGCGAAGATAGACAGCACTGTTGCCCTGCCGGGTACACCTGCAATGTCAAGGCCAGAT
CATGCGAAAAAGAGGTGGTCAGCGCACAGCCAGCTACATTCTTGGCCAGATCCCCACATGTGGGCGTGAAAGAC
GTCGAATGCGGCGAAGGCCATTTCTGCCACGACAACCAAACCTGCTGCAGGGACAACAGGCAGGGATGGGCTTG
CTGTCCATATAGACAAGGGGTGTGCTGCGCAGACAGGAGACATTGCTGCCCTGCTGGGTTCCGGTGCGCTGCAA
GGGGAACTAAGTGCCTGAGACGGGAGGCACCTCGGTGGGATGCCCCACTGAGGGACCCCGCTTTGAGACAGCTC
CTG
G5
SEQ ID NO: 5
ATGTGGACCCTGGTGTCTTGGGTGGCCCTGACCGCCGGTCTGGTGGCCGGAACCAGATGCCCCGATGGCCAGTT
CTGCCCCGTGGCTTGCTGTCTGGACCCCGGCGGAGCATCTTATAGCTGCTGCAGACCTCTGCTGGATAAGTGGC
CTACAACTCTGTCCCGGCACCTCGGCGGCCCTTGCCAAGTGGACGCCCACTGTAGTGCCGGACACTCTTGTATC
TTCACCGTATCTGGAACCAGCAGCTGTTGCCCTTTCCCCGAGGCCGTGGCCTGTGGCGACGGTCATCACTGCTG
CCCTCGGGGCTTCCACTGTTCTGCTGATGGCAGAAGCTGCTTCCAGAGAAGCGGCAACAACAGCGTCGGAGCCA
TTCAGTGTCCGGACAGCCAGTTTGAGTGCCCTGACTTCAGCACATGCTGCGTGATGGTCGACGGCTCCTGGGGC
TGCTGCCCTATGCCCCAGGCCAGCTGCTGCGAGGACAGAGTGCACTGCTGCCCTCACGGCGCTTTTTGTGATCT
GGTCCACACAAGATGCATCACACCAACCGGCACACACCCTCTGGCCAAGAAACTGCCTGCCCAGCGGACCAACC
GGGCCGTTGCCCTGAGCAGCAGCGTGATGTGCCCAGATGCCAGATCCCGGTGCCCTGACGGCAGCACCTGTTGT
GAACTGCCTTCTGGCAAATACGGCTGCTGTCCCATGCCCAATGCTACGTGCTGCTCCGACCACCTGCACTGCTG
CCCTCAGGACACAGTGTGCGACCTGATCCAAAGCAAGTGCCTGAGCAAAGAGAACGCCACAACAGACCTGCTGA
CCAAGCTGCCTGCTCATACCGTGGGCGATGTGAAGTGCGATATGGAAGTGTCCTGCCCCGACGGCTACACCTGT
TGTAGACTGCAGAGCGGCGCCTGGGGCTGCTGTCCTTTCACCCAGGCCGTCTGCTGTGAGGATCACATCCACTG
TTGCCCTGCCGGCTTCACCTGCGACACCCAGAAGGGCACCTGCGAGCAGGGCCCCCACCAGGTGCCCTGGATGG
AAAAGGCCCCAGCTCATCTGAGCCTGCCTGACCCTCAAGCTCTGAAGCGGGACGTGCCATGTGATAACGTGTCC
AGCTGTCCTTCTAGCGATACCTGTTGCCAACTGACCAGCGGCGAGTGGGGATGCTGTCCTATCCCCGAGGCCGT
GTGCTGCAGCGACCACCAGCACTGCTGTCCCCAGGGCTACACCTGTGTGGCCGAGGGCCAGTGCCAGAGAGGAA
GCGAGATCGTGGCTGGCCTGGAAAAGATGCCTGCCCGACGGGCCAGCCTGAGCCACCCCAGAGACATCGGCTGT
GACCAGCACACCAGCTGCCCAGTGGGACAGACCTGCTGCCCTAGCCTGGGCGGCTCTTGGGCCTGCTGCCAGCT
GCCCCACGCCGTGTGCTGCGAGGACAGACAGCACTGCTGCCCTGCCGGATACACCTGCAACGTGAAGGCCCGGA
GCTGCGAGAAGGAAGTGGTGAGCGCTCAACCTGCCACCTTCCTGGCCAGATCCCCTCATGTGGGCGTTAAGGAC
GTGGAATGCGGGGAAGGCCACTTCTGCCACGACAACCAGACATGCTGCAGAGATAATAGACAGGGATGGGCCTG
CTGTCCCTACAGACAGGGCGTGTGTTGCGCCGACCGGAGACACTGTTGCCCAGCCGGCTTTAGATGCGCCGCAC
GGGGCACAAAATGCCTGAGAAGGGAAGCCCCTAGATGGGACGCCCCTTTGAGAGATCCTGCCCTGCGCCAGCTG
CTCTGA
G6
SEQ ID NO: 6
ATGTGGACACTGGTGAGCTGGGTGGCCCTTACAGCCGGCCTGGTGGCCGGCACAAGATGCCCTGATGGACAATT
TTGCCCAGTGGCCTGCTGCCTGGACCCTGGCGGAGCCAGCTACAGCTGCTGCAGACCCCTCCTGGATAAGTGGC
CCACAACCCTGTCTAGACACCTGGGCGGACCTTGCCAGGTGGACGCCCACTGCTCTGCCGGCCACAGCTGCATC
TTCACCGTGTCTGGAACATCTAGCTGTTGTCCCTTCCCTGAGGCTGTTGCCTGTGGCGATGGCCACCACTGCTG
TCCTAGAGGCTTCCATTGCTCTGCCGATGGTAGATCCTGCTTCCAGAGATCAGGCAACAACAGCGTGGGAGCCA
TCCAGTGCCCTGATAGCCAGTTCGAGTGCCCAGACTTCAGCACTTGCTGTGTGATGGTCGATGGCTCTTGGGGC
TGTTGTCCCATGCCCCAGGCTTCTTGCTGCGAAGATAGAGTGCACTGCTGCCCCCACGGCGCCTTCTGTGACCT
GGTCCACACCAGATGCATCACCCCCACCGGCACACACCCCCTGGCAAAGAAGCTGCCTGCTCAGAGAACCAATA
GAGCTGTGGCCCTGAGCTCTAGCGTGATGTGCCCTGATGCCAGATCCAGATGTCCTGATGGCAGCACCTGCTGC
GAGCTGCCATCTGGCAAATATGGCTGTTGTCCAATGCCTAACGCCACCTGCTGTTCTGACCACCTGCACTGCTG
CCCTCAAGACACCGTGTGCGACCTGATCCAGAGCAAATGCCTCTCCAAGGAAAACGCCACAACCGACCTGCTGA
CCAAGCTGCCTGCCCACACAGTGGGCGATGTGAAGTGTGATATGGAAGTGAGCTGCCCTGATGGCTACACCTGT
TGCAGACTGCAGTCTGGCGCCTGGGGCTGCTGTCCTTTCACCCAGGCCGTGTGTTGCGAGGACCACATCCACTG
CTGCCCTGCTGGATTTACCTGTGACACACAGAAGGGCACCTGTGAACAGGGCCCTCACCAGGTGCCATGGATGG
AAAAGGCCCCTGCTCACCTGAGCCTGCCAGACCCTCAGGCCCTGAAGAGGGATGTCCCCTGCGACAATGTGAGC
TCCTGCCCTAGCTCTGATACATGCTGCCAGCTGACCAGCGGAGAGTGGGGATGCTGCCCCATCCCTGAGGCCGT
GTGCTGCTCCGACCACCAACATTGTTGCCCCCAGGGCTATACCTGTGTGGCCGAGGGCCAATGTCAAAGAGGTA
GCGAAATTGTGGCCGGCCTGGAGAAGATGCCTGCCAGAAGAGCCAGCCTGAGCCACCCTAGAGACATCGGCTGT
GACCAGCACACCAGCTGTCCTGTGGGCCAGACATGCTGCCCTAGCCTGGGCGGCAGCTGGGCCTGCTGCCAGCT
GCCTCACGCCGTCTGTTGTGAGGATAGACAGCACTGCTGTCCTGCTGGCTACACATGCAATGTGAAAGCCAGGT
CCTGTGAGAAAGAGGTGGTGAGCGCCCAGCCTGCTACCTTTCTGGCCAGAAGCCCTCATGTGGGCGTGAAGGAT
GTGGAATGTGGCGAGGGCCATTTCTGTCACGACAACCAGACCTGTTGTAGAGACAACAGACAGGGATGGGCTTG
CTGCCCATACAGACAGGGCGTTTGCTGTGCCGACAGAAGACACTGCTGCCCTGCCGGATTCAGATGCGCTGCCA
GAGGGACCAAGTGTCTGAGAAGAGAAGCCCCTAGATGGGACGCCCCTCTGAGGGACCCTGCTCTCAGACAGCTG
CTGTGA
G7
SEQ ID NO: 7
ATGTGGACCCTGGTGTCCTGGGTCGCCCTGACCGCTGGCCTCGTGGCCGGCACAAGATGCCCCGACGGGCAGTT
CTGTCCTGTGGCTTGCTGCCTGGACCCCGGCGGCGCTAGCTACAGCTGCTGCAGACCCCTGCTGGACAAATGGC
CTACCACACTGTCCCGGCATCTCGGCGGCCCCTGTCAAGTCGACGCCCACTGCAGCGCCGGCCACAGCTGCATC
TTCACCGTCTCCGGCACAAGCAGCTGCTGCCCCTTCCCTGAGGCCGTGGCCTGTGGCGATGGGCACCACTGCTG
CCCTAGAGGCTTCCACTGTAGCGCCGATGGCAGAAGCTGCTTTCAGAGAAGCGGCAACAACAGCGTGGGCGCCA
TTCAGTGCCCCGACAGCCAATTCGAGTGCCCCGACTTCAGCACCTGCTGCGTGATGGTCGATGGCAGCTGGGGG
TGCTGCCCCATGCCCCAAGCTAGCTGCTGTGAAGACAGAGTGCATTGCTGCCCCCACGGCGCCTTCTGCGACCT
GGTGCACACAAGATGCATCACCCCCACCGGCACCCACCCCCTGGCCAAGAAGCTGCCCGCCCAAAGAACCAACA
GAGCCGTGGCCCTGAGCAGCAGCGTGATGTGCCCCGACGCTAGAAGCAGATGTCCCGACGGCAGCACATGCTGC
GAGCTGCCTAGCGGCAAGTACGGCTGCTGCCCCATGCCCAATGCTACCTGCTGCAGCGATCATCTGCACTGTTG
CCCCCAAGACACCGTGTGCGACCTGATTCAGAGCAAATGCCTGTCCAAGGAGAACGCCACCACCGACCTGCTGA
CCAAGCTGCCTGCCCATACCGTGGGCGACGTGAAGTGCGACATGGAGGTGAGCTGCCCCGACGGCTACACATGC
TGCAGACTGCAGAGCGGCGCCTGGGGGTGCTGCCCTTTCACCCAAGCCGTGTGCTGCGAGGACCACATCCACTG
TTGCCCTGCTGGCTTCACCTGTGACACACAGAAGGGCACCTGCGAGCAAGGCCCTCACCAAGTGCCTTGGATGG
AGAAGGCCCCCGCCCACCTCAGCCTGCCCGACCCCCAAGCCCTGAAGAGAGACGTGCCCTGCGACAACGTGAGC
AGCTGCCCTAGCTCCGACACCTGCTGTCAGCTCACAAGCGGCGAGTGGGGCTGCTGTCCCATCCCCGAGGCTGT
GTGCTGCAGCGACCACCAACACTGCTGCCCCCAAGGCTACACATGTGTCGCCGAGGGGCAGTGTCAGAGAGGCA
GCGAGATCGTGGCCGGCCTGGAGAAGATGCCCGCTAGAAGAGCTAGCCTGAGCCACCCTAGAGACATCGGCTGC
GATCAGCATACAAGCTGCCCCGTGGGGCAGACCTGCTGCCCTTCCCTGGGCGGCAGCTGGGCTTGTTGCCAACT
GCCTCACGCCGTGTGTTGCGAAGACAGACAGCACTGCTGCCCCGCCGGCTACACATGTAATGTGAAGGCTAGAA
GCTGCGAGAAAGAGGTGGTGAGCGCTCAGCCCGCCACCTTCCTGGCTAGAAGCCCCCACGTGGGCGTGAAGGAC
GTGGAGTGCGGCGAGGGCCACTTCTGCCACGACAATCAGACCTGTTGTCGGGACAACAGACAAGGCTGGGCTTG
CTGCCCCTACCGGCAAGGCGTGTGCTGCGCCGACAGAAGACACTGCTGTCCTGCCGGCTTCAGATGCGCCGCTA
GAGGCACCAAGTGCCTGCGGAGAGAGGCCCCTAGATGGGACGCCCCCCTGAGAGACCCCGCCCTGAGACAGCTG
CTGTGA
G8
SEQ ID NO: 8
ATGTGGACCCTGGTGAGCTGGGTGGCTCTGACAGCTGGCCTTGTGGCTGGAACTAGATGTCCCGATGGACAATT
CTGCCCCGTGGCCTGCTGTCTGGATCCCGGAGGCGCTAGCTATAGCTGTTGTAGACCCCTGCTGGATAAGTGGC
CCACCACCCTGAGCAGACACCTGGGCGGACCTTGTCAAGTGGACGCTCATTGCAGCGCTGGACACAGCTGCATT
TTCACCGTGAGCGGCACCAGCAGCTGCTGCCCTTTTCCTGAAGCTGTGGCCTGCGGGGACGGCCACCATTGTTG
TCCTAGAGGATTTCACTGCAGCGCCGACGGCAGAAGCTGCTTCCAAAGAAGCGGCAACAACAGCGTGGGCGCCA
TCCAGTGTCCCGACAGCCAATTTGAGTGCCCCGACTTCAGCACCTGCTGCGTGATGGTGGACGGCAGCTGGGGA
TGTTGTCCTATGCCTCAAGCCAGCTGCTGTGAGGACAGAGTGCACTGCTGCCCCCACGGCGCTTTTTGCGATCT
GGTGCACACCAGATGCATCACCCCCACCGGCACACATCCCCTGGCTAAAAAGCTGCCTGCCCAAAGAACAAACA
GAGCCGTGGCCCTGAGCAGCAGCGTGATGTGTCCTGATGCCAGAAGCAGATGCCCCGACGGCAGCACATGTTGT
GAGCTGCCCAGCGGCAAATACGGCTGCTGCCCTATGCCCAACGCCACCTGTTGTAGCGACCATCTGCACTGCTG
TCCCCAGGACACAGTGTGCGACCTGATCCAGAGCAAGTGCCTGAGCAAGGAGAACGCCACCACCGACCTGCTGA
CCAAGCTGCCCGCTCATACCGTGGGCGATGTGAAGTGCGATATGGAGGTGAGCTGCCCCGACGGCTATACCTGT
TGCAGACTGCAAAGCGGCGCCTGGGGCTGTTGTCCCTTTACCCAAGCTGTGTGTTGCGAGGACCACATCCACTG
CTGCCCCGCTGGATTTACCTGCGACACACAGAAAGGCACCTGCGAGCAGGGACCCCATCAGGTGCCTTGGATGG
AAAAAGCCCCCGCTCACCTGAGCCTGCCCGATCCTCAAGCTCTTAAAAGAGATGTGCCCTGCGATAACGTGAGC
AGCTGCCCCAGCAGCGACACCTGTTGTCAACTGACCAGCGGCGAATGGGGCTGCTGCCCTATTCCTGAGGCCGT
GTGCTGTAGCGACCACCAGCATTGTTGCCCCCAGGGCTATACATGCGTGGCCGAGGGACAATGCCAGAGAGGAA
GCGAAATTGTGGCTGGCCTGGAAAAGATGCCCGCCAGAAGAGCCAGCCTGAGCCACCCTAGAGATATCGGCTGT
GACCAGCACACCAGCTGCCCCGTTGGACAAACATGTTGTCCTAGCCTGGGCGGAAGCTGGGCCTGTTGTCAACT
GCCTCACGCCGTGTGTTGCGAAGATAGACAGCACTGCTGCCCCGCCGGATACACCTGCAATGTGAAGGCCAGAA
GCTGCGAGAAGGAGGTGGTGAGCGCCCAGCCTGCTACATTTCTGGCCAGAAGCCCCCATGTGGGCGTGAAGGAT
GTGGAGTGTGGCGAGGGACATTTCTGCCATGATAACCAGACCTGCTGCAGAGACAACAGACAGGGCTGGGCCTG
CTGCCCCTATAGACAAGGAGTGTGTTGCGCTGACAGAAGACACTGCTGTCCCGCCGGCTTTAGATGCGCCGCTA
GAGGCACAAAGTGCCTGAGAAGAGAGGCCCCCAGATGGGACGCCCCTCTTAGAGATCCTGCTCTGAGACAACTG
CTGTGA
G0
SEQ ID NO: 9
ATGTGGACCCTGGTGAGCTGGGTGGCCTTAACAGCAGGGCTGGTGGCTGGAACGCGGTGCCCAGATGGTCAGTT
CTGCCCTGTGGCCTGCTGCCTGGACCCCGGAGGAGCCAGCTACAGCTGCTGCCGTCCCCTTCTGGACAAATGGC
CCACAACACTGAGCAGGCATCTGGGTGGCCCCTGCCAGGTTGATGCCCACTGCTCTGCCGGCCACTCCTGCATC
TTTACCGTCTCAGGGACTTCCAGTTGCTGCCCCTTCCCAGAGGCCGTGGCATGCGGGGATGGCCATCACTGCTG
CCCACGGGGCTTCCACTGCAGTGCAGACGGGCGATCCTGCTTCCAAAGATCAGGTAACAACTCCGTGGGTGCCA
TCCAGTGCCCTGATAGTCAGTTCGAATGCCCGGACTTCTCCACGTGCTGTGTTATGGTCGATGGCTCCTGGGGG
TGCTGCCCCATGCCCCAGGCTTCCTGCTGTGAAGACAGGGTGCACTGCTGTCCGCACGGTGCCTTCTGCGACCT
GGTTCACACCCGCTGCATCACACCCACGGGCACCCACCCCCTGGCAAAGAAGCTCCCTGCCCAGAGGACTAACA
GGGCAGTGGCCTTGTCCAGCTCGGTCATGTGTCCGGACGCACGGTCCCGGTGCCCTGATGGTTCTACCTGCTGT
GAGCTGCCCAGTGGGAAGTATGGCTGCTGCCCAATGCCCAACGCCACCTGCTGCTCCGATCACCTGCACTGCTG
CCCCCAAGACACTGTGTGTGACCTGATCCAGAGTAAGTGCCTCTCCAAGGAGAACGCTACCACGGACCTCCTCA
CTAAGCTGCCTGCGCACACAGTGGGGGATGTGAAATGTGACATGGAGGTGAGCTGCCCAGATGGCTATACCTGC
TGCCGTCTACAGTCGGGGGCCTGGGGCTGCTGCCCTTTTACCCAGGCTGTGTGCTGTGAGGACCACATACACTG
CTGTCCCGCGGGGTTTACGTGTGACACGCAGAAGGGTACCTGTGAACAGGGGCCCCACCAGGTGCCCTGGATGG
AGAAGGCCCCAGCTCACCTCAGCCTGCCAGACCCACAAGCCTTGAAGAGAGATGTCCCCTGTGATAATGTCAGC
AGCTGTCCCTCCTCCGATACCTGCTGCCAACTCACGTCTGGGGAGTGGGGCTGCTGTCCAATCCCAGAGGCTGT
CTGCTGCTCGGACCACCAGCACTGCTGCCCCCAGGGCTACACGTGTGTAGCTGAGGGGCAGTGTCAGCGAGGAA
GCGAGATCGTGGCTGGACTGGAGAAGATGCCTGCCCGCCGGGCTTCCTTATCCCACCCCAGAGACATCGGCTGT
GACCAGCACACCAGCTGCCCGGTGGGGCAGACCTGCTGCCCGAGCCTGGGTGGGAGCTGGGCCTGCTGCCAGTT
GCCCCATGCTGTGTGCTGCGAGGATCGCCAGCACTGCTGCCCGGCTGGCTACACCTGCAACGTGAAGGCTCGAT
CCTGCGAGAAGGAAGTGGTCTCTGCCCAGCCTGCCACCTTCCTGGCCCGTAGCCCTCACGTGGGTGTGAAGGAC
GTGGAGTGTGGGGAAGGACACTTCTGCCATGATAACCAGACCTGCTGCCGAGACAACCGACAGGGCTGGGCCTG
CTGTCCCTACCGCCAGGGCGTCTGTTGTGCTGATCGGCGCCACTGCTGTCCTGCTGGCTTCCGCTGCGCAGCCA
GGGGTACCAAGTGTTTGCGCAGGGAGGCCCCGCGCTGGGACGCCCCTTTGAGGGACCCAGCCTTGAGACAGCTG
CTGTGA
WT protein sequence of progranulin (PGRN)
SEQ ID NO: 10
MWTLVSWVALTAGLVAGTRCPDGQFCPVACCLDPGGASYSCCRPLLDKWPTTLSRHLGGPCQVDAHCSAGHSCI
FTVSGTSSCCPFPEAVACGDGHHCCPRGFHCSADGRSCFQRSGNNSVGAIQCPDSQFECPDFSTCCVMVDGSWG
CCPMPQASCCEDRVHCCPHGAFCDLVHTRCITPTGTHPLAKKLPAQRTNRAVALSSSVMCPDARSRCPDGSTCC
ELPSGKYGCCPMPNATCCSDHLHCCPQDTVCDLIQSKCLSKENATTDLLTKLPAHTVGDVKCDMEVSCPDGYTC
CRLQSGAWGCCPFTQAVCCEDHIHCCPAGFTCDTQKGTCEQGPHQVPWMEKAPAHLSLPDPQALKRDVPCDNVS
SCPSSDTCCQLTSGEWGCCPIPEAVCCSDHQHCCPQGYTCVAEGQCQRGSEIVAGLEKMPARRASLSHPRDIGC
DQHTSCPVGQTCCPSLGGSWACCQLPHAVCCEDRQHCCPAGYTCNVKARSCEKEVVSAQPATFLARSPHVGVKD
VECGEGHFCHDNQTCCRDNRQGWACCPYRQGVCCADRRHCCPAGFRCAARGTKCLRREAPRWDAPLRDPALRQL
L
S1
SEQ ID NO: 11
ATGGCAAAGACTGCTATGGCTTATAAGGAGAAAATGAAGGAACTCTCTATGCTCTCTCTCATTTGTTCCTGCTT
CTATCCTGAGCCTCGGAACATTAATATCTACACTTACGATGATATGGAGGTAAAGCAAATTAACAAGCGAGCGA
GTGGGCAGGCCTTCGAACTTATATTGAAGCCACCCTCCCCTATATCCGAAGCGCCGAGGACATTGGCCAGCCCA
AAAAAGAAAGACCTTTCCTTGGAGGAGATTCAAAAGAAGTTGGAGGCTGCGGAGGAAAGGAGAAAGTCTCAAGA
GGCCCAAGTCTTGAAGCAGCTCGCCGAAAAGAGGGAGCACGAAAGGGAGGTTCTCCAGAAAGCTCTCGAGGAAA
ACAATAACTTTTCAAAAATGGCGGAGGAAAAACTCATCCTTAAGATGGAGCAAATTAAAGAAAATCGGGAGGCT
AACCTCGCCGCAATAATCGAAAGGTTGCAAGAGAAGCTCGTCAAGTTTATTTCCTCAGAGCTTAAAGAATCAAT
AGAGTCCCAGTTTTTGGAGCTCCAAAGAGAAGGCGAGAAGCAG
S2
SEQ ID NO: 12
ATGGCAAAGACAGCTATGGCATATAAGGAGAAAATGAAAGAACTGAGCATGCTGTCTCTGATATGTTCATGTTT
TTATCCTGAGCCACGCAATATTAACATTTATACATACGACGACATGGAGGTCAAACAAATAAATAAAAGAGCTT
CTGGACAAGCATTCGAACTCATACTCAAGCCACCCTCACCCATTTCTGAAGCCCCTAGGACTTTGGCATCACCA
AAAAAGAAAGACCTCAGCCTTGAAGAGATACAGAAGAAACTGGAAGCTGCAGAAGAGAGGCGCAAATCCCAGGA
GGCCCAAGTTCTGAAACAGCTCGCCGAGAAACGGGAACATGAAAGGGAAGTTCTCCAGAAGGCTCTGGAGGAGA
ACAACAATTTCTCTAAGATGGCTGAGGAGAAGTTGATCTTGAAGATGGAACAGATAAAGGAGAATAGAGAGGCG
AACCTGGCCGCCATCATAGAACGGCTGCAGGAAAAGCTGGTGAAGTTTATTAGCTCCGAACTCAAAGAAAGCAT
AGAATCCCAGTTTCTGGAACTGCAGCGGGAAGGTGAAAAACAG
S3
SEQ ID NO: 13
ATGGCTAAGACAGCAATGGCTTATAAAGAGAAGATGAAAGAGCTCAGCATGTTGTCCTTGATTTGCTCTTGTTT
CTACCCAGAACCACGGAACATAAACATATATACATATGATGATATGGAAGTGAAGCAAATCAATAAAAGGGCCT
CTGGTCAAGCGTTTGAACTGATCTTGAAGCCTCCCTCCCCAATTAGCGAGGCCCCAAGGACCCTCGCTTCTCCC
AAAAAAAAGGACCTCTCTCTGGAGGAGATCCAAAAGAAGTTGGAGGCAGCCGAGGAAAGGCGCAAATCCCAGGA
AGCGCAAGTCCTGAAGCAGCTCGCCGAAAAGAGGGAGCACGAGCGGGAGGTCCTCCAAAAAGCACTCGAAGAAA
ATAATAACTTTTCTAAGATGGCTGAGGAAAAATTGATACTCAAGATGGAGCAGATTAAAGAAAATAGAGAAGCT
AACCTGGCAGCCATTATAGAGAGGCTCCAGGAGAAGTTGGTGAAGTTTATCAGCTCTGAACTGAAAGAGTCAAT
CGAATCTCAATTTCTTGAGCTCCAGAGGGAAGGAGAAAAGCAA
S4
SEQ ID NO: 14
ATGGCCAAGACCGCTATGGCCTATAAGGAAAAGATGAAGGAGCTCTCCATGCTGAGCCTCATTTGTAGCTGCTT
CTACCCTGAGCCCCGGAACATTAACATCTACACCTATGACGACATGGAAGTCAAACAGATAAATAAGCGGGCAT
CCGGCCAGGCCTTCGAACTGATCCTCAAGCCACCTTCTCCTATTTCCGAAGCTCCCAGGACCTTGGCCTCCCCC
AAGAAAAAGGATCTGAGCCTCGAAGAGATTCAGAAGAAGCTGGAAGCTGCTGAGGAGCGGAGGAAGAGCCAGGA
GGCGCAGGTGCTGAAGCAGCTCGCCGAGAAGAGGGAGCATGAACGGGAGGTCTTGCAGAAGGCCCTCGAAGAGA
ACAATAACTTCTCCAAGATGGCTGAGGAAAAGCTCATCCTCAAGATGGAGCAAATCAAAGAGAACAGAGAGGCC
AACCTGGCTGCAATCATCGAGAGGCTCCAGGAAAAGCTCGTGAAATTCATTTCTAGCGAGCTCAAAGAATCTAT
CGAGTCCCAGTTTTTGGAACTCCAGAGGGAAGGCGAAAAACAA
S5
SEQ ID NO: 15
ATGGCCAAGACCGCCATGGCCTACAAGGAGAAGATGAAAGAGCTGAGCATGCTGTCTCTGATCTGCAGCTGTTT
CTACCCCGAGCCTCGGAACATCAACATCTATACATACGACGATATGGAAGTGAAACAGATCAACAAGCGGGCCA
GCGGCCAGGCCTTCGAGCTCATCCTGAAACCTCCTAGCCCCATCTCTGAGGCCCCTAGAACCCTGGCCTCTCCA
AAGAAGAAGGACCTGTCCCTGGAAGAGATCCAGAAGAAGCTGGAGGCTGCTGAGGAACGCAGAAAGTCCCAGGA
GGCCCAGGTCCTGAAGCAGCTGGCAGAAAAAAGAGAGCACGAGAGAGAGGTGCTGCAGAAAGCCCTGGAAGAGA
ACAACAATTTCAGCAAGATGGCCGAGGAAAAGCTGATTCTGAAGATGGAACAGATCAAGGAAAATAGAGAAGCC
AACCTGGCCGCTATCATCGAGCGGCTGCAGGAGAAGCTGGTGAAGTTCATCAGCAGCGAGCTGAAGGAAAGCAT
CGAGAGCCAATTTCTGGAACTGCAAAGAGAAGGCGAGAAGCAGTGA
S6
SEQ ID NO: 16
ATGGCCAAGACCGCCATGGCCTATAAGGAAAAGATGAAGGAACTGAGCATGCTGAGCCTGATCTGTAGCTGCTT
CTACCCTGAGCCCAGAAACATCAACATCTACACCTACGATGACATGGAAGTGAAACAGATCAACAAGAGAGCCA
GCGGCCAGGCCTTCGAGCTGATCCTGAAACCTCCATCTCCTATCAGCGAGGCCCCTAGAACACTGGCTAGCCCT
AAGAAAAAGGATCTGTCTCTGGAAGAAATCCAGAAAAAGCTGGAAGCAGCTGAGGAAAGAAGAAAGTCCCAGGA
GGCCCAGGTCCTGAAGCAGCTGGCCGAGAAGAGAGAGCACGAGAGAGAGGTGCTGCAGAAGGCCCTGGAAGAGA
ACAACAATTTCAGCAAGATGGCTGAAGAGAAACTGATTCTGAAGATGGAACAGATCAAGGAAAATAGAGAGGCC
AACCTGGCCGCCATCATCGAGAGGCTGCAGGAGAAGCTCGTGAAGTTCATCTCCAGCGAGCTGAAGGAGAGCAT
CGAGTCTCAATTTCTGGAACTTCAAAGAGAGGGCGAGAAGCAGTGA
S7
SEQ ID NO: 17
ATGGCCAAGACCGCCATGGCCTACAAGGAGAAGATGAAGGAGCTGAGCATGCTGAGCCTGATCTGCAGCTGCTT
CTACCCTGAGCCTAGAAACATCAACATCTACACCTACGATGACATGGAGGTGAAGCAGATCAACAAGAGAGCTA
GCGGCCAAGCCTTCGAGCTGATCCTGAAGCCCCCTAGCCCCATCAGCGAGGCCCCTAGAACCCTGGCTAGCCCC
AAGAAAAAGGACCTGAGCCTGGAGGAGATTCAGAAGAAGCTGGAGGCCGCCGAGGAGAGAAGAAAGAGCCAAGA
GGCCCAAGTGCTGAAGCAGCTGGCCGAGAAGAGAGAGCACGAGAGAGAGGTGCTGCAGAAGGCCCTGGAGGAGA
ACAATAACTTCAGCAAGATGGCCGAGGAGAAGCTCATCCTGAAGATGGAGCAGATCAAGGAGAACAGAGAGGCC
AACCTGGCCGCCATCATCGAGAGACTGCAAGAGAAGCTGGTGAAGTTCATCAGCAGCGAGCTGAAGGAGAGCAT
CGAGAGCCAATTCCTGGAGCTGCAGAGAGAGGGCGAGAAGCAGTGA
S8
SEQ ID NO: 18
ATGGCCAAGACCGCCATGGCCTACAAGGAGAAGATGAAGGAGCTGAGCATGCTGAGCCTGATCTGCAGCTGCTT
CTACCCCGAGCCCAGAAACATCAACATCTACACCTACGATGACATGGAGGTGAAGCAGATCAACAAGAGAGCCA
GCGGCCAGGCCTTCGAGCTGATTCTGAAACCTCCCAGCCCCATCAGCGAGGCCCCTAGAACACTGGCTAGCCCT
AAGAAAAAGGACCTGAGCCTGGAGGAGATCCAGAAGAAGCTGGAGGCCGCCGAGGAGAGAAGAAAGAGCCAGGA
GGCCCAGGTGCTGAAGCAGCTGGCTGAGAAAAGAGAGCACGAGAGAGAGGTGCTGCAGAAGGCCCTGGAGGAGA
ACAACAACTTCAGCAAGATGGCCGAGGAGAAGCTGATCCTGAAGATGGAGCAGATCAAGGAGAACAGAGAGGCC
AACCTGGCCGCCATCATCGAGAGACTGCAGGAGAAGCTGGTGAAGTTCATCAGCAGCGAGCTGAAGGAGAGCAT
CGAGAGCCAGTTCCTGGAGCTGCAGAGAGAGGGCGAGAAGCAGTGA
S0
SEQ ID NO: 19
ATGGCTAAAACAGCAATGGCCTACAAGGAAAAAATGAAGGAGCTGTCCATGCTGTCACTGATCTGCTCTTGCTT
TTACCCGGAACCTCGCAACATCAACATCTATACTTACGATGATATGGAAGTGAAGCAAATCAACAAACGTGCCT
CTGGCCAGGCTTTTGAGCTGATCTTGAAGCCACCATCTCCTATCTCAGAAGCCCCACGAACTTTAGCTTCTCCA
AAGAAGAAAGACCTGTCCCTGGAGGAGATCCAGAAGAAACTGGAGGCTGCAGAGGAAAGAAGAAAGTCTCAGGA
GGCCCAGGTGCTGAAACAATTGGCAGAGAAGAGGGAACACGAGCGAGAAGTCCTTCAGAAGGCTTTGGAGGAGA
ACAACAACTTCAGCAAGATGGCGGAGGAAAAGCTGATCCTGAAAATGGAACAAATTAAGGAAAACCGTGAGGCT
AATCTAGCTGCTATTATTGAACGTCTGCAGGAAAAGCTGGTCAAGTTTATTTCTTCTGAACTAAAAGAATCTAT
AGAGTCTCAATTTCTGGAGCTTCAGAGGGAAGGAGAGAAGCAATGA
WT protein sequence of Stathmin-2 (STMN2)
SEQ ID NO: 20
MAKTAMAYKEKMKELSMLSLICSCFYPEPRNINIYTYDDMEVKQINKRASGQAFELILKPPSPISEAPRTLASP
KKKDLSLEEIQKKLEAAEERRKSQEAQVLKOLAEKREHEREVLQKALEENNNFSKMAEEKLILKMEQIKENREA
NLAAIIERLQEKLVKFISSELKESIESQFLELQREGEKQ
MC1
SEQ ID NO: 21
ATGTGGACCCTCGTTTCTTGGGTCGCCCTGACAGCCGGGCTGGTTGCCGGCACTCGGTGCCCTGACGGACAGTT
TTGCCCCGTGGCTTGTTGTCTTGATCCAGGGGGGGCCAGTTACTCTTGTTGTAGGCCATTGCTTGACAAATGGC
CAACCACTCTTTCCAGGCACCTGGGAGGACCTTGTCAGGTGGACGCTCACTGCAGCGCTGGCCACAGCTGTATT
TTCACAGTGTCTGGGACATCATCCTGTTGTCCTTTCCCCGAAGCGGTGGCTTGTGGCGATGGCCATCATTGCTG
TCCTCGGGGATTTCATTGTAGCGCAGACGGACGCAGCTGCTTTCAACGGTCTGGAAACAATTCCGTCGGGGCCA
TACAATGCCCAGATTCTCAGTTTGAATGCCCCGATTTCTCTACCTGTTGCGTGATGGTGGACGGCTCCTGGGGC
TGCTGTCCTATGCCCCAGGCCTCCTGCTGCGAGGACAGGGTGCATTGTTGTCCTCATGGGGCCTTCTGTGACCT
CGTTCACACAAGGTGTATTACACCAACAGGCACTCACCCTCTGGCCAAGAAACTTCCTGCCCAGAGAACCAATA
GGGCAGTTGCTCTGAGCTCTTCAGTGATGTGCCCTGATGCCCGCTCTCGCTGTCCTGACGGCAGCACATGCTGT
GAACTCCCTTCAGGCAAGTATGGATGTTGTCCTATGCCCAATGCAACTTGCTGTTCCGACCACCTCCATTGCTG
TCCTCAAGATACAGTCTGTGACCTCATACAGTCAAAGTGCCTTTCCAAAGAAAACGCCACAACCGATCTTTTGA
CAAAGCTTCCCGCCCACACAGTTGGCGATGTGAAGTGTGATATGGAGGTGTCCTGCCCTGATGGATATACTTGC
TGCCGCCTGCAAAGTGGAGCATGGGGCTGCTGTCCATTCACCCAAGCCGTGTGCTGCGAGGATCATATCCACTG
TTGCCCTGCTGGGTTTACGTGCGATACTCAAAAAGGCACCTGCGAACAGGGACCACACCAAGTTCCCTGGATGG
AGAAAGCTCCAGCACATCTTTCTTTGCCGGATCCACAGGCCCTCAAGCGGGATGTCCCATGCGATAATGTGTCC
AGCTGCCCGAGTTCTGACACCTGCTGTCAGTTGACTTCTGGAGAGTGGGGATGTTGCCCAATTCCAGAGGCTGT
TTGCTGTTCAGATCATCAACACTGTTGCCCACAGGGTTATACCTGTGTCGCTGAAGGCCAATGTCAACGGGGCT
CTGAGATCGTTGCGGGCCTCGAGAAGATGCCCGCCCGGCGCGCCTCTTTGAGCCACCCAAGAGACATCGGATGC
GACCAGCACACCTCATGTCCTGTCGGACAGACTTGTTGTCCTTCCCTGGGTGGATCTTGGGCGTGTTGTCAACT
CCCCCATGCAGTATGCTGCGAAGATAGGCAACATTGCTGCCCAGCTGGGTATACCTGTAATGTCAAGGCTAGAT
CATGCGAAAAAGAGGTTGTTTCTGCTCAGCCAGCCACATTTCTCGCACGGAGCCCTCATGTGGGGGTTAAAGAC
GTCGAGTGTGGCGAAGGGCATTTCTGTCACGACAATCAAACATGTTGTCGGGATAACCGGCAGGGATGGGCATG
TTGTCCCTATCGGCAAGGCGTGTGCTGTGCCGACAGACGGCATTGCTGTCCAGCTGGGTTCAGATGTGCAGCAA
GAGGGACTAAATGTCTTCGGAGGGAGGCCCCCCGGTGGGATGCACCCCTTCGGGACCCCGCCCTGAGACAGCTG
CTGGCCACTAACTTCAGCCTTTTGAAGCAGGCCGGAGATGTCGAAGAAAACCCTGGACCTATGGCAAAGACTGC
TATGGCTTATAAGGAGAAAATGAAGGAACTCTCTATGCTCTCTCTCATTTGTTCCTGCTTCTATCCTGAGCCTC
GGAACATTAATATCTACACTTACGATGATATGGAGGTAAAGCAAATTAACAAGCGAGCGAGTGGGCAGGCCTTC
GAACTTATATTGAAGCCACCCTCCCCTATATCCGAAGCGCCGAGGACATTGGCCAGCCCAAAAAAGAAAGACCT
TTCCTTGGAGGAGATTCAAAAGAAGTTGGAGGCTGCGGAGGAAAGGAGAAAGTCTCAAGAGGCCCAAGTCTTGA
AGCAGCTCGCCGAAAAGAGGGAGCACGAAAGGGAGGTTCTCCAGAAAGCTCTCGAGGAAAACAATAACTTTTCA
AAAATGGCGGAGGAAAAACTCATCCTTAAGATGGAGCAAATTAAAGAAAATCGGGAGGCTAACCTCGCCGCAAT
AATCGAAAGGTTGCAAGAGAAGCTCGTCAAGTTTATTTCCTCAGAGCTTAAAGAATCAATAGAGTCCCAGTTTT
TGGAGCTCCAAAGAGAAGGCGAGAAGCAGTGA
MC2
SEQ ID NO: 22
ATGGCAAAGACAGCTATGGCATATAAGGAGAAAATGAAAGAACTGAGCATGCTGTCTCTGATATGTTCATGTTT
TTATCCTGAGCCACGCAATATTAACATTTATACATACGACGACATGGAGGTCAAACAAATAAATAAAAGAGCTT
CTGGACAAGCATTCGAACTCATACTCAAGCCACCCTCACCCATTTCTGAAGCCCCTAGGACTTTGGCATCACCA
AAAAAGAAAGACCTCAGCCTTGAAGAGATACAGAAGAAACTGGAAGCTGCAGAAGAGAGGCGCAAATCCCAGGA
GGCCCAAGTTCTGAAACAGCTCGCCGAGAAACGGGAACATGAAAGGGAAGTTCTCCAGAAGGCTCTGGAGGAGA
ACAACAATTTCTCTAAGATGGCTGAGGAGAAGTTGATCTTGAAGATGGAACAGATAAAGGAGAATAGAGAGGCG
AACCTGGCCGCCATCATAGAACGGCTGCAGGAAAAGCTGGTGAAGTTTATTAGCTCCGAACTCAAAGAAAGCAT
AGAATCCCAGTTTCTGGAACTGCAGCGGGAAGGTGAAAAACAGGCTACCAACTTTTCCTTGCTGAAGCAGGCAG
GAGATGTTGAAGAAAATCCTGGACCCATGTGGACACTCGTTAGTTGGGTTGCCTTGACCGCTGGATTGGTCGCC
GGAACACGCTGTCCTGATGGACAGTTTTGTCCAGTCGCATGCTGTTTGGATCCTGGGGGCGCTAGTTACAGCTG
CTGCAGGCCACTGCTCGATAAATGGCCGACTACATTGTCACGCCATTTGGGAGGGCCTTGCCAAGTCGATGCTC
ACTGTTCCGCCGGTCATTCTTGCATTTTCACAGTTTCCGGCACTAGCTCATGCTGCCCGTTTCCGGAAGCCGTT
GCCTGTGGCGATGGGCATCATTGCTGTCCTCGGGGCTTTCACTGCAGCGCCGACGGAAGGTCCTGCTTCCAGAG
GTCAGGGAACAATAGCGTTGGAGCGATTCAATGCCCTGACTCTCAATTTGAGTGTCCAGATTTTAGCACCTGCT
GTGTCATGGTCGATGGATCTTGGGGCTGTTGCCCCATGCCTCAAGCGTCATGTTGCGAGGATCGCGTCCACTGT
TGTCCACACGGAGCCTTTTGTGACCTGGTACATACTCGGTGCATCACACCAACCGGGACGCACCCTTTGGCCAA
AAAGCTTCCCGCTCAGAGAACAAATCGGGCCGTCGCACTCAGTAGTTCCGTAATGTGTCCCGACGCTCGCTCTC
GGTGCCCAGACGGCTCTACATGCTGCGAGCTGCCGTCAGGGAAGTATGGCTGCTGTCCTATGCCCAACGCTACT
TGTTGTTCAGACCACCTCCATTGCTGTCCTCAGGATACCGTTTGTGATCTCATTCAAAGTAAGTGTCTGTCCAA
AGAAAATGCTACTACCGACTTGCTGACTAAACTTCCAGCTCATACAGTGGGGGATGTGAAATGCGACATGGAAG
TCTCTTGTCCAGACGGGTATACATGCTGTAGGCTGCAGTCAGGGGCCTGGGGATGCTGTCCTTTTACTCAAGCT
GTTTGTTGTGAAGACCATATACACTGCTGCCCCGCAGGCTTTACCTGTGATACCCAGAAAGGAACATGTGAGCA
GGGTCCACATCAAGTGCCTTGGATGGAGAAAGCCCCCGCTCACCTTTCTCTTCCTGACCCGCAGGCGTTGAAGA
GGGACGTGCCTTGTGACAATGTTTCATCATGTCCAAGCTCTGACACCTGTTGTCAACTGACATCTGGCGAGTGG
GGATGTTGCCCAATCCCAGAAGCCGTCTGTTGTTCTGATCACCAACACTGTTGCCCACAGGGCTATACCTGCGT
GGCCGAAGGTCAATGCCAAAGAGGGAGCGAGATTGTTGCGGGACTGGAAAAAATGCCCGCTCGGAGGGCCAGCC
TGTCCCACCCACGAGACATAGGATGTGACCAGCATACCTCTTGTCCGGTTGGTCAGACGTGCTGCCCAAGCTTG
GGGGGATCTTGGGCATGTTGTCAACTGCCGCACGCAGTTTGTTGCGAAGACAGACAACACTGCTGTCCCGCGGG
CTACACTTGTAACGTTAAGGCAAGAAGCTGCGAAAAAGAGGTGGTTTCTGCCCAGCCAGCAACCTTCCTCGCTA
GATCCCCTCACGTTGGCGTGAAGGACGTGGAGTGCGGCGAGGGACATTTCTGTCACGATAACCAAACCTGCTGT
CGCGACAACCGCCAGGGTTGGGCCTGCTGCCCTTATCGCCAGGGAGTGTGCTGTGCCGATCGGCGCCATTGTTG
TCCTGCAGGGTTTAGATGCGCTGCTAGGGGTACTAAATGTCTGAGAAGGGAAGCTCCGAGATGGGATGCCCCTT
TGAGAGATCCAGCTCTGAGACAATTGCTCTGA
MC3
SEQ ID NO: 23
ATGTGGACACTTGTTTCATGGGTTGCACTCACCGCTGGACTGGTCGCTGGAACCAGATGTCCAGACGGACAGTT
TTGCCCAGTGGCATGCTGTCTCGATCCTGGCGGAGCTAGCTACTCTTGCTGCAGACCACTGTTGGATAAGTGGC
CAACAACACTGAGCAGACATCTGGGCGGCCCATGCCAAGTGGATGCTCATTGTAGCGCCGGCCACTCCTGCATT
TTCACTGTGTCCGGTACCTCCTCATGCTGCCCATTCCCTGAAGCCGTCGCATGCGGAGATGGGCATCACTGTTG
CCCCAGAGGTTTTCACTGCAGCGCTGATGGGCGGTCTTGCTTCCAAAGGTCCGGAAACAATAGCGTTGGGGCGA
TTCAGTGCCCTGACTCCCAATTTGAATGCCCTGACTTTTCAACCTGTTGTGTAATGGTCGATGGCAGCTGGGGC
TGCTGCCCCATGCCACAGGCTTCTTGTTGCGAAGACCGCGTGCATTGTTGCCCTCATGGAGCGTTTTGTGACCT
CGTGCACACGAGGTGCATTACTCCCACAGGCACTCATCCACTGGCTAAGAAATTGCCTGCCCAGAGAACAAACA
GGGCAGTAGCCTTGAGCTCTTCAGTAATGTGTCCGGACGCCAGATCAAGGTGCCCCGATGGATCTACTTGCTGC
GAGCTCCCCTCCGGCAAGTACGGATGTTGTCCAATGCCAAATGCTACTTGCTGTAGTGACCACCTGCACTGTTG
TCCACAGGATACGGTCTGTGACTTGATACAGTCTAAGTGCCTGTCTAAGGAAAATGCAACAACCGATTTGCTTA
CAAAACTGCCAGCCCATACCGTAGGAGATGTCAAGTGCGACATGGAAGTTAGTTGTCCCGACGGCTATACGTGT
TGTCGACTGCAATCCGGAGCCTGGGGTTGTTGCCCGTTTACTCAGGCTGTATGTTGCGAGGACCACATCCACTG
TTGTCCCGCGGGGTTCACCTGTGACACCCAGAAAGGTACCTGCGAGCAGGGGCCTCATCAGGTACCGTGGATGG
AAAAAGCTCCTGCACACTTGTCACTGCCGGATCCTCAGGCGCTCAAGCGGGATGTCCCTTGCGACAATGTAAGC
TCCTGTCCGTCTAGTGATACGTGTTGCCAACTTACCTCCGGGGAATGGGGCTGTTGCCCTATACCAGAAGCTGT
CTGTTGCTCTGATCATCAACATTGCTGCCCCCAGGGTTACACATGTGTAGCAGAGGGTCAGTGTCAAAGAGGGT
CAGAAATTGTAGCTGGCTTGGAAAAAATGCCCGCAAGGCGCGCATCCCTGTCTCACCCTCGGGATATAGGATGC
GATCAGCACACCTCTTGTCCAGTGGGTCAGACTTGTTGTCCCAGCTTGGGGGGCAGCTGGGCTTGTTGTCAATT
GCCTCACGCAGTTTGCTGTGAAGACCGCCAACATTGCTGTCCTGCTGGATACACATGCAATGTCAAGGCTAGGA
GCTGTGAAAAAGAGGTGGTGTCTGCACAGCCCGCAACCTTTCTTGCACGCTCTCCTCATGTCGGAGTGAAAGAT
GTTGAATGCGGAGAAGGACACTTTTGCCATGACAATCAGACTTGTTGCAGGGACAACAGACAAGGATGGGCTTG
TTGCCCTTATCGCCAAGGAGTATGCTGTGCGGATAGAAGACATTGTTGCCCGGCCGGGTTTAGGTGTGCTGCTA
GAGGCACCAAATGTCTGAGGCGAGAAGCACCTAGATGGGACGCTCCACTGCGGGACCCTGCCTTGAGACAACTC
CTGGCTACTAACTTCAGCCTGCTGAAGCAGGCTGGAGACGTGGAGGAGAACCCTGGACCTATGGCTAAGACAGC
AATGGCTTATAAAGAGAAGATGAAAGAGCTCAGCATGTTGTCCTTGATTTGCTCTTGTTTCTACCCAGAACCAC
GGAACATAAACATATATACATATGATGATATGGAAGTGAAGCAAATCAATAAAAGGGCCTCTGGTCAAGCGTTT
GAACTGATCTTGAAGCCTCCCTCCCCAATTAGCGAGGCCCCAAGGACCCTCGCTTCTCCCAAAAAAAAGGACCT
CTCTCTGGAGGAGATCCAAAAGAAGTTGGAGGCAGCCGAGGAAAGGCGCAAATCCCAGGAAGCGCAAGTCCTGA
AGCAGCTCGCCGAAAAGAGGGAGCACGAGCGGGAGGTCCTCCAAAAAGCACTCGAAGAAAATAATAACTTTTCT
AAGATGGCTGAGGAAAAATTGATACTCAAGATGGAGCAGATTAAAGAAAATAGAGAAGCTAACCTGGCAGCCAT
TATAGAGAGGCTCCAGGAGAAGTTGGTGAAGTTTATCAGCTCTGAACTGAAAGAGTCAATCGAATCTCAATTTC
TTGAGCTCCAGAGGGAAGGAGAAAAGCAATGA
MC4
SEQ ID NO: 24
ATGGCTAAGACAGCAATGGCTTATAAAGAGAAGATGAAAGAGCTCAGCATGTTGTCCTTGATTTGCTCTTGTTT
CTACCCAGAACCACGGAACATAAACATATATACATATGATGATATGGAAGTGAAGCAAATCAATAAAAGGGCCT
CTGGTCAAGCGTTTGAACTGATCTTGAAGCCTCCCTCCCCAATTAGCGAGGCCCCAAGGACCCTCGCTTCTCCC
AAAAAAAAGGACCTCTCTCTGGAGGAGATCCAAAAGAAGTTGGAGGCAGCCGAGGAAAGGCGCAAATCCCAGGA
AGCGCAAGTCCTGAAGCAGCTCGCCGAAAAGAGGGAGCACGAGCGGGAGGTCCTCCAAAAAGCACTCGAAGAAA
ATAATAACTTTTCTAAGATGGCTGAGGAAAAATTGATACTCAAGATGGAGCAGATTAAAGAAAATAGAGAAGCT
AACCTGGCAGCCATTATAGAGAGGCTCCAGGAGAAGTTGGTGAAGTTTATCAGCTCTGAACTGAAAGAGTCAAT
CGAATCTCAATTTCTTGAGCTCCAGAGGGAAGGAGAAAAGCAAGCTACTAACTTCAGCCTGCTGAAGCAGGCTG
GAGACGTGGAGGAGAACCCTGGACCTATGTGGACACTTGTTTCATGGGTTGCACTCACCGCTGGACTGGTCGCT
GGAACCAGATGTCCAGACGGACAGTTTTGCCCAGTGGCATGCTGTCTCGATCCTGGCGGAGCTAGCTACTCTTG
CTGCAGACCACTGTTGGATAAGTGGCCAACAACACTGAGCAGACATCTGGGCGGCCCATGCCAAGTGGATGCTC
ATTGTAGCGCCGGCCACTCCTGCATTTTCACTGTGTCCGGTACCTCCTCATGCTGCCCATTCCCTGAAGCCGTC
GCATGCGGAGATGGGCATCACTGTTGCCCCAGAGGTTTTCACTGCAGCGCTGATGGGCGGTCTTGCTTCCAAAG
GTCCGGAAACAATAGCGTTGGGGCGATTCAGTGCCCTGACTCCCAATTTGAATGCCCTGACTTTTCAACCTGTT
GTGTAATGGTCGATGGCAGCTGGGGCTGCTGCCCCATGCCACAGGCTTCTTGTTGCGAAGACCGCGTGCATTGT
TGCCCTCATGGAGCGTTTTGTGACCTCGTGCACACGAGGTGCATTACTCCCACAGGCACTCATCCACTGGCTAA
GAAATTGCCTGCCCAGAGAACAAACAGGGCAGTAGCCTTGAGCTCTTCAGTAATGTGTCCGGACGCCAGATCAA
GGTGCCCCGATGGATCTACTTGCTGCGAGCTCCCCTCCGGCAAGTACGGATGTTGTCCAATGCCAAATGCTACT
TGCTGTAGTGACCACCTGCACTGTTGTCCACAGGATACGGTCTGTGACTTGATACAGTCTAAGTGCCTGTCTAA
GGAAAATGCAACAACCGATTTGCTTACAAAACTGCCAGCCCATACCGTAGGAGATGTCAAGTGCGACATGGAAG
TTAGTTGTCCCGACGGCTATACGTGTTGTCGACTGCAATCCGGAGCCTGGGGTTGTTGCCCGTTTACTCAGGCT
GTATGTTGCGAGGACCACATCCACTGTTGTCCCGCGGGGTTCACCTGTGACACCCAGAAAGGTACCTGCGAGCA
GGGGCCTCATCAGGTACCGTGGATGGAAAAAGCTCCTGCACACTTGTCACTGCCGGATCCTCAGGCGCTCAAGC
GGGATGTCCCTTGCGACAATGTAAGCTCCTGTCCGTCTAGTGATACGTGTTGCCAACTTACCTCCGGGGAATGG
GGCTGTTGCCCTATACCAGAAGCTGTCTGTTGCTCTGATCATCAACATTGCTGCCCCCAGGGTTACACATGTGT
AGCAGAGGGTCAGTGTCAAAGAGGGTCAGAAATTGTAGCTGGCTTGGAAAAAATGCCCGCAAGGCGCGCATCCC
TGTCTCACCCTCGGGATATAGGATGCGATCAGCACACCTCTTGTCCAGTGGGTCAGACTTGTTGTCCCAGCTTG
GGGGGCAGCTGGGCTTGTTGTCAATTGCCTCACGCAGTTTGCTGTGAAGACCGCCAACATTGCTGTCCTGCTGG
ATACACATGCAATGTCAAGGCTAGGAGCTGTGAAAAAGAGGTGGTGTCTGCACAGCCCGCAACCTTTCTTGCAC
GCTCTCCTCATGTCGGAGTGAAAGATGTTGAATGCGGAGAAGGACACTTTTGCCATGACAATCAGACTTGTTGC
AGGGACAACAGACAAGGATGGGCTTGTTGCCCTTATCGCCAAGGAGTATGCTGTGCGGATAGAAGACATTGTTG
CCCGGCCGGGTTTAGGTGTGCTGCTAGAGGCACCAAATGTCTGAGGCGAGAAGCACCTAGATGGGACGCTCCAC
TGCGGGACCCTGCCTTGAGACAACTCCTGTGA
MC5
SEQ ID NO: 25
ATGTGGACCCTCGTGTCCTGGGTGGCACTGACAGCCGGGTTGGTGGCTGGAACTAGATGCCCAGACGGACAATT
CTGTCCCGTGGCTTGTTGCTTGGACCCTGGAGGCGCATCCTATAGCTGTTGCAGGCCCTTGCTGGACAAATGGC
CAACCACACTGTCTCGGCATTTGGGCGGCCCATGCCAGGTGGATGCTCATTGCAGCGCCGGCCACAGCTGCATC
TTCACCGTCTCCGGAACAAGCAGCTGTTGTCCATTTCCAGAAGCGGTCGCTTGTGGCGATGGACATCACTGTTG
TCCTCGCGGATTTCACTGCTCTGCCGATGGAAGGTCCTGCTTTCAACGGAGCGGAAATAATTCTGTCGGGGCCA
TTCAATGTCCCGACTCCCAGTTCGAGTGCCCTGATTTTAGCACATGTTGTGTCATGGTCGATGGCAGCTGGGGA
TGCTGCCCAATGCCACAGGCATCCTGCTGCGAAGATAGGGTACATTGTTGCCCTCATGGAGCCTTTTGTGATCT
CGTTCATACAAGGTGTATTACTCCAACTGGCACACATCCCCTCGCCAAGAAATTGCCTGCACAGAGAACCAACC
GGGCTGTGGCCCTCTCTTCCTCTGTTATGTGTCCAGACGCACGGTCTAGATGTCCCGATGGATCTACATGTTGC
GAACTGCCTAGCGGAAAGTATGGGTGCTGTCCCATGCCTAACGCTACGTGCTGCTCTGATCATTTGCACTGCTG
TCCACAGGATACAGTGTGTGATCTGATTCAAAGCAAATGCCTGTCCAAAGAAAATGCAACAACCGATCTCTTGA
CTAAACTCCCTGCCCACACCGTGGGCGACGTAAAGTGTGACATGGAGGTCTCTTGCCCAGACGGCTATACATGC
TGTCGGCTGCAGTCTGGAGCCTGGGGCTGTTGCCCCTTCACTCAGGCCGTCTGTTGCGAGGATCATATCCATTG
TTGTCCAGCAGGATTTACTTGCGACACCCAGAAAGGCACCTGCGAGCAAGGCCCACACCAAGTGCCTTGGATGG
AGAAGGCCCCAGCTCACCTCAGCCTGCCTGACCCTCAGGCACTCAAGAGGGATGTGCCATGTGACAATGTTTCT
TCCTGCCCTTCCAGCGATACATGTTGTCAATTGACATCTGGCGAATGGGGATGTTGTCCCATCCCAGAGGCTGT
GTGCTGCTCCGATCACCAGCACTGTTGTCCACAGGGGTATACATGTGTTGCTGAAGGCCAATGTCAACGGGGCT
CCGAAATCGTGGCAGGACTCGAAAAAATGCCTGCAAGGAGAGCTAGCCTCTCTCATCCACGCGATATCGGATGC
GACCAACATACATCCTGCCCAGTCGGCCAAACTTGCTGTCCCTCACTTGGAGGTTCCTGGGCTTGCTGCCAGCT
GCCCCATGCCGTGTGCTGCGAAGATAGACAGCACTGTTGCCCTGCCGGGTACACCTGCAATGTCAAGGCCAGAT
CATGCGAAAAAGAGGTGGTCAGCGCACAGCCAGCTACATTCTTGGCCAGATCCCCACATGTGGGCGTGAAAGAC
GTCGAATGCGGCGAAGGCCATTTCTGCCACGACAACCAAACCTGCTGCAGGGACAACAGGCAGGGATGGGCTTG
CTGTCCATATAGACAAGGGGTGTGCTGCGCAGACAGGAGACATTGCTGCCCTGCTGGGTTCCGGTGCGCTGCAA
GGGGAACTAAGTGCCTGAGACGGGAGGCACCTCGGTGGGATGCCCCACTGAGGGACCCCGCTTTGAGACAGCTC
CTGGCTACTAACTTCAGCCTGCTGAAGCAGGCTGGAGACGTGGAGGAGAACCCTGGACCTATGGCCAAGACCGC
TATGGCCTATAAGGAAAAGATGAAGGAGCTCTCCATGCTGAGCCTCATTTGTAGCTGCTTCTACCCTGAGCCCC
GGAACATTAACATCTACACCTATGACGACATGGAAGTCAAACAGATAAATAAGCGGGCATCCGGCCAGGCCTTC
GAACTGATCCTCAAGCCACCTTCTCCTATTTCCGAAGCTCCCAGGACCTTGGCCTCCCCCAAGAAAAAGGATCT
GAGCCTCGAAGAGATTCAGAAGAAGCTGGAAGCTGCTGAGGAGCGGAGGAAGAGCCAGGAGGCGCAGGTGCTGA
AGCAGCTCGCCGAGAAGAGGGAGCATGAACGGGAGGTCTTGCAGAAGGCCCTCGAAGAGAACAATAACTTCTCC
AAGATGGCTGAGGAAAAGCTCATCCTCAAGATGGAGCAAATCAAAGAGAACAGAGAGGCCAACCTGGCTGCAAT
CATCGAGAGGCTCCAGGAAAAGCTCGTGAAATTCATTTCTAGCGAGCTCAAAGAATCTATCGAGTCCCAGTTTT
TGGAACTCCAGAGGGAAGGCGAAAAACAATGA
MC6
SEQ ID NO: 26
ATGGCCAAGACCGCTATGGCCTATAAGGAAAAGATGAAGGAGCTCTCCATGCTGAGCCTCATTTGTAGCTGCTT
CTACCCTGAGCCCCGGAACATTAACATCTACACCTATGACGACATGGAAGTCAAACAGATAAATAAGCGGGCAT
CCGGCCAGGCCTTCGAACTGATCCTCAAGCCACCTTCTCCTATTTCCGAAGCTCCCAGGACCTTGGCCTCCCCC
AAGAAAAAGGATCTGAGCCTCGAAGAGATTCAGAAGAAGCTGGAAGCTGCTGAGGAGCGGAGGAAGAGCCAGGA
GGCGCAGGTGCTGAAGCAGCTCGCCGAGAAGAGGGAGCATGAACGGGAGGTCTTGCAGAAGGCCCTCGAAGAGA
ACAATAACTTCTCCAAGATGGCTGAGGAAAAGCTCATCCTCAAGATGGAGCAAATCAAAGAGAACAGAGAGGCC
AACCTGGCTGCAATCATCGAGAGGCTCCAGGAAAAGCTCGTGAAATTCATTTCTAGCGAGCTCAAAGAATCTAT
CGAGTCCCAGTTTTTGGAACTCCAGAGGGAAGGCGAAAAACAAGCTACTAACTTCAGCCTGCTGAAGCAGGCTG
GAGACGTGGAGGAGAACCCTGGACCTATGTGGACCCTCGTGTCCTGGGTGGCACTGACAGCCGGGTTGGTGGCT
GGAACTAGATGCCCAGACGGACAATTCTGTCCCGTGGCTTGTTGCTTGGACCCTGGAGGCGCATCCTATAGCTG
TTGCAGGCCCTTGCTGGACAAATGGCCAACCACACTGTCTCGGCATTTGGGCGGCCCATGCCAGGTGGATGCTC
ATTGCAGCGCCGGCCACAGCTGCATCTTCACCGTCTCCGGAACAAGCAGCTGTTGTCCATTTCCAGAAGCGGTC
GCTTGTGGCGATGGACATCACTGTTGTCCTCGCGGATTTCACTGCTCTGCCGATGGAAGGTCCTGCTTTCAACG
GAGCGGAAATAATTCTGTCGGGGCCATTCAATGTCCCGACTCCCAGTTCGAGTGCCCTGATTTTAGCACATGTT
GTGTCATGGTCGATGGCAGCTGGGGATGCTGCCCAATGCCACAGGCATCCTGCTGCGAAGATAGGGTACATTGT
TGCCCTCATGGAGCCTTTTGTGATCTCGTTCATACAAGGTGTATTACTCCAACTGGCACACATCCCCTCGCCAA
GAAATTGCCTGCACAGAGAACCAACCGGGCTGTGGCCCTCTCTTCCTCTGTTATGTGTCCAGACGCACGGTCTA
GATGTCCCGATGGATCTACATGTTGCGAACTGCCTAGCGGAAAGTATGGGTGCTGTCCCATGCCTAACGCTACG
TGCTGCTCTGATCATTTGCACTGCTGTCCACAGGATACAGTGTGTGATCTGATTCAAAGCAAATGCCTGTCCAA
AGAAAATGCAACAACCGATCTCTTGACTAAACTCCCTGCCCACACCGTGGGCGACGTAAAGTGTGACATGGAGG
TCTCTTGCCCAGACGGCTATACATGCTGTCGGCTGCAGTCTGGAGCCTGGGGCTGTTGCCCCTTCACTCAGGCC
GTCTGTTGCGAGGATCATATCCATTGTTGTCCAGCAGGATTTACTTGCGACACCCAGAAAGGCACCTGCGAGCA
AGGCCCACACCAAGTGCCTTGGATGGAGAAGGCCCCAGCTCACCTCAGCCTGCCTGACCCTCAGGCACTCAAGA
GGGATGTGCCATGTGACAATGTTTCTTCCTGCCCTTCCAGCGATACATGTTGTCAATTGACATCTGGCGAATGG
GGATGTTGTCCCATCCCAGAGGCTGTGTGCTGCTCCGATCACCAGCACTGTTGTCCACAGGGGTATACATGTGT
TGCTGAAGGCCAATGTCAACGGGGCTCCGAAATCGTGGCAGGACTCGAAAAAATGCCTGCAAGGAGAGCTAGCC
TCTCTCATCCACGCGATATCGGATGCGACCAACATACATCCTGCCCAGTCGGCCAAACTTGCTGTCCCTCACTT
GGAGGTTCCTGGGCTTGCTGCCAGCTGCCCCATGCCGTGTGCTGCGAAGATAGACAGCACTGTTGCCCTGCCGG
GTACACCTGCAATGTCAAGGCCAGATCATGCGAAAAAGAGGTGGTCAGCGCACAGCCAGCTACATTCTTGGCCA
GATCCCCACATGTGGGCGTGAAAGACGTCGAATGCGGCGAAGGCCATTTCTGCCACGACAACCAAACCTGCTGC
AGGGACAACAGGCAGGGATGGGCTTGCTGTCCATATAGACAAGGGGTGTGCTGCGCAGACAGGAGACATTGCTG
CCCTGCTGGGTTCCGGTGCGCTGCAAGGGGAACTAAGTGCCTGAGACGGGAGGCACCTCGGTGGGATGCCCCAC
TGAGGGACCCCGCTTTGAGACAGCTCCTGTGA
EFShI1
SEQ ID NO: 27
GGGCAGAGCGCACATCGCCCACAGTCCCCGAGAAGTTGGGGGGAGGGGTCGGCAATTGAACCGGTGCCTAGAGA
AGGTGGCGCGGGGTAAACTGGGAAAGTGATGTCGTGTACTGGCTCCGCCTTTTTCCCGAGGGTGGGGGAGAACC
GTATATAAGTGCAGTAGTCGCCGTGAACGTTCTTTTTCGCAACGGGTTTGCCGCCAGAACACAGGGAGTCGCTG
CGACGCTGCCTTCGCCCCGTGCCCCGCTCCGCCGCCGCCTCGCGCCGCCCGCCCCGGCTCTGACTGACCGCGTT
ACTCCCACAGGTGAGCGGGCGGGACGGCCCTTCTCCTCCGGGCTGTAATTAGCTGAGCAAGAGGTAAGGGTTTA
AGGGATGGTTGGTTGGTGGGGTATTAATGTTTAATTACCTGGAGCACCTGCCTGAAATCACTTTTTTTCAG
SV40 polyA
SEQ ID NO: 28
AACTTGTTTATTGCAGCTTATAATGGTTACAAATAAAGCAATAGCATCACAAATTTCACAAATAAAGCATTTTT
TTCACTGCATTCTAGTTGTGGTTTGTCCAAACTCATCAATGTATCTTA
hGH polyA
SEQ ID NO: 29
GGGTGGCATCCCTGTGACCCCTCCCCAGTGCCTCTCCTGGCCCTGGAAGTTGCCACTCCAGTGCCCACCAGCCT
TGTCCTAATAAAATTAAGTTGCATCATTTTGTCTGACTAGGTGTCCTTCTATAATATTATGGGGTGGAGGGGGG
TGGTATGGAGCAAGGGGCAAGTTGGGAAGACAACCTGTAGGGCCTGCGGGGTCTATTGGGAACCAAGCTGGAGT
GCAGTGGCACAATCTTGGCTCACTGCAATCTCCGCCTCCTGGGTTCAAGCGATTCTCCTGCCTCAGCCTCCCGA
GTTGTTGGGATTCCAGGCATGCATGACCAGGCTCAGCTAATTTTTGTTTTTTTGGTAGAGACGGGGTTTCACCA
TATTGGCCAGGCTGGTCTCCAACTCCTAATCTCAGGTGATCTACCCACCTTGGCCTCCCAAATTGCTGGGATTA
CAGGCGTGAACCACTGCTCCCTTCCCTGTCCTT
WPRE
SEQ ID NO: 30
AATCAACCTCTGGATTACAAAATTTGTGAAAGATTGACTGGTATTCTTAACTATGTTGCTCCTTTTACCCTATG
TGGATAGGCTGCTTTAATGCCTTTGTATCATGCTATTGCTTCCAGTATGGCTTTCATTTTCTCCTCCTTGTATA
AATCCTGGTTGCTGTCTCTTTATGAGGAGTTGTGGCCAGTTGTCAGGCAACCTGGCCTGGTGTGCACTGTGTTT
GCTGAGGCAACCCCCACTGGTTGGGGCATTGCCACCACCTGTCAGCTCCTTTCCTGGACTTTCCCTTTCCCCCT
CCCTATTGCCAGGGCAGAACTCATCACTGCCTGCCTTGCCTGCTGCTGGACAGGGGCTGGGCTGTTGGGCACTG
ACAATTCCCTGGTGTTGTCAGGGAAATCATGGTCCTTTCCTTGGCTGCTGGCCTGTGTTGCCACCTGGATTCTG
GGCAGGAGGTCCTTCTGCTAGGTCCCTTGGGCCCTCAATCCAGAGGACCTTCCTTCCAGCAGCCTGCTGCAGGC
TCTGCAGCCTCTTCAGCTTCTTGGCCTTGGCCCTCAGAGGAGTGGGATCTCCCTTTGGGCCTCCTCCCTGC
P2A-1 (in MC1)
SEQ ID NO: 31
GCCACTAACTTCAGCCTTTTGAAGCAGGCCGGAGATGTCGAAGAAAACCCTGGACCT
P2A-2 (in MC2)
SEQ ID NO: 32
GCTACCAACTTTTCCTTGCTGAAGCAGGCAGGAGATGTTGAAGAAAATCCTGGACCC
P2A-3 (in MC3, MC4, MC5, MC6)
SEQ ID NO: 33
GCTACTAACTTCAGCCTGCTGAAGCAGGCTGGAGACGTGGAGGAGAACCCTGGACCT
Wild-type AAV9 VP1 polypeptide sequence (Q588 and A589 are underlined)
SEQ ID NO: 34
MAADGYLPDWLEDNLSEGIREWWALKPGAPQPKANQQHQDNARGLVLPGYKYLGPGNGLDKGEPVNAADAAALE
HDKAYDQQLKAGDNPYLKYNHADAEFQERLKEDTSFGGNLGRAVFQAKKRLLEPLGLVEEAAKTAPGKKRPVEQ
SPQEPDSSAGIGKSGAQPAKKRLNFGQTGDTESVPDPQPIGEPPAAPSGVGSLTMASGGGAPVADNNEGADGVG
SSSGNWHCDSQWLGDRVITTSTRTWALPTYNNHLYKQISNSTSGGSSNDNAYFGYSTPWGYFDFNRFHCHFSPR
DWQRLINNNWGFRPKRLNFKLFNIQVKEVTDNNGVKTIANNLTSTVQVFTDSDYQLPYVLGSAHEGCLPPFPAD
VFMIPQYGYLTLNDGSQAVGRSSFYCLEYFPSQMLRTGNNFQFSYEFENVPFHSSYAHSQSLDRLMNPLIDQYL
YYLSKTINGSGQNQQTLKFSVAGPSNMAVQGRNYIPGPSYRQQRVSTTVTQNNNSEFAWPGASSWALNGRNSLM
NPGPAMASHKEGEDRFFPLSGSLIFGKQGTGRDNVDADKVMITNEEEIKTTNPVATESYGQVATNHQSAQAQAQ
TGWVQNQGILPGMVWQDRDVYLQGPIWAKIPHTDGNFHPSPLMGGFGMKHPPPQILIKNTPVPADPPTAFNKDK
LNSFITQYSTGQVSVEIEWELQKENSKRWNPEIQYTSNYYKSNNVEFAVNTEGVYSEPRPIGTRYLTRNL
ViVec-N001
SEQ ID NO: 35
GVFVLPN
ViVec-N002
SEQ ID NO: 36
GNYRGNP
ViVec-N003
SEQ ID NO: 37
CSSRRSK
ViVec-N004
SEQ ID NO: 38
FRHGPPS
ViVec-N005
SEQ ID NO: 39
RFKTGYP
ViVec-N006
SEQ ID NO: 40
GKHPAKL
ViVec-N007
SEQ ID NO: 41
NRGRSGE
ViVec-N008
SEQ ID NO: 42
REPRVGP
ViVec-N009
SEQ ID NO: 43
VTFSHAQ
ViVec-N010
SEQ ID NO: 44
DSRLTGR
ViVec-N011
SEQ ID NO: 45
EPIARPL
ViVec-N012
SEQ ID NO: 46
SSSWRPK
ViVec-N013
SEQ ID NO: 47
DLRDVLG
ViVec-N014
SEQ ID NO: 48
PYRSALW
ViVec-N015
SEQ ID NO: 49
LKPYHLE
ViVec-N016
SEQ ID NO: 50
GNKPNVD
ViVec-N017
SEQ ID NO: 51
WTAVLVQ
ViVec-N018
SEQ ID NO: 52
HTESTYG
ViVec-N019
SEQ ID NO: 53
PDEKMTK
ViVec-N020
SEQ ID NO: 54
QPWQQWQ
ViVec-N021
SEQ ID NO: 55
LMLRPYM
ViVec-N022
SEQ ID NO: 56
WRNQQVG
ViVec-N023
SEQ ID NO: 57
VGRLARA
ViVec-N024
SEQ ID NO: 58
LRRSRMS
ViVec-N025
SEQ ID NO: 59
ILHAARA
ViVec-N026
SEQ ID NO: 60
AGHTTKV
ViVec-N027
SEQ ID NO: 61
AGDEWRP
ViVec-N028
SEQ ID NO: 62
VTKKQET
ViVec-N029
SEQ ID NO: 63
RPMSTCS
ViVec-N030
SEQ ID NO: 64
RQRRANK
ViVec-N031
SEQ ID NO: 65
HLETARQ
ViVec-N032
SEQ ID NO: 66
LSDAKMG
ViVec-N033
SEQ ID NO: 67
TRITSVY
ViVec-N034
SEQ ID NO: 68
LWIESRP
ViVec-N035
SEQ ID NO: 69
AKGDMCN
ViVec-N036
SEQ ID NO: 70
VPDLKDC
ViVec-N037
SEQ ID NO: 71
VQSESHG
ViVec-N038
SEQ ID NO: 72
FFLDRPR
ViVec-N039
SEQ ID NO: 73
VLTATID
ViVec-N040
SEQ ID NO: 74
KGGAMCC
ViVec-N041
SEQ ID NO: 75
IWHVRYE
ViVec-N042
SEQ ID NO: 76
TGEHATT
ViVec-N043
SEQ ID NO: 77
DKSTQPC
ViVec-N044
SEQ ID NO: 78
PAVVIAN
ViVec-N045
SEQ ID NO: 79
VTGDYGM
ViVec-N046
SEQ ID NO: 80
EWSSKKT
ViVec-N047
SEQ ID NO: 81
NRGVSIE
ViVec-N048
SEQ ID NO: 82
PTDRQWP
ViVec-N049
SEQ ID NO: 83
ISASYAR
ViVec-N050
SEQ ID NO: 84
GPCSLPG
ViVec-N051
SEQ ID NO: 85
DYAAPCQ
ViVec-N052
SEQ ID NO: 86
GTREVEW
ViVec-N053
SEQ ID NO: 87
RNVSPAR
ViVec-N054
SEQ ID NO: 88
TGLGWTG
ViVec-N055
SEQ ID NO: 89
SFERTDK
ViVec-N056
SEQ ID NO: 90
QLDGDRS
ViVec-N057
SEQ ID NO: 91
WWEPTTT
ViVec-N058
SEQ ID NO: 92
DILTNYR
ViVec-N059
SEQ ID NO: 93
HSYDRTS
ViVec-N060
SEQ ID NO: 94
LPSAQLM

What is claimed is:

1. An isolated nucleic acid molecule, comprising a first polynucleotide sequence encoding a first polypeptide and a second polynucleotide sequence encoding a second polypeptide, wherein the first polypeptide is progranulin (PGRN) and the second polypeptide is stathmin-2 (STMN2); or the first polypeptide is stathmin-2 (STMN2) and the second polypeptide is progranulin (PGRN), wherein the first polynucleotide sequence is located at 5′ upstream of the second nucleotide sequence, wherein the polypeptide of progranulin (PGRN) comprises or consists of a polypeptide sequence of SEQ ID NO: 10, or a variant, homolog or orthohomolog thereof, and/or the polypeptide of stathmin-2 (STMN2) comprises or consists of a polypeptide sequence of SEQ ID NO: 20, or a variant, homolog or orthohomolog thereof.

2. The isolated nucleic acid molecule of claim 1, wherein the polynucleotide sequence encoding PGRN is a polynucleotide sequence selected from a group consisting of a polynucleotide sequence as shown in any one of SEQ ID NOs: 1-9, and/or the polynucleotide sequence encoding STMN2 is a polynucleotide sequence selected from a group consisting of a polynucleotide sequence as shown in any one of SEQ ID NOs: 11-19.

3. The isolated nucleic acid molecule of claim 1, wherein the first polynucleotide sequence and the second polynucleotide sequence are linked in frame and are operatively linked to a single promoter located at the 5′ upstream of both the first and the second nucleotide sequences.

4. The isolated nucleic acid molecule of claim 3, wherein the promoter is an EF1α promoter, an EFS promoter, or a variant or a derivative thereof.

5. The isolated nucleic acid molecule of claim 3, wherein the promoter is EFShI1 (SEQ ID NO: 27).

6. The isolated nucleic acid molecule of claim 1, wherein the isolated nucleic acid molecule further comprises a linker sequence between the first and the second polynucleotide sequences.

7. The isolated nucleic acid molecule of claim 6, wherein the linker sequence is a coding sequence of a self-cleaving peptide.

8. The isolated nucleic acid molecule of claim 1, comprising or consisting of a polynucleotide sequence selected from a group consisting of SEQ ID NOs: 21-26.

9. A codon-optimized coding sequence of PGRN, comprising or consisting of a polynucleotide sequence as shown in any one of SEQ ID NOs: 1-8.

10. A codon-optimized coding sequence of STMN2, comprising or consisting of a polynucleotide sequence as shown in any one of SEQ ID NOs: 11-18.

11. An expression cassette, comprising the isolated nucleic acid molecule of claim 1.

12. A recombinant adeno-associated viral (rAAV) vector comprising—the expression cassette of claim 11.

13. The rAAV of claim 12, wherein the rAAV vector is of AAV9 serotype or a ViVec AAV.

14. The rAAV of claim 13, wherein the ViVec AAV has a capsid polypeptide obtained by inserting 7 amino acids at a position between the amino acid position Q588 and the amino acid position A589 of the wild-type AAV9 VP1 capsid protein as shown in SEQ ID NO: 34, and the said ViVec AAV has an increased tropism for one or more tissues or cells of the central nervous system (CNS), and/or is capable of producing higher levels of transgene expression in tissues or cells of the central nervous system, as compared to rAAV having a capsid of the wild-type serotype AAV9.

15. The rAAV of claim 14, wherein the insertion of 7 amino acids is as shown in any one of SEQ ID NOs: 35-94.

16. A viral particle comprising the rAAV vector of claim 12.

17. A pharmaceutical composition, comprising the viral particle of claim 16 and a pharmaceutically acceptable excipient.

18. A method of treating or preventing a neurodegenerative disorder (ND) in a subject in need thereof, comprising administering the pharmaceutical composition of claim 17 to the subject.

19. The method of claim 18, wherein the neurodegenerative disorder is selected from the group consisting of amyotrophic lateral sclerosis (ALS), Frontotemporal Degeneration (FTD), Huntington's disease (HD), Parkinson's disease (PD), multiple system atrophy (MSA), and Alzheimer disease (AD).

20. The method of claim 18, wherein the neurodegenerative disorder is associated with TDP-43 aggregation.
